(19) United States
(12) Patent Application Publication
(10) Pub. No.: US 2024/0034717 A1
(43) Pub. Date: Feb. 1, 2024

(54) NOVEL POTASSIUM CHANNEL INHIBITORS

(71) Applicant: Saniona A/S, Glostrup (DK)

(72) Inventors: David Tristram BROWN, Glostrup (DK); Palle CHRISTOPHERSEN, Glostrup (DK); Thomas Amos JACOBSEN, Glostrup (DK); Janus S. LARSEN, Glostrup (DK); Pernille Hartveit POULSEN, Glostrup (DK); Dorte STRØBAEK, Glostrup (DK)

(21) Appl. No.: 18/246,074

(22) PCT Filed: Sep. 21, 2021
(86) PCT No.: PCT/EP21/75923

§ 371 Date: Mar. 21, 2023

(30) Foreign Application Priority Data
Sep. 22, 2020 (EP) 20197467.2

(51) Int. Cl. C07D 207/09; C07C 25/02

(52) U.S. Cl. CPC C07D 207/09; C07C 25/02

(57) ABSTRACT

The present invention relates to novel compounds, pharmaceutical compositions comprising such compounds and their use for treating, alleviating or preventing diseases or disorders relating to the activity of potassium channels.

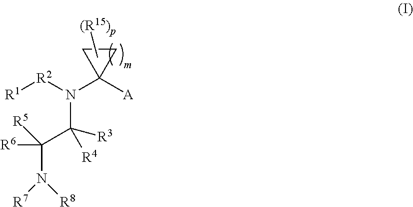

### TECHNICAL FIELD

The present invention relates to novel compounds, pharmaceutical compositions comprising such compounds and their use for treating, alleviating or preventing diseases or disorders relating to the activity of potassium channels.

### BACKGROUND

Ion channels are trans-membrane proteins, which catalyse the transport of inorganic ions across cell membranes. The ion channels participate in very diverse processes among which is the generation and timing of action potentials, synaptic transmission, secretion of hormones, and contraction of muscles.

All mammalian cells express potassium (K⁺) channels in their cell membranes, and the channels play a dominant role in the regulation of the membrane potential. In nerve and muscle cells they influence the form of the action potential, regulate the frequency and firing patterns of action potentials, the release of neurotransmitters as well as the degree of bronchodilation and vasodilation. In non-excitable cells K⁺ channels regulate cellular proliferation and migration as well as the secretion of cytokines.

From a molecular and functional point of view, the K⁺ channels represent the largest and most diverse group of ion channels. It can be divided into four broad families:
voltage-activated K⁺ channels (Kᵥ),
inward rectifier K⁺ channels (KIR),
two-pore K⁺ channels (K2P), and
calcium-activated K⁺ channels (K_{Ca}).

In the K_{Ca} channels, two main groups can be distinguished:
the calmodulin-dependent families, consisting of the small conductance (SK's or K_{Ca}2.x) and intermediate conductance channels (IK or K_{Ca}3.1), and
the intracellular ligand gated families, consisting of the classic Ca²⁺— and voltage-activated big conductance channel (BK, K_{Ca}1.1) as well as channels sensitive to other intracellular ions (K_{Ca}4.x; and K_{Ca}5.1).

K_{Ca}3.1

K_{Ca}3.1 is a Ca²⁺-activated K⁺ channel encoded by the human gene KCNN4. The channel is a tetramer consisting of four identical α-subunits creating the transmembrane K⁺ selective pore at their interfaces, and—at the intracellular side—four calmodulins, which bind incoming Ca²⁺ and open the pore for K⁺ efflux. K_{Ca}3.1 is expressed in many immune cells incl. T- and B-lymphocytes, mast cells, neutrophils, and macrophages, as well as in erythrocytes, fibroblasts, epithelia and endothelia, whereas K_{Ca}3.1 is essentially absent from excitable cells, such as heart, smooth, and striated muscles, and neurons. Furthermore, since K_{Ca}3.1 is essentially absent from excitable cells, pharmacological modulation of this channel is not expected to cause cardiovascular and CNS related adverse effects.

K_{Ca}3.1 in Immune Cells

The role of K_{Ca}3.1 in immune cells is here described for T-cells but is also valid for other immune cells and for fibroblasts. Activated T-cells (including Th0, Th1 and Th2) require sustained high and strictly controlled intracellular Ca²⁺-concentration to orchestrate activation of enzymes and nuclear transcription factors (eg. the Ca²⁺-dependent calcineurine/NFAT system) for control of the immune response. Cytosolic Ca²⁺ is dynamically regulated via intracellular stores, but long-term Ca²⁺-elevation requires influx from the extracellular space. This causes membrane depolarization, which reduces further influx and quickly terminates the process if not counteracted. This is achieved by K_{Ca}3.1 activation and K⁺ efflux keeping the membrane potential negative. Molecular adaptations occur to consolidate the mechanism long-term: The K_{Ca}3.1 channel is phosphorylated by the H-kinase NDPK-B, which increases its maximal activity, and K_{Ca}3.1 expression is upregulated secondary to NFAT activation. Both processes strengthen the hyperpolarizing capacity of Ca²⁺ mediated K_{Ca}3.1 activation.

Efficient maintenance of high-level cytosolic Ca²⁺ homeostasis is beneficial in controlled immune reactions, while it can be severely pathogenic if becoming an uncontrolled autonomous process.

K_{Ca}3.1 in Erythrocytes

Erythrocytes travel between lungs, where O₂ is picked up from alveolar air, and all other tissues, where O₂ is delivered for use in oxidative phosphorylation. The gas exchange occurs in the smallest blood vessels and the erythrocyte needs to be flexible and adapt size to pass the capillary bed.

In this process, K_{Ca}3.1 is activated by the Ca²⁺-influx through Piezo1, which is a Ca²⁺— permeable channel that is turned-on by the mechanical stress to the membrane during passage. K⁺ efflux then drives Cl⁻ and water efflux resulting in a fast and transient shrinkage allowing a smooth passage. Safe on the other side, where the blood vessels widen out again, both channels close and the salt (K⁺, Cl⁻, Ca²⁺) and water gradients are quickly restored by active transport processes, making the erythrocyte ready for the next passage.

Potassium Channel Modulators

Consequently, compounds acting as potassium channel modulating agents may be very useful in the treatment, alleviation and/or prevention of diseases like inflammatory bowel diseases (IBD), xerocytosis erythrocytes and acute respiratory distress syndrome (ARDS).

WO 2014/001363 discloses tetrazole derivatives functioning as potassium channel modulators, which are suitable for use in treating diseases and disorders relating to the activity of potassium channels.

WO 2013/191984 discloses fused thiazine-3-ones, which are suitable for the treatment of diseases related to K_{Ca}3.1.

WO 2014/067861 discloses 3,4-disubstituted oxazolidinone derivatives and their use as inhibitors of calcium activated potassium channel.

Strøbaek et al. (2013) discloses the K(Ca) 3.1 channel inhibitor4-[[3-(Trifluoromethyl)-phenyl]methyl]-2H-1,4-benzothiazin-3(4H)-one (NS6180).

K_{Ca}3.1 is known to play an essential role in diseases such as IBD, hereditary xerocytosis, and ARDS, and thus K_{Ca}3.1 is a promising target for treatment of these diseases. Hence, there is a need for provision of K_{Ca}3.1 modulators.

Many known potassium channel modulating agents have poor solubility in water. Thus, there is a further need for potassium channel modulators, such as K_{Ca}3.1 modulators, which are more soluble in water.

### SUMMARY

In one aspect, the present invention concerns a compound of formula (I):

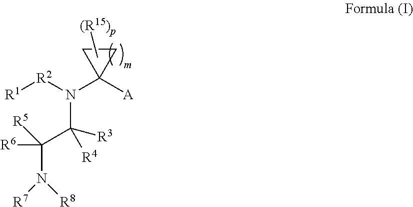

wherein
R¹ is —OC₁₋₈ alkyl; —C₁₋₈ alkyl, optionally substituted with —OH; or H;
R² is a bond; —C(O)—; —S(O)₂—; or —C(H)₂—;
R³ is H; C₁₋₅ alkyl; or a bond;
R⁴ is H; C₁₋₅ alkyl; or a bond;
R⁵ is H; a bond; or C₁₋₈ alkyl, wherein one methylene group optionally is replaced by —O—;
R⁶ is H; a bond; or C₁₋₈ alkyl, wherein one methylene group optionally is replaced by —O—;
R⁷ is H; a bond; —OH; or C₁₋₈ alkyl, wherein one or more methylene group optionally and individually is replaced by —O— and/or substituted with ═O;
R⁸ is H; a bond; —OH; or C₁₋₈ alkyl, wherein one or more methylene group optionally and individually is replaced by —O— and/or substituted with ═O;
R¹⁵ is individually selected from the group consisting of C₁₋₃ alkyl; —OH; —CN; and —F;
anyone of R³, R⁴, R⁵, R⁶, R⁷, and R⁸ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
X is halogen;
m is an integer of 1 to 3; and
p is an integer of 0 to 8;
or a pharmaceutically acceptable salt thereof,
with the proviso that p is not 0 when m is 1, and
with the proviso that the compound is not a compound selected from the group consisting of:
(2R)—N-[1-(4-fluorophenyl)cyclobutyl]-α-methyl-2-pyrrolidinemethanamine;
(2S)—N-[1-(4-fluorophenyl)cyclobutyl]-α-methyl-2-pyrrolidinemethanamine;
N1-[1-(3-bromophenyl)cyclobutyl]-1,2-ethanediamine;
N1-[1-(4-fluorophenyl)cyclobutyl]-1,2-ethanediamine;
N1-[1-(4-chlorophenyl)cyclobutyl]-1,2-ethanediamine;
N-(1-phenylcyclobutyl)-3-azetidinamine;
N1-[1-(4-bromophenyl)cyclobutyl]-1,2-ethanediamine;
(5S)-5-[[[1-(4-fluorophenyl)cyclopentyl]amino]methyl]-2-pyrrolidinone;
N1-[1-(4-fluorophenyl)cyclopentyl]-1,2-ethanediamine; and
N1-[1-(3-methylphenyl)cyclopentyl]-1,2-ethanediamine.

In a further aspect, the present invention relates to a pharmaceutical composition comprising the compound as disclosed herein.

In one aspect, the present invention relates to a compound of formula (I):

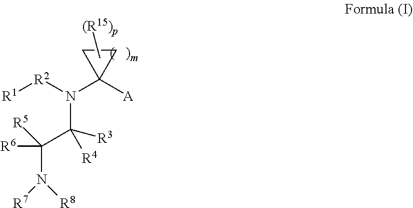

wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R³ is H, a bond, —OH, or C₁₋₈ alkyl, wherein one or more methylene group optionally and individually is replaced by —O— and/or substituted with ═O;
R¹⁵ is individually selected from the group consisting of C₁₋₃ alkyl, —OH, —CN, and —F; anyone of R³, R⁴, R⁵, R⁶, R⁷, and R³ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
X is halogen;
m is an integer of 1 to 4; and
p is an integer of 0 to 10;
or a pharmaceutically acceptable salt thereof,
with the proviso that p is not 0 when m is 1, for use in medicine.

Compounds of the present invention have a high solubility in aqueous medium. Furthermore, compounds of the present invention are active as potassium channel modulators. They are therefore of great interest for the treatment, alleviation and/or prevention of diseases related to potassium channels. Hence, the present invention also relates to the use of a compound as disclosed herein as a medicament. In one aspect, the compounds disclosed herein are used in the treatment of inflammatory bowel disease (IBD). In another aspect, the compounds as disclosed herein are used in the treatment of hereditary xerocytosis. In yet another aspect, the compounds as disclosed herein are used in the treatment of acute respiratory distress syndrome (ARDS).

### DETAILED DESCRIPTION

### Compounds

In one aspect, the present invention relates to a compound of formula (I):

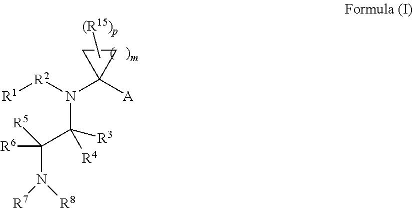

wherein
R¹ is —OC₁₋₃ alkyl, —C₁₋₃ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R⁸ is H, a bond, —OH, or C₁₋₈ alkyl, wherein one or more methylene group optionally and individually is replaced by —O— and/or substituted with ═O;
R¹⁵ is individually selected from the group consisting of C₁₋₃ alkyl, —OH, —CN, and —F; anyone of R³, R⁴, R⁵, R⁶, R⁷, and R⁸ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
X is halogen;
m is an integer of 1 to 4; and
p is an integer of 0 to 10;
or a pharmaceutically acceptable salt thereof.

It is well understood that the term “C₁₋₁₀ alkyl” comprises C₁ alkyl, C₂ alkyl, C₃ alkyls, C₄ alkyls, C₅ alkyls, C₆ alkyls, C₇ alkyls, C₈ alkyls, C₉ alkyls, and Cao alkyl. Said alkyl may be linear, branched and/or cyclic. Thus, said alkyl may be partly cyclic. For example, “C₁-C₆-alkyl” designates an alkyl group containing from 1 to 6 carbon atoms that can be linear or branched such as methyl, ethyl, prop-1-yl, prop-2-yl, iso-propyl, tert-butyl, but-1-yl, but-2-yl, pent-1-yl, pent-2-yl, pent-3-yl, 2-methylbut-1-yl, 3-methylbut-1-yl), hex-1-yl or 2,3-dimethylbut-1-yl.

For example, “C₃-C₇-cycloalkyl” designates a saturated monocyclic carbocyclic ring containing from 3 to 7 carbon atoms such as cyclopropyl, cyclobutyl, cyclopentyl, cyclohexyl or cycloheptyl.

For example, “C₁-C₆-alkoxy” designates a —O—C₁-C₆-alkyl group such as methoxy, ethoxy, 1-propoxy, 2-propoxy, 1-butoxy, 2-butoxy, 2-methyl-2-propoxy, 1-pentoxy, 3-methyl-1-butoxy, 2-pentoxy, 2-methyl-2-butoxy, 1-hexoxy or 3-hexoxy.

In one aspect, the present invention concerns a compound of formula (I):

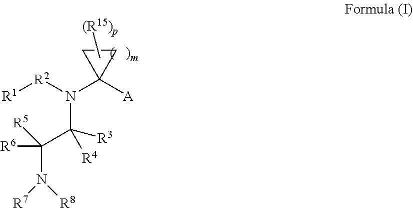

wherein
R¹ is —OC₁₋₈ alkyl; —C₁₋₈ alkyl, optionally substituted with —OH; or H;
R² is a bond; —C(O)—; —S(O)₂—; or —C(H)₂—;
R³ is H; C₁₋₅ alkyl; or a bond;
R⁴ is H; C₁₋₅ alkyl; or a bond;
R⁵ is H; a bond; or C₁₋₈ alkyl, wherein one methylene group optionally is replaced by —O—;
R⁶ is H; a bond; or C₁₋₈ alkyl, wherein one methylene group optionally is replaced by —O—;
R⁷ is H; a bond; —OH; or C₁₋₈ alkyl, wherein one or more methylene group optionally and individually is replaced by —O— and/or substituted with ═O;
R³ is H; a bond; —OH; or C₁₋₈ alkyl, wherein one or more methylene group optionally and individually is replaced by —O— and/or substituted with ═O;
R¹⁵ is individually selected from the group consisting of C₁₋₃ alkyl; —OH; —CN; and —F;
anyone of R³, R⁴, R⁵, R⁶, R⁷, and R³ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
X is halogen;
m is an integer of 1 to 3; and
p is an integer of 0 to 8;
or a pharmaceutically acceptable salt thereof,
with the proviso that p is not 0 when m is 1, and
with the proviso that the compound is not a compound selected from the group consisting of:
(2R)—N-[1-(4-fluorophenyl)cyclobutyl]-α-methyl-2-pyrrolidinemethanamine;
(2S)—N-[1-(4-fluorophenyl)cyclobutyl]-α-methyl-2-pyrrolidinemethanamine;
N1-[1-(3-bromophenyl)cyclobutyl]-1,2-ethanediamine;
N1-[1-(4-fluorophenyl)cyclobutyl]-1,2-ethanediamine;
N1-[1-(4-chlorophenyl)cyclobutyl]-1,2-ethanediamine;
N-(1-phenylcyclobutyl)-3-azetidinamine;
N1-[1-(4-bromophenyl)cyclobutyl]-1,2-ethanediamine;
(5S)-5-[[[1-(4-fluorophenyl)cyclopentyl]amino]methyl]-2-pyrrolidinone;
N1-[1-(4-fluorophenyl)cyclopentyl]-1,2-ethanediamine; and
N1-[1-(3-methylphenyl)cyclopentyl]-1,2-ethanediamine.

In one embodiment, when m is 1 then p is not 0. In one embodiment, m is 1 and p is an integer of 1 to 4.

In one embodiment, m is 2. In one embodiment, the compound is of formula (II):

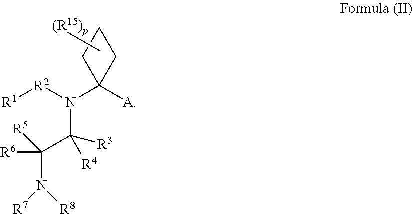

In one embodiment, m is 3. In one embodiment, the compound is of formula (III):

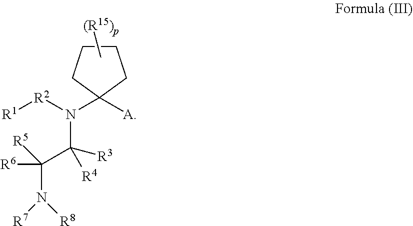

In one embodiment, the compound is of formula (IV):

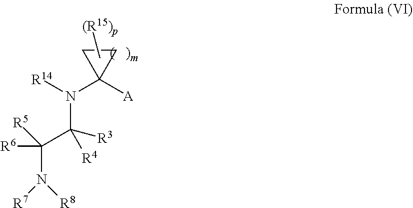

wherein
R¹⁴ is selected from the group consisting of —C(O)—C₁₋₈ alkyl; —C(O)—O—C₁₋₈ alkyl; —C₂₋₈ alkyl; —H and —S(O)₂—C₁₋₈ alkyl;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R⁸ is H, a bond, —OH, or C₁₋₈ alkyl, wherein one or more methylene group optionally and individually is replaced by —O— and/or substituted with ═O;
R¹⁵ is individually selected from the group consisting of C₁₋₃ alkyl, —OH, —CN, and —F; anyone of R³, R⁴, R⁵, R⁶, R⁷, and R⁸ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅; and
X is halogen;
m is an integer of 1 to 4; and
p is an integer of 0 to 10;
or a pharmaceutically acceptable salt thereof.

In one embodiment, the compound is of formula (XV):

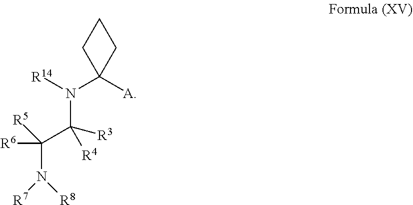

In one embodiment, A is a moiety of formula (XII):

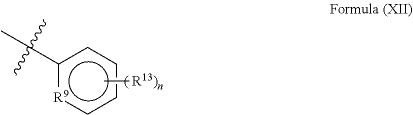

wherein
R⁹ is —C(H)—, —N—, or —C(R¹³)—;
R¹³ is individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
n is an integer of 0 to 4; and
X is halogen.

In one embodiment, the compound is of formula (V):

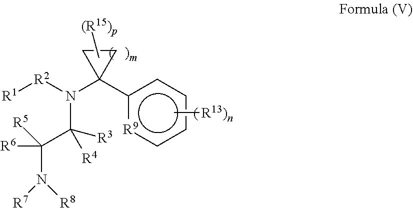

wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R⁸ is H, a bond, —OH, or C₁₋₈ alkyl, wherein one or more methylene group optionally and individually is replaced by —O— and/or substituted with ═O;
R⁹ is —C(H)—, —N—, or —C(R¹³)—;
R¹³ is individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
R¹⁵ is individually selected from the group consisting of C₁₋₂ alkyl, —OH, —CN, and —F; anyone of R³, R⁴, R⁵, R⁶, R⁷, and R⁸ optionally is linked together to form a ring;
X is halogen;
n is an integer of 0 to 4;
m is an integer of 1 to 4; and
p is an integer of 0 to 10;
or a pharmaceutically acceptable salt thereof.

In one embodiment, the compound is of formula (V):

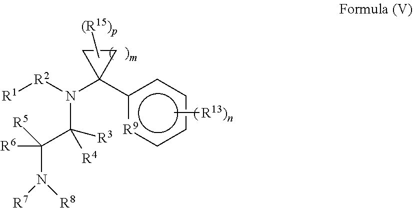

wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R⁸ is H, a bond, —OH, or C₁₋₈ alkyl, wherein one or more methylene group optionally and individually is replaced by —O— and/or substituted with ═O;
R⁹ is —C(H)—, —N—, or —C(R¹³)—;
R¹³ is individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
R¹⁵ is individually selected from the group consisting of C₁₋₂ alkyl, —OH, —CN, and —F; anyone of R³, R⁴, R⁵, R⁶, R⁷, and R⁸ optionally is linked together to form a ring;
X is halogen;
n is an integer of 0 to 4;
m is an integer of 1 to 3; and
p is an integer of 0 to 8;
or a pharmaceutically acceptable salt thereof.

In one embodiment, A is a moiety of formula (XIII):

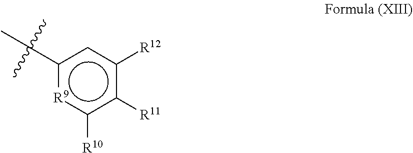

wherein
R⁹ is —C(H)—, —N—, or —C(R¹³)—;
R¹⁰, R¹¹, R¹², and R¹³ are individually selected from the group consisting of H, halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅; and
X is halogen.

In one embodiment, the compound is of formula (VI):

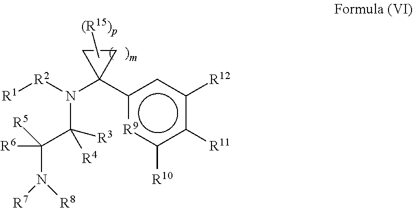

wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R⁸ is H, a bond, —OH, or C₁₋₈ alkyl, wherein one or more methylene group optionally and individually is replaced by —O— and/or substituted with ═O;
anyone of R³, R⁴, R⁵, R⁶, R⁷, and R⁸ optionally is linked together to form a ring;
R⁹ is —C(H)—, —N—, or —C(R¹³)—;
R¹⁰, R¹¹, R¹², and R¹³ are individually selected from the group consisting of H, halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
R¹⁵ is individually selected from the group consisting of C₁₋₃ alkyl, —OH, —CN, and —F;
X is halogen;
m is an integer of 1 to 4; and
p is an integer of 0 to 10;
or a pharmaceutically acceptable salt thereof.

In one embodiment, the compound is of formula (VI):

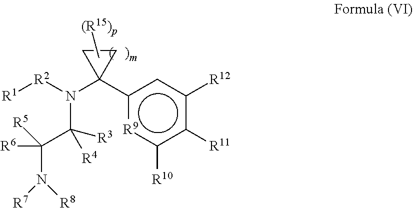

wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R⁸ is H, a bond, —OH, or C₁₋₈ alkyl, wherein one or more methylene group optionally and individually is replaced by —O— and/or substituted with ═O;
anyone of R³, R⁴, R⁵, R⁶, R⁷, and R⁸ optionally is linked together to form a ring;
R⁹ is —C(H)—, —N—, or —C(R¹³)—;
R¹⁰, R¹¹, R¹², and R¹³ are individually selected from the group consisting of H, halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
R¹⁵ is individually selected from the group consisting of C₁₋₃ alkyl, —OH, —CN, and —F;
X is halogen;
m is an integer of 1 to 3; and
p is an integer of 0 to 8;
or a pharmaceutically acceptable salt thereof.

In one embodiment, the compound is of formula (VI):

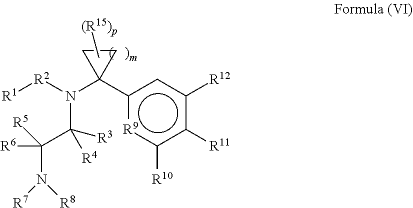

wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R⁸ is H, a bond, —OH, or C₁₋₈ alkyl, wherein one or more methylene group optionally and individually is replaced by —O— and/or substituted with ═O;
anyone of R³, R⁴, R⁵, R⁶, R⁷, and R⁸ optionally is linked together to form a ring;
R⁹ is —C(H)— or —N—;
R¹⁰ is H or halogen;
R¹¹ is H or halogen;
R¹² is —CX₃, —OCX₃, H, halogen, —C₁₋₈ alkyl, or —C₃₋₇ cycloalkyl;
R¹⁵ is individually selected from the group consisting of C₁₋₃ alkyl, —OH, —CN, and —F
X is halogen;
m is an integer of 1 to 4; and
p is an integer of 0 to 10;
or a pharmaceutically acceptable salt thereof.

In one embodiment, the compound is of formula (VI):

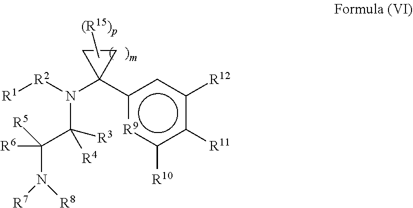

wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R⁸ is H, a bond, —OH, or C₁₋₈ alkyl, wherein one or more methylene group optionally and individually is replaced by —O— and/or substituted with ═O;
anyone of R³, R⁴, R⁵, R⁶, R⁷, and R⁸ optionally is linked together to form a ring;
R⁹ is —C(H)— or —N—;
R¹⁰ is H or halogen;
R¹¹ is H or halogen;
R¹² is —CX₃, —OCX₃, H, halogen, —C₁₋₈ alkyl, or —C₃₋₇ cycloalkyl;
R¹⁵ is individually selected from the group consisting of C₁₋₃ alkyl, —OH, —CN, and —F
X is halogen;
m is an integer of 1 to 3; and
p is an integer of 0 to 8;
or a pharmaceutically acceptable salt thereof.

In one embodiment, p is 0.

In one embodiment, R¹ is —OC₁₋₈ alkyl, such as —OC₁₋₇ alkyl, such as —OC₁₋₆ alkyl, such as —OC₁₋₅ alkyl, such as —OC₁₋₄ alkyl, such as —OC₁₋₃ alkyl, such as —OC₁₋₂ alkyl, such as —OC alkyl. In one embodiment, R¹ is —C₁₋₈ alkyl, such as —C₁₋₇ alkyl, such as —C₁₋₆ alkyl, such as —C₁₋₅ alkyl, such as —C₁₋₄ alkyl, such as —C₁₋₃ alkyl, such as —C₁₋₂ alkyl, such as —C₁ alkyl. In one embodiment, R¹ is —C₁₋₈ alkyl substituted with —OH. In one embodiment, R¹ is —H.

In one embodiment, R² is a bond. In one embodiment, R² is-C(O)—. In one embodiment, R² is —C(H)₂—. In one embodiment, R² is —S(O)₂—. In one embodiment, R² is —C(O)— and R¹ is —OC₁₋₄ alkyl. In one embodiment, R² is —C(O)— and R¹ is —OC₁₋₃ alkyl.

In one embodiment, R² is a bond and R¹ is C₃₋₄ alkyl. In one embodiment, R¹ is —OC₁₋₈ alkyl, or —C₁₋₈ alkyl, optionally substituted with —OH, and R² is a bond, —C(O)—, —S(O)₂—, or —C(H)₂—. In one embodiment, —R¹-R² is not H. In one embodiment, —R¹-R² is not —CH₃.

In one embodiment, —R²-R¹ is —R¹⁴, and R¹⁴ is selected from the group consisting of —C(O)—C₁₋₃ alkyl; —C(O)—O—C₁₋₃ alkyl; —C₂₋₈ alkyl; —H and —S(O)₂—C₁₋₈ alkyl. In one embodiment, —R²-R¹ is —R¹⁴, and R¹⁴ is selected from the group consisting of —C(O)—C₁₋₈ alkyl; —C(O)—O—C₁₋₃ alkyl; —C₂₋₈ alkyl; and —S(O)₂—C₁₋₈ alkyl. In one embodiment, R¹⁴ is —C(O)—C₁₋₃ alkyl, such as R¹⁴ is —C(O)—C₁₋₃ alkyl, such as R¹⁴ is —C(O)—C₃ alkyl, such as —C(O)-cyclopropyl. In one embodiment, R¹⁴ is —C(O)—O—C₁₋₃ alkyl, such as, R¹⁴ is —C(O)—OC₁₋₃ alkyl, such as R¹⁴ is selected from the group consisting of —C(O)—OCH₃, —C(O)—OCH₂CH₃, —OCH₂(CH₃)₂ and —O-cyclopropyl. In one embodiment, R¹⁴ is —C₂₋₈ alkyl, such as C₃₋₄ alkyl. In one embodiment, R¹⁴ is —C(H)₂—C₃₋₇ cycloalkyl, such as —C(H)₂-cyclopropyl or —C(H)₂-cyclobutyl. In one embodiment, R¹⁴ is —C₃₋₇ cycloalkyl, such as—cyclopropyl or—cyclobutyl. In one embodiment, R¹⁴ is —C₂₋₈ alkyl, such as C₃₋₄ alkyl, substituted with one or more —OH, such as R¹⁴ is isopropyl substituted with —OH. In one embodiment, R¹⁴ is —H. In one embodiment, R¹⁴ is —S(O)₂—C₁₋₈ alkyl, such as R¹⁴ is —S(O)₂—CH₃. In one embodiment, R¹⁴ is not —CH₃. In one embodiment, R¹⁴ is not H.

In one embodiment, R³ is H. In another embodiment, R³ is a bond. In one embodiment, R³ is C₁₋₅ alkyl, such as C₁₋₄ alkyl, such as C₁₋₃ alkyl, such as C₁₋₂ alkyl, such as C₁₋₈ alkyl. Said alkyl may be linear, branched, cyclic or partly cyclic.

In one embodiment, R⁴ is H. In another embodiment, R⁴ is a bond. In one embodiment, R⁴ is C₁₋₅ alkyl, such as C₁₋₄ alkyl, such as C₁₋₃ alkyl, such as C₁₋₂ alkyl, such as C₁₋₈ alkyl. Said alkyl may be linear, branched, cyclic or partly cyclic.

In one embodiment, R³ and R⁴ are H. In another embodiment, only one of R³ and R⁴ are H, whereas the other is a bond or C₁₋₅ alkyl.

In one embodiment, R⁵ is H. In one embodiment, R⁵ is a bond. In one embodiment, R⁵ is C₁₋₃ alkyl, such as C₁₋₇ alkyl, such as C₁₋₆ alkyl, such as C₁₋₅ alkyl, such as C₁₋₄ alkyl, such as C₁₋₃ alkyl, such as C₁₋₂ alkyl, such as C₁ alkyl. In one embodiment, one of the methylene groups in said alkyl is replaced by —O—, thus forming an ether moiety. In one embodiment, R⁵ is C₁₋₄ alkyl.

In one embodiment, R⁶ is H. In one embodiment, R⁶ is a bond. In one embodiment, R⁶ is C₁₋₈ alkyl, such as C₁₋₇ alkyl, such as C₁₋₆ alkyl, such as C₁₋₅ alkyl, such as C₁₋₄ alkyl, such as C₁₋₃ alkyl, such as C₁₋₂ alkyl, such as C₁ alkyl. In one embodiment, one of the methylene groups in said alkyl is replaced by —O—, thus forming an ether moiety. In one embodiment, R⁶ is C₁₋₄ alkyl.

In one embodiment, R⁵ and R⁶ are H. In one embodiment, R⁵ and R⁶ are —CH₃. In one embodiment, R⁵ and R⁶ are linked together to form a ring. Said ring may be a three-membered ring, a four-membered ring, a five-membered ring, a six-membered ring, or a seven-membered ring. In one embodiment, said ring is a three-membered ring. In another embodiment, only one of R⁵ and R⁶ are H, whereas the other is a bond or C₁₋₈ alkyl. In one embodiment, R⁵ and R⁶ are linked together to form a ring as in formula (XI):

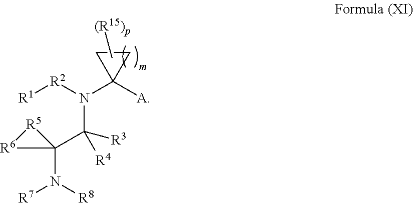

In one embodiment, R⁷ is H. In one embodiment, R⁷ is a bond. In one embodiment, R⁷ is C₁₋₈ alkyl, such as C₁₋₇ alkyl, such as C₁₋₆ alkyl, such as C₁₋₅ alkyl, such as C₁₋₄ alkyl, such as C₁₋₃ alkyl, such as C₁₋₂ alkyl, such as C₁ alkyl. In one embodiment, one or more methylene group of said alkyl is replaced by —O—. In one embodiment, R⁷ is —C(O)—O—CH₃ or —C(O)—CH₃.

In one embodiment, R⁸ is H. In one embodiment, R⁸ is a bond. In one embodiment, R⁸ is C₁₋₈ alkyl, such as C₁₋₇ alkyl, such as C₁₋₆ alkyl, such as C₁₋₅ alkyl, such as C₁₋₄ alkyl, such as C₁₋₃ alkyl, such as C₁₋₂ alkyl, such as C₁ alkyl. In one embodiment, one or more methylene group of said alkyl is replaced by —O—.

In one embodiment, R⁷ is selected from the group consisting of H, a bond, —OH, or C₁₋₈ alkyl, wherein one or more methylene group optionally and individually is replaced by —O—; and R⁸ is selected from the group consisting of H, a bond, —OH, or C₁₋₈ alkyl, wherein one or more methylene group optionally and individually is replaced by —O—.

In one embodiment, R⁵ or R⁶ is linked to R⁷ or R⁸ to form a ring, such as R⁵ is linked to R⁷. In one embodiment, when R⁵ is linked to R⁷ then R⁶ and R⁸ are H. In one embodiment, when R⁵ is linked to R⁷ then R⁶ is H and R⁸ is methyl. In one embodiment, the ring formed by R⁵ or R⁶ linked to R⁷ or R⁸, such as R⁵ is linked to R⁷, is a four-membered ring, a five-membered ring, a six-membered ring, a three-membered ring or a seven-membered ring. In one embodiment, the ring formed when R⁵ or R⁶ is linked to R⁷ or R⁸, such as when R⁵ is linked to R⁷, is an azetidine. In one embodiment, the ring formed when R⁵ or R⁶ is linked to R⁷ or R⁸, such as when R⁵ is linked to R⁷, is a pyrrolidine. In one embodiment, the ring formed when R⁵ or R⁶ is linked to R⁷ or R⁸, such as when R⁵ is linked to R⁷, is a morpholine. In one embodiment, the ring formed when R⁵ or R⁶ is linked to R⁷ or R⁸, such as when R⁵ is linked to R⁷, is a piperidine. In one embodiment, R⁵ is linked to R⁷ as in formula (VII):

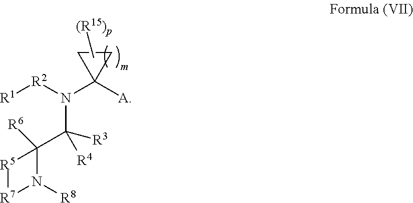

In one embodiment, when the compound is of formula (VII), then R³ and R⁴ are H. In one embodiment, when the compound is of formula (VII) and R³ is H, then R⁴ is not —CH₃. In one embodiment, when the compound is of formula (VII) and R⁸ is H, then R⁷ is selected from the group consisting of H, a bond, —OH, or C₁₋₈ alkyl, wherein one or more methylene group optionally and individually is replaced by —O—.

In one embodiment, the compound is of formula (VIII):

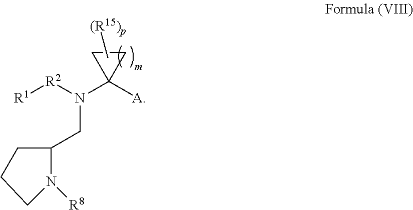

In one embodiment, the compound is of formula (XIV):

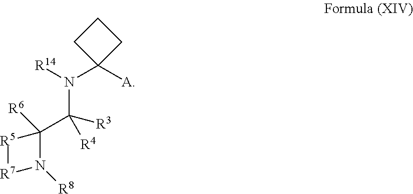

In one embodiment, R³ or R⁴ is linked to R⁷ or R⁸ to form a ring, such as R³ is linked to R⁷. In one embodiment, when R³ is linked to R⁷ then R⁴ and R⁸ are H. In one embodiment, the ring formed by R³ or R⁴ linked to R⁷ or R⁸, such as R³ linked to R⁷, is a four-membered ring, a five-membered ring, a six-membered ring, a three-membered ring or a seven-membered ring. In one embodiment, the ring formed when R³ or R⁴ is linked to R⁷ or R⁸, such as when R⁵ is linked to R⁷, is a four-membered ring. In one embodiment, the ring formed when R³ or R⁴ is linked to R⁷ or R⁸, such as when R³ is linked to R⁷, is an azetidine. In one embodiment, R³ is linked to R⁷ as in formula (X):

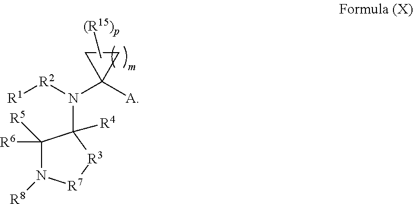

In one embodiment, when the compound is of formula (X) and —R³-R⁷ is —CH₂—, then —R—R² is not H.

In one embodiment, when the compound is of formula (X) and —R³-R⁷ is —CH₂—, then A is substituted with at least one substituent R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅.

In one embodiment, R³ or R⁴ is linked to R⁵ or R⁶ to form a ring, such as R³ is linked to R⁵. In one embodiment, when R³ is linked to R⁵ then R⁴ and R⁶ are H. In one embodiment, the ring formed by R³ or R⁴ linked to R⁵ or R⁶, such as R³ linked to R⁵, is a five-membered ring, a four-membered ring, a six-membered ring, a three-membered ring or a seven-membered ring. In one embodiment, the ring formed when R³ or R⁴ is linked to R⁵ or R⁶, such as when R³ is linked to R⁵, is a five-membered ring. In one embodiment, the ring formed when R³ or R⁴ is linked to R⁵ or R⁶, such as when R³ is linked to R⁵, is a four-membered ring. In one embodiment, the ring formed when R³ or R⁴ is linked to R⁵ or R⁶, such as when R³ is linked to R⁵, is a cyclopentyl. In one embodiment, R³ is linked to R⁵ as in formula (IX):

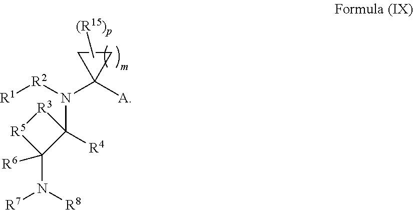

In one embodiment, R³ and R⁴ are —H, R⁵ and R⁶ are methyl, and R⁷ and R⁸ are —H.

In one embodiment, R⁹ is —C(H)—. In one embodiment, R⁹ is —C(F)—.

In one embodiment, R¹⁰, R¹¹ and R¹² are individually selected from the group consisting of H, halogen, —CX₃, —OCX₃, —C₁₋₈ alkyl, and —C₃₋₇ cycloalkyl. In one embodiment, R¹⁰, R¹¹ and R¹² are individually selected from the group consisting of H, halogen, —CX₃, and —OCX₃.

In one embodiment, R¹⁰ is H. In one embodiment, R¹⁰ is F. In one embodiment, R¹⁰ is Cl.

In one embodiment, R¹¹ is F. In one embodiment, R¹¹ is H.

In one embodiment, R¹² is —CX₃, such as —CF₃. In one embodiment, R¹² is —OCF₃. In one embodiment, R¹² is F, Cl or Br. In one embodiment, R¹² is —C₁₋₈ alkyl, such as —C₁ alkyl, such as —C₂ alkyl, such as —C₃ alkyl. In one embodiment, R¹² is —C₃₋₇ cycloalkyl, such as —C₃ cycloalkyl, such as —C₃ cycloalkyl, such as —C₅ cycloalkyl.

In one embodiment, R⁹ is —C(H)— or —N—; R¹⁰ is H or halogen; R¹¹ is H or halogen; R¹² is —CX₃, —OCX₃, H, halogen, —C₁₋₄ alkyl, or —C₃₋₅ cycloalkyl; and X is halogen. In one embodiment, R¹¹ is F and R¹² is —CF₃. In one embodiment, R⁹ is —C(H)—, R¹⁰ is H, R¹¹ is F and R¹² is —CF₃ or —OCF₃. In one embodiment, R⁹ is —C(H)—, R¹⁰ is H, R¹¹ is F and R¹² is —CF₃. In one embodiment, R⁹ is —C(H)—, R¹⁰ is H, R¹¹ is H and R¹² is —CF₃. In one embodiment, R⁹ is —C(H)—, R¹⁰ is F, R¹¹ is H and R¹² is —CF₃. In one embodiment, R⁹ is —C(F)—, R¹⁰ is H, R¹¹ is H and R¹² is —CF₃. In one embodiment, R⁹ is —C(H)—, R¹⁰ is H, R¹¹ is F and R¹² is —OCF₃. In one embodiment, R⁹ is —C(H)—, R¹⁰ is H, R¹¹ is H and R¹² is —OCF₃. In one embodiment, R⁹ is —C(H)—, R¹⁰ is H, R¹¹ is H and R¹² is halogen. In one embodiment, R⁹ is —C(F)—, R¹⁰ is H, R¹¹ is H and R¹² is halogen. In one embodiment, R⁹ is —C(H)—, R¹⁰ is H, R¹¹ is F and R¹² is —C₃ cycloalkyl.

In one embodiment, the compound is the (S)-enantiomer. In another embodiment, the compound is the (R)-enantiomer.

In one embodiment, the moiety A substituted with at least two substituents R¹³. In one embodiment, no more than two of R¹⁰, R¹¹ and R¹² are H. In one embodiment, the moiety A substituted with at least three substituents R¹³. In one embodiment, no more than one of R¹⁰, R¹¹ and R¹² are H.

In one embodiment, when R¹¹ and R¹² are H, then R¹⁰ is halogen.

In one embodiment, no more than five of R³, R⁴, R⁵, R⁶, R⁷ and R⁸ are H.

In one embodiment, when R³, R⁴, R⁵, R⁶, R⁷ and R⁸ are H, then no more than two of R¹⁰, R¹¹ and R¹² are H.

In one embodiment, when R³, R⁴, R⁵, R⁶, R⁷ and R⁸ are H, then A is substituted with at least two substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₃ alkyl, —OC₁₋₈ alkyl, —C₃₋₇ cycloalkyl, —OC₃₋₇ cycloalkyl, —CN, NO₂, —SO₂CH₃, and —SF₅.

In one embodiment, when R³, R⁴, R⁵, R⁶, R⁷ and R⁸ are H, then —R¹-R² is not H. In one embodiment, when R³, R⁴, R⁵, R⁶, R⁷ and R⁸ are H, then —R¹⁴ is not H.

In one embodiment, the present invention relates to compound of formula (I):

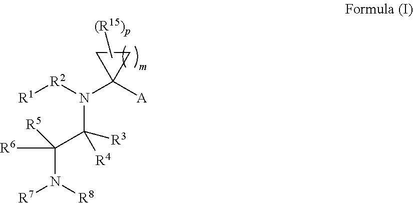

wherein
R¹ is —OC₁₋₈ alkyl; —C₁₋₃ alkyl, optionally substituted with —OH; or H;
R² is a bond; —C(O)—; —S(O)₂—; or —C(H)₂—;
R³ is H; C₁₋₅ alkyl; or a bond;
R⁴ is H; C₁₋₅ alkyl; or a bond;
R⁵ is H; a bond; or C₁₋₈ alkyl, wherein one methylene group optionally is replaced by —O—;
R⁶ is H; a bond; or C₁₋₈ alkyl, wherein one methylene group optionally is replaced by —O—;
R⁷ is H; a bond; —OH; or C₁₋₈ alkyl, wherein one or more methylene group optionally and individually is replaced by —O— and/or substituted with ═O;
R⁸ is H;
anyone of R³, R⁴, R⁵, R⁶, and R⁷ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₃ alkyl, —C₃₋₇ cycloalkyl, —OC₃₋₇ cycloalkyl, —CN, NO₂, —SO₂CH₃, and —SF₅;
X is halogen;
m is an integer of 1 to 4; and
p is an integer of 0 to 10;
or a pharmaceutically acceptable salt thereof,
with the proviso that p is not 0 when m is 1, and
with the proviso that the compound is not a compound selected from the group consisting of:
(2R)—N-[1-(4-fluorophenyl)cyclobutyl]-α-methyl-2-pyrrolidinemethanamine;
(2S)—N-[1-(4-fluorophenyl)cyclobutyl]-α-methyl-2-pyrrolidinemethanamine;
N1-[1-(3-bromophenyl)cyclobutyl]-1,2-ethanediamine;
N1-[1-(4-fluorophenyl)cyclobutyl]-1,2-ethanediamine;
N1-[1-(4-chlorophenyl)cyclobutyl]-1,2-ethanediamine;
N-(1-phenylcyclobutyl)-3-azetidinamine;
N1-[1-(4-bromophenyl)cyclobutyl]-1,2-ethanediamine;
(5S)-5-[[[1-(4-fluorophenyl)cyclopentyl]amino]methyl]-2-pyrrolidinone;
N1-[1-(4-fluorophenyl)cyclopentyl]-1,2-ethanediamine;
N1-[1-(3-methylphenyl)cyclopentyl]-1,2-ethanediamine;
(5S)-5-[[[1-(4-chlorophenyl)cyclohexyl]amino]methyl]-2-pyrrolidinone;
(5S)-5-[[[1-(4-bromophenyl)cyclohexyl]amino]methyl]-2-pyrrolidinone;
(5S)-5-[[[1-(2-bromophenyl)cyclohexyl]amino]methyl]-2-pyrrolidinone;
(5S)-5-[[[1-(3-bromophenyl)cyclohexyl]amino]methyl]-2-pyrrolidinone; and
N1-[1-(4-chlorophenyl)cyclohexyl]-1,2-ethanediamine.

In one embodiment, the current invention relates to a compound of formula (I):

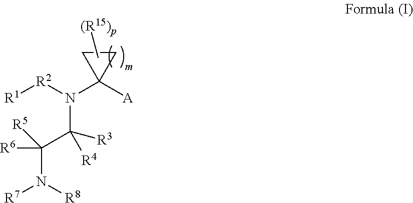

wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R³ is H, a bond, —OH, or C₁₋₈ alkyl, wherein one or more methylene group optionally and individually is replaced by —O— and/or substituted with ═O;
R¹⁵ is individually selected from the group consisting of C₁₋₃ alkyl, —OH, —CN, and —F; anyone of R³, R⁴, R⁵, R⁶, R⁷, and R³ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
X is halogen;
m is an integer of 1 to 4; and
p is an integer of 0 to 10;
or a pharmaceutically acceptable salt thereof,
with the proviso that p is not 0 when m is 1, and with the proviso that the compound is not a compound selected from the group consisting of:
(2R)—N-[1-(4-fluorophenyl)cyclobutyl]-α-methyl-2-pyrrolidinemethanamine;
(2S)—N-[1-(4-fluorophenyl)cyclobutyl]-α-methyl-2-pyrrolidinemethanamine;
N1-[1-(3-bromophenyl)cyclobutyl]-1,2-ethanediamine;
N1-[1-(4-fluorophenyl)cyclobutyl]-1,2-ethanediamine;
N1-[1-(4-chlorophenyl)cyclobutyl]-1,2-ethanediamine;
N-(1-phenylcyclobutyl)-3-azetidinamine;
N1-[1-(4-bromophenyl)cyclobutyl]-1,2-ethanediamine;
(5S)-5-[[[1-(4-fluorophenyl)cyclopentyl]amino]methyl]-2-pyrrolidinone;
N1-[1-(4-fluorophenyl)cyclopentyl]-1,2-ethanediamine;
N1-[1-(3-methylphenyl)cyclopentyl]-1,2-ethanediamine;
(5S)-5-[[[1-(4-chlorophenyl)cyclohexyl]amino]methyl]-2-pyrrolidinone;
(5S)-5-[[[1-(4-bromophenyl)cyclohexyl]amino]methyl]-2-pyrrolidinone;
(5S)-5-[[[1-(2-bromophenyl)cyclohexyl]amino]methyl]-2-pyrrolidinone;
(5S)-5-[[[1-(3-bromophenyl)cyclohexyl]amino]methyl]-2-pyrrolidinone;
N1-[1-(4-chlorophenyl)cyclohexyl]-1,2-ethanediamine;
N1,N1-diethyl-N2-methyl-N2-(1-phenylcyclohexyl)ethane-1,2-diamine;
N1,N1-diethyl-N2-(1-phenylcyclohexyl)ethane-1,2-diamine;
N-(1-phenylcyclohexyl)-4-morpholineethanamine;
N2-(3,5-dimethyl-1-phenylcyclohexyl)-N1,N1-diethyl-1,2-ethanediamine;
N1,N1-diethyl-N2-(3,3,5-trimethyl-1-phenylcyclohexyl)ethane-1,2-diamine;
N1-(3,5-dimethyl-1-phenylcyclohexyl)-N2,N2-dimethylethane-1,2-diamine;
N-(3,5-dimethyl-1-phenylcyclohexyl)-1-piperidineethanamine;
N-(3,3,5-trimethyl-1-phenylcyclohexyl)-1-piperidineethanamine; and
N-(3,5-dimethyl-1-phenylcyclohexyl)-N-methyl-1-piperidineethanamine.

In one embodiment, the compound is of formula (IV):

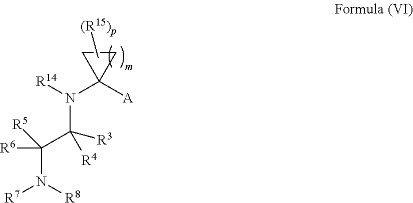

wherein
R¹⁴ is selected from the group consisting of —C(O)—C₁₋₈ alkyl; —C(O)—O—C₁₋₈ alkyl; —C₂₋₈ alkyl; —H and —S(O)₂—C₁₋₈ alkyl;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R³ is H, a bond, —OH, or C₁₋₈ alkyl, wherein one or more methylene group optionally and individually is replaced by —O— and/or substituted with ═O;
R¹⁵ is individually selected from the group consisting of C₁₋₃ alkyl, —OH, —CN, and —F; anyone of R³, R⁴, R⁵, R⁶, R⁷, and R³ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅; and
X is halogen;
m is an integer of 1 to 3; and
p is an integer of 0 to 8;
or a pharmaceutically acceptable salt thereof;
with the proviso that p is not 0 when m is 1, and
with the proviso that the compound is not a compound selected from the group consisting of:
(2R)—N-[1-(4-fluorophenyl)cyclobutyl]-α-methyl-2-pyrrolidinemethanamine;
(2S)—N-[1-(4-fluorophenyl)cyclobutyl]-α-methyl-2-pyrrolidinemethanamine;
N1-[1-(3-bromophenyl)cyclobutyl]-1,2-ethanediamine;
N1-[1-(4-fluorophenyl)cyclobutyl]-1,2-ethanediamine;
N1-[1-(4-chlorophenyl)cyclobutyl]-1,2-ethanediamine;
N-(1-phenylcyclobutyl)-3-azetidinamine;
N1-[1-(4-bromophenyl)cyclobutyl]-1,2-ethanediamine;
(5S)-5-[[[1-(4-fluorophenyl)cyclopentyl]amino]methyl]-2-pyrrolidinone;
N1-[1-(4-fluorophenyl)cyclopentyl]-1,2-ethanediamine; and
N1-[1-(3-methylphenyl)cyclopentyl]-1,2-ethanediamine.

In one embodiment, the compound is not a compound selected from the group consisting of:
(2R)—N-[1-(4-fluorophenyl)cyclobutyl]-α-methyl-2-pyrrolidinemethanamine (CAS number: 2398411-64-6);
(2S)—N-[1-(4-fluorophenyl)cyclobutyl]-α-methyl-2-pyrrolidinemethanamine (CAS number: 2398323-84-5);
N1-[1-(3-bromophenyl)cyclobutyl]-1,2-ethanediamine (CAS number: 1878927-82-2);
N1-[1-(4-fluorophenyl)cyclobutyl]-1,2-ethanediamine (CAS number: 1874820-12-8);
N1-[1-(4-chlorophenyl)cyclobutyl]-1,2-ethanediamine (CAS number: 1873987-18-8);
N-(1-phenylcyclobutyl)-3-azetidinamine (CAS number: 1871338-59-8);
N1-[1-(4-bromophenyl)cyclobutyl]-1,2-ethanediamine (CAS number: 1859512-34-7);
(5S)-5-[[[1-(4-fluorophenyl)cyclopentyl]amino]methyl]-2-pyrrolidinone (CAS number: 2397832-15-2);
N1-[1-(4-fluorophenyl)cyclopentyl]-1,2-ethanediamine (CAS number: 2039942-45-3);
N1-[1-(3-methylphenyl)cyclopentyl]-1,2-ethanediamine (CAS number: 1941061-22-8);
(5S)-5-[[[1-(4-chlorophenyl)cyclohexyl]amino]methyl]-2-pyrrolidinone (CAS number: 2397438-77-4);
(5S)-5-[[[1-(4-bromophenyl)cyclohexyl]amino]methyl]-2-pyrrolidinone (CAS number: 2397389-52-3);
(5S)-5-[[[1-(2-bromophenyl)cyclohexyl]amino]methyl]-2-pyrrolidinone (CAS number: 2397370-60-2);
(5S)-5-[[[1-(3-bromophenyl)cyclohexyl]amino]methyl]-2-pyrrolidinone (CAS number: 2396858-80-1); and
N1-[1-(4-chlorophenyl)cyclohexyl]-1,2-ethanediamine (CAS number: 2031340-15-3).

In one embodiment, the compound is not a compound selected from the group consisting of:
N1,N1-diethyl-N2-methyl-N2-(1-phenylcyclohexyl)ethane-1,2-diamine (CAS number: 2201-45-8);
N1,N1-diethyl-N2-(1-phenylcyclohexyl)ethane-1,2-diamine (CAS number: 2201-53-8);
N-(1-phenylcyclohexyl)-4-morpholineethanamine (CAS number: 2201-54-9);
N2-(3,5-dimethyl-1-phenylcyclohexyl)-N1,N1-diethyl-1,2-ethanediamine (CAS number: 18718-40-6);
N1,N1-diethyl-N2-(3,3,5-trimethyl-1-phenylcyclohexyl)ethane-1,2-diamine (CAS number: 18613-12-2);
N1-(3,5-dimethyl-1-phenylcyclohexyl)-N2,N2-dimethylethane-1,2-diamine (CAS number: 18613-13-3);
N-(3,5-dimethyl-1-phenylcyclohexyl)-1-piperidineethanamine (CAS number: 18613-14-4);
N-(3,3,5-trimethyl-1-phenylcyclohexyl)-1-piperidineethanamine (CAS number: 18613-15-5); and
N-(3,5-dimethyl-1-phenylcyclohexyl)-N-methyl-1-piperidineethanamine (CAS number: 17061-43-7).

In one embodiment, the compound is N-{[(2S)-pyrrolidin-2-yl]methyl}-1-[3-(trifluoromethyl)phenyl]cyclobutan-1-amine. In one embodiment, the compound is N-{[(2R)-pyrrolidin-2-yl]methyl}-1-[3-(trifluoromethyl)phenyl]cyclobutan-1-amine. In one embodiment, the compound is 2-methyl-N1-{1-[3-(trifluoromethyl)phenyl]cyclobutyl}propane-1,2-diamine. In one embodiment, the compound is methyl N-{[(2S)-pyrrolidin-2-yl]methyl}-N-{1-[3-(trifluoromethyl)phenyl]cyclobutyl}carbamate. In one embodiment, the compound is methyl N-{[(2R)-pyrrolidin-2-yl]methyl}-N-{1-[3-(trifluoromethyl)phenyl]cyclobutyl}carbamate. In one embodiment, the compound is methyl N-(2-amino-2-methylpropyl)-N-{1-[3-(trifluoromethyl)phenyl]cyclobutyl}carbamate. In one embodiment, the compound is N1-[1-(3-chlorophenyl)cyclobutyl]-2-methylpropane-1,2-diamine. In one embodiment, the compound is methyl N-[1-(3-chlorophenyl)cyclobutyl]-N-{[(2S)-pyrrolidin-2-yl]methyl}carbamate. In one embodiment, the compound is methyl N-[1-(3-chlorophenyl)cyclobutyl]-N-{[(2R)-pyrrolidin-2-yl]methyl}carbamate. In one embodiment, the compound is N-{[(2S)-pyrrolidin-2-yl]methyl}-1-[3-(trifluoromethoxy)phenyl]cyclobutan-1-amine. In one embodiment, the compound is N-{[(2R)-pyrrolidin-2-yl]methyl}-1-[3-(trifluoromethoxy)phenyl]cyclobutan-1-amine. In one embodiment, the compound is methyl N-{[(2S)-pyrrolidin-2-yl]methyl}-N-{1-[3-(trifluoromethoxy)phenyl]cyclobutyl}carbamate. In one embodiment, the compound is methyl N-{[(2R)-pyrrolidin-2-yl]methyl}-N-{1-[3-(trifluoromethoxy)phenyl]cyclobutyl}carbamate. In one embodiment, the compound is methyl N-(2-amino-2-methylpropyl)-N-{1-[3-(trifluoromethoxy)phenyl]cyclobutyl}carbamate. In one embodiment, the compound is 1-[4-fluoro-3-(trifluoromethyl)phenyl]-N-{[(2S)-pyrrolidin-2-yl]methyl}cyclobutan-1-amine.

In one embodiment, the compound is 1-[4-fluoro-3-(trifluoromethyl)phenyl]-N-{[(2R)-pyrrolidin-2-yl]methyl}cyclobutan-1-amine. In one embodiment, the compound is N1-{1-[4-fluoro-3-(trifluoromethyl)phenyl]cyclobutyl}-2-methylpropane-1,2-diamine. In one embodiment, the compound is methyl N-{1-[4-fluoro-3-(trifluoromethyl)phenyl]cyclobutyl}-N-{[(2S)-pyrrolidin-2-yl]methyl}carbamate. In one embodiment, the compound is methyl N-{1-[4-fluoro-3-(trifluoromethyl)phenyl]cyclobutyl}-N-{[(2R)-pyrrolidin-2-yl]methyl}carbamate. In one embodiment, the compound is methyl N-(2-amino-2-methylpropyl)-N-{1-[4-fluoro-3-(trifluoromethyl)phenyl]cyclobutyl}carbamate. In one embodiment, the compound is 1-[3-fluoro-5-(trifluoromethyl)phenyl]-N-{[(2R)-pyrrolidin-2-yl]methyl}cyclobutan-1-amine.

In one embodiment, the compound is methyl N-{1-[3-fluoro-5-(trifluoromethyl)phenyl]cyclobutyl}-N-{[(2S)-pyrrolidin-2-yl]methyl}carbamate. In one embodiment, the compound is methyl N-{1-[3-fluoro-5-(trifluoromethyl)phenyl]cyclobutyl}-N-{[(2R)-pyrrolidin-2-yl]methyl}carbamate. In one embodiment, the compound is methyl N-(2-amino-2-methylpropyl)-N-{1-[3-fluoro-5-(trifluoromethyl)phenyl]cyclobutyl}carbamate. In one embodiment, the compound is methyl N-{1-[2-fluoro-5-(trifluoromethyl)phenyl]cyclobutyl}-N-{[(2S)-pyrrolidin-2-yl]methyl}carbamate. In one embodiment, the compound is methyl N-[1-(5-chloro-2-fluorophenyl)cyclobutyl]-N-{[(2S)-pyrrolidin-2-yl]methyl}carbamate. In one embodiment, the compound is methyl N-(2-amino-2-methylpropyl)-N-[1-(5-chloro-2-fluorophenyl)cyclobutyl]carbamate. In one embodiment, the compound is 1-(3-cyclopropyl-4-fluorophenyl)-N-{[(2R)-pyrrolidin-2-yl]methyl}cyclobutan-1-amine. In one embodiment, the compound is 1-(3-cyclopropyl-4-fluorophenyl)-N-{[(2S)-pyrrolidin-2-yl]methyl}cyclobutan-1-amine.

Solubility of Compounds

One of the advantages of the compounds of the present invention is that they are more soluble than many other compounds known to modulate potassium channels such as K_{Ca}3.1. The compounds tested in Example 30 have a solubility in pH 7.4 phosphate buffer of 400 to 1700 μM.

Pharmaceutically Acceptable Salts

The chemical compound of the invention may be provided in any form suitable for the intended administration, including pharmaceutically (i.e. physiologically) acceptable salts. Examples of pharmaceutically acceptable addition salts include, without limitation, non-toxic inorganic and organic acid addition salts such as hydrochloride, hydrobromide, nitrate, perchlorate, phosphate, sulphate, formate, acetate, aconate, ascorbate, benzenesulphonate, benzoate, cinnamate, citrate, embonate, enantate, fumarate, glutamate, glycolate, lactate, maleate, malonate, mandelate, methanesulphonate, naphthalene-2-sulphonate, phthalate, salicylate, sorbate, stearate, succinate, tartrate, toluene-p-sulphonate, and the like. Such salts may be formed by procedures well known and described in the art. Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of the compound of the invention include, without limitation, the sodium, the potassium, the calcium, the magnesium, the zinc, the aluminium, the lithium, the choline, the lysinium, and the ammonium salt, and the like, of the compound of the invention containing an anionic group. Such cationic salts may be formed by procedures well known and described in the art. In the context of this invention the “onium salts” of N-containing compounds are also contemplated as pharmaceutically acceptable salts. Preferred “onium salts” include the alkylonium salts, the cycloalkylonium salts, and the cycloalkylalkylonium salts. In one embodiment, the term “pharmaceutically acceptable salt” of a compound designates any “onium” salts of N-containing compounds or any salt of addition of said active principle with a mineral or organic acid among which acetic, hydrochloric, cinnamic, citric, formic, hydrobromic, hydrolodic, hydrofluoric, malonic, methanesulphconic, oxalic, picric, maleic, lactic, nicotinic, phenylacetic, phosphoric, succinic and tartric acid, ammonium, diethylamine, piperazine, nicotinamide, urea, sodium, potassium, calcium, magnesium, zinc, lithium, methylamino, dimethylamino, trimethylamino and tris(hydroxymethyl)aminomethane acid.

Preparation of Compounds

Compounds according to the present invention may be prepared according to any conventional methods of chemical synthesis known by the skilled person, e.g. those described in the working examples. The starting materials for the processes described in the present application are known or may readily be prepared by conventional methods known by the skilled artisan from commercially available chemicals.

The end products of the reactions described herein may be isolated by conventional technique such as extraction, crystallisation, distillation, chromatography etc.

The compounds of this invention may exist in unsolvated as well as in solvated forms with pharmaceutically acceptable solvents such as water, ethanol and the like. In general, the solvated forms are considered equivalent to the unsolvated forms for the purposes of this invention.

Pharmaceutical Compositions

The present invention also relates to a pharmaceutical composition comprising, for example as an active ingredient, a pharmaceutically effective amount of a compound as disclosed herein. In one embodiment, said pharmaceutical composition comprises a therapeutically effective amount of the compound as disclosed herein or a pharmaceutically acceptable salt thereof, together with at least one pharmaceutically acceptable carrier, excipient or diluent.

While a compound as disclosed herein for use in therapy may be administered in the form of the raw chemical compound, it is preferred to introduce the active ingredient, optionally in the form of a pharmaceutically acceptable salt, in a pharmaceutical composition together with one or more adjuvants, excipients, carriers, buffers, diluents, and/or other customary pharmaceutical auxiliaries.

In one embodiment, the invention provides pharmaceutical compositions comprising a compounds disclosed herein or a pharmaceutically acceptable salt thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof. Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

A compound as disclosed herein, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. A compound as disclosed herein can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

Unlike many of the other known KCa3.1 inhibitors, the compounds of the present invention has a high solubility in aqueous medium, which makes them suitable for liquid drug administration, such as intravenous or infusion administration.

For preparing pharmaceutical compositions from a compound as disclosed herein, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

The pharmaceutical preparations may be in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

A therapeutically effective dose refers to that amount of active ingredient, which ameliorates the symptoms or condition. Therapeutic efficacy and toxicity, e.g. ED₅₀, may be determined by standard pharmacological procedures in cell cultures or experimental animals. The dose ratio between therapeutic and toxic effects is the therapeutic index and may be expressed by ratio between plasma levels resulting in therapeutic effects and plasma ratios resulting in toxic effects. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 10000 mg of active ingredient per individual dose, such as 0.5 to 2000 mg, preferably of from about 1 to about 1000 mg, most preferred of from about 10 to about 500 mg, are suitable for therapeutic treatments. The active ingredient may be administered in one or several doses per day.

Biological Activity

Compounds of the present invention are active as potassium channel modulators. The compounds of the present invention tested in example 31 all inhibit K_{Ca}3.1.

Method of Treatment

Being modulators of potassium ion channels, such as K_{Ca}3.1, the compounds of the present invention are of use in the treatment of diseases and disorders of a living body, including human. In one embodiment, the term “treatment” also includes prevention, and/or alleviation of diseases and disorders. In one aspect, the compound as described herein is for use in medicine. In one aspect, the present invention relates to a method for treatment of IBD, hereditary xerocytosis or ARDS comprising administration of a compound as described herein, or a pharmaceutical composition comprising said compound, to a subject in need thereof.

Inflammatory Bowel Diseases (IBD)

Inflammatory bowel disease (IBD) is a chronic autoimmune disease affecting the gastrointestinal tract with symptoms of abdominal pain, vomiting, diarrhoea, hematochezia, and weight loss. IBD comes in two main forms, ulcerative colitis (UC) and Crohn's disease (CD). UC exclusively affects the colon and rectum, whereas CD may affect the entire gastrointestinal tract. Histologically UC is characterized by extended mucosal inflammation in contrast to CD, where deep punctuate lesions affect all layers of the intestinal wall. It is estimated that approximately 2.5 million patients are diagnosed with IBD (1 million with colitis and 1.5 million with Crohns patients) in the industrialized world (USA, Japan; 5 major EU countries). The incidences are increasing, especially in newly industrialized countries, possibly related to changes in lifestyle.

Currently used anti-IBD drugs are anti-inflammatory (5-ASA's, steroids), generally immune dampening (azathioprine, 6-mercaptopurine), or biological single cytokine/integrine neutralizing agents (eg. infleximap, ustekinomap, vedolizumap). Despite these options and carefully optimized clinical procedures, patients still face rounds of gut-shortening surgeries (many Crohns patients experience at least one surgery in their lifetime), and colitis patients may develop proctitis after colectomy. Suboptimal medical disease control with respect to maintaining long-term remission, to fight flare ups, and especially avoiding development of irreversible structural changes due to irresolvable gut fibrosis, represents a serious unmet need for IBD patients.

Many of the drugs used to treat IBD today are connected with side effects. For example, side effects of steroids include increased susceptibility to infection; and 5-aminosalicylic acids, such as in the form of sulphasalazine, are associated with a significant proportion of non-responders among UC patients, decreased kidney function as well as high and frequent doses, which elicit poor compliance. Drawbacks for TNF-alpha inhibitor infliximab are include high cost, inconvenience of application (injections), waning of efficacy and elicitation of increased risk of infection as a result of their immunosuppressive characteristic; and immunomodulators such asazathioprine, 6-mercaptopurine and methotrexate increase the risk for infections and for some types of cancer, as well as being liver toxic. Thus, there is still a major unmet need for new treatments of inflammatory bowel diseases.

K_{Ca}3.1 as a Target for IBD

T cells play an important role in IBD, and IBD processes (immune cell proliferation, homing, and cytokine release), excessive fibroblast-mediated collagen secretion can lead to fibrosis that causes strictures and intestinal obstructions, and excess water transport across the epithelia can lead to diarrhoea. All these pathological processes can be dampened by K_{Ca}3.1 inhibition.

As demonstrated herein, the compounds of the present invention inhibit K_{Ca}3.1, and thus, in one aspect, the present invention relates to a compound as described herein for use in the treatment, alleviation and/or prevention of inflammatory bowel disease (IBD). In one embodiment, said IBD is colitis, such as ulcerative colitis (UC). In one embodiment, said IBD is Crohn's disease (CD).

Hereditary Xerocytosis

Hereditary xerocytosis, also known as dehydrated stomatocytosis, is characterized by increased fragility and haemolysis of erythrocytes, resulting in a fully compensated or mild to severe anaemia. Increased reticulocyte formation (to compensate for erythrocyte loss), ion-overload and jaundice (resulting from the increased break-down of haemoglobin) are characteristic in adults. New-borns may suffer from transient edema/ascites, which in rare cases may develop to life-threatening hydrops fetalis. The disease is very heterogeneous but is classically identified from a combination of clinical signs, such as fatigue, enlarged spleen, gall stones, thrombosis events, and pulmonary hypertension. Microscopic examination may reveal erythrocytes with abnormal shapes and analysis of haematology parameters reveal shrunken erythrocytes due to salt and water loss. The ethology of hereditary xerocytosis has long been known to differ radically from other hereditary anaemias, such as the haemoglobinopathies (eg. sickle cell anemia and the thalassemia diseases), or glycolytic enzymopathies (eg. glucose-6-phosphate deficiency), in that it is due to a primary membrane permeability defect. The molecular targets involved in this defect have just recently been identified.

K_{Ca}3.1 as a Target for Hereditary Xerocytosis

Recent years of scientific investigations have shown that hereditary xerocytosis is due to gain-of-function mutations in either Piezo1 or KCNN4, the gene encoding K_{Ca}3.1. Both mutations essentially result in the same phenotype: In the case of Piezo1 mutations Ca²⁺ enters the erythrocyte through the constantly open channel, thus activating K_{Ca}3.1 resulting in permanently dehydrated erythrocytes; in the case of KCNN4 mutations K_{Ca}3.1 are constitutively open thereby governing erythrocyte dehydration even in the absence of a Ca²⁺-signal from the Piezo1 channel. The clear definition of the genes and mutations responsible for hereditary xerocytosis, allows easy diagnostics of which patients will benefit from the treatment and which should not be treated.

Inhibition of the erythrocyte K_{Ca}3.1 channel will counteract unintentional dehydration and presumably prevent haemolysis of xerocytosis erythrocytes and thereby improve the clinical condition of patients. Importantly, the binding site for K_{Ca}3.1 inhibitors do not overlap with the known gain-of-function mutations in K_{Ca}3.1. This pinpoints K_{Ca}3.1 as a pivotal target for all known causes of hereditary xerocytosis.

The compounds of the present invention inhibit K_{Ca}3.1. Hence, in one aspect, the present invention relates to a compound described herein for use in the treatment, alleviation and/or prevention of hereditary xerocytosis. Hereditary xerocytosis is one of the most frequent variant of hereditary stomatocytoses, a group of rare disorders characterized by a leak of monovalent cations such as K⁺ from the red blood cells (RBCs).

Acute Respiratory Distress Syndrome (ARDS)

Acute respiratory distress syndrome is a serious and often lethal complication to lung infections, as caused for example by SARS, MERS, or Covid-19 vira. The infections can lead to global lung inflammation, which widens the ultrathin barriers between the air-filled alveoli and the blood-filled alveolar vessels and fills-up the alveoli with liguid, and thereby hampers the life-essential oxygen/carbondioxide gas exchange between lung and blood. ARDS is thus a complex condition that involve both components of the immune system as well as the air/blood barrier function. Since there are currently no medical treatments that specifically interfere with ARDS (general immune dampening treatments by steroids are not effective), the only options for patients is medical ventilator treatment at an intensive care unit.

KCa3.1 as a Target for ARDS.

Since the KCa3.1 channel is expressed in both the epithelia and endothelia as well as in the inflammatory cells, such as neutrophils, that participate in lung inflammation, inhibition of KCa3.1 can dampen both the basic inflammation and possibly also protect the air/blood barrier function. Experiments with a mouse model of ARDS have recently shown that KCa3.1 knock-out mice have improved gas exchange, and the improvement was also demonstrated by treatment with the classical KCa3.1 inhibitors senicapoc and TRAM-34. In the clinical situation with patients on medical ventilation, oral drug administration is not optimal, whereas intravenous bolus or infusion administration is preferred. Classical KCa3.1 inhibitors like the triarylmethanes (exemplified by senicapoc and TRAM-34) have extremely low water solubility, which makes IV-formulations very challenging. The same drawbacks apply to known KCa3.1 inhibitors based on other chemical scaffolds.

The compounds of the present invention inhibit K_{Ca}3.1. Further, the compounds of the present invention has a high solubility in aqueous medium. Hence, the compounds of the present invention are highly suitable for use in treatment of ARDS. Thus, in one aspect, the present invention relates to a compound as described herein for use in the treatment, alleviation and/or prevention of ARDS.

Items

1. A compound of formula (I):

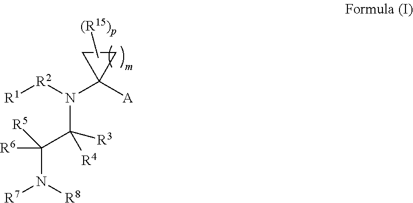

wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R³ is H, a bond, —OH, or C₁₋₈ alkyl, wherein one or more methylene group optionally and individually is replaced by —O— and/or substituted with ═O;
R¹⁵ is individually selected from the group consisting of C₁₋₃ alkyl, —OH, —CN, and —F;
anyone of R³, R⁴, R⁵, R⁶, R⁷, and R³ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₃ alkyl, —OC₁₋₃ alkyl, —C₃₋₇ cycloalkyl, —OC₃₋₇ cycloalkyl, —CN, NO₂, —SO₂CH₃, and —SF₅;
X is halogen;
m is an integer of 1 to 4; and
p is an integer of 0 to 10;
or a pharmaceutically acceptable salt thereof.

2. The compound according to item 1, with the proviso that p is not 0 when m is 1.

3. The compound according to any one of the preceding items, wherein the compound is of formula (II):

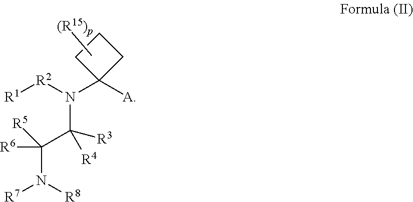

4. The compound according to any one of the preceding items, wherein the compound is of formula (III):

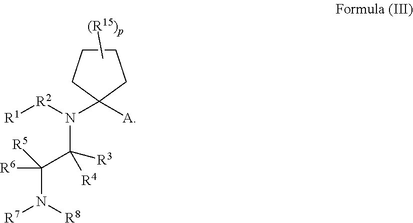

5. The compound according to any one of the preceding items, wherein the compound is of formula (IV):

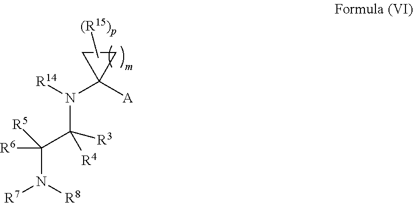

wherein
R¹⁴ is selected from the group consisting of —C(O)—C₁₋₈ alkyl; —C(O)—O—C₁₋₈ alkyl; —C₂₋₈ alkyl; —H and —S(O)₂—C₁₋₈ alkyl;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R⁸ is H, a bond, —OH, or C₁₋₈ alkyl, wherein one or more methylene group optionally and individually is replaced by —O— and/or substituted with ═O;
R¹⁵ is individually selected from the group consisting of C₁₋₃ alkyl, —OH, —CN, and —F;
anyone of R³, R⁴, R⁵, R⁶, R⁷, and R⁸ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅; and
X is halogen;
m is an integer of 1 to 4; and
p is an integer of 0 to 10;
or a pharmaceutically acceptable salt thereof.

6. The compound according to any one of the preceding items, wherein the compound is of formula (V):

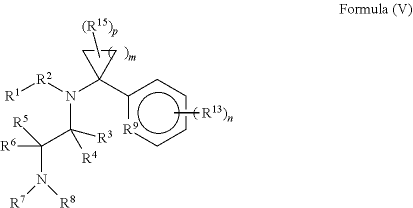

wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R³ is H, a bond, —OH, or C₁₋₈ alkyl, wherein one or more methylene group optionally and individually is replaced by —O— and/or substituted with ═O;
R⁹ is —C(H)—, —N—, or —C(R¹³)—;
R¹³ is individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
R¹⁵ is individually selected from the group consisting of C₁₋₂ alkyl, —OH, —CN, and —F;
anyone of R³, R⁴, R⁵, R⁶, R⁷, and R³ optionally is linked together to form a ring;
X is halogen;
n is an integer of 0 to 4;
m is an integer of 1 to 4; and
p is an integer of 0 to 10;
or a pharmaceutically acceptable salt thereof.

7. The compound according to any one of the preceding items, wherein the compound is of formula (VI):

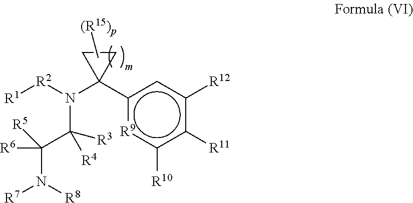

wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R³ is H, a bond, —OH, or C₁₋₈ alkyl, wherein one or more methylene group optionally and individually is replaced by —O— and/or substituted with ═O; anyone of R³, R⁴, R⁵, R⁶, R⁷, and R³ optionally is linked together to form a ring;
R⁹ is —C(H)—, —N—, or —C(R¹³)—;
R¹⁰, R¹¹, R¹², and R¹³ are individually selected from the group consisting of H, halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
R¹⁵ is individually selected from the group consisting of C₁₋₃ alkyl, —OH, —CN, and —F;
X is halogen;
m is an integer of 1 to 4; and
p is an integer of 0 to 10;
or a pharmaceutically acceptable salt thereof.

8. The compound according to any one of the preceding items, wherein the compound is of formula (VI):

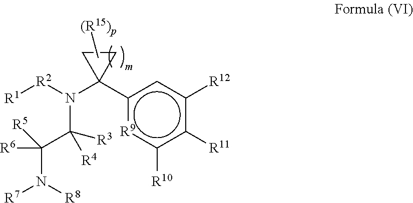

wherein
R¹ is —OC₁₋₈alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₃ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R⁸ is H, a bond, —OH, or C₁₋₈ alkyl, wherein one or more methylene group optionally and individually is replaced by —O— and/or substituted with ═O; anyone of R³, R⁴, R⁵, R⁶, R⁷, and R³ optionally is linked together to form a ring;
R⁹ is —C(H)— or —N—;
R¹⁰ is H or halogen;
R¹¹ is H or halogen;
R¹² is —CX₃, —OCX₃, H, halogen, —C₁₋₈ alkyl, or —C₃₋₇ cycloalkyl;
R¹⁵ is individually selected from the group consisting of C₁₋₃ alkyl, —OH, —CN, and —F
X is halogen;
m is an integer of 1 to 4; and
p is an integer of 0 to 10;
or a pharmaceutically acceptable salt thereof.

9. The compound according to any one of the preceding items, wherein the compound is of formula (VII):

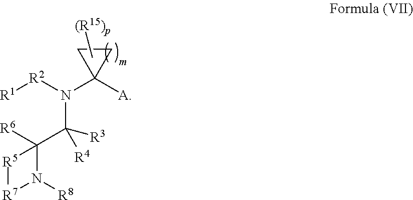

10. The compound according to any one of the preceding items, wherein the compound is of formula (VIII):

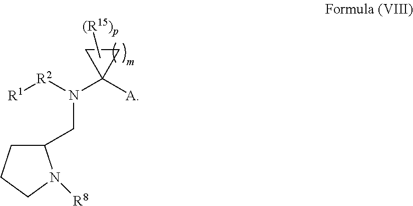

11. The compound according to any one of the preceding items, wherein the compound is of formula (IX):

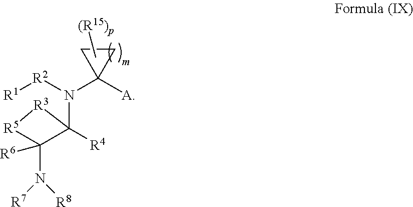

12. The compound according to any one of the preceding items, wherein the compound is of formula (X):

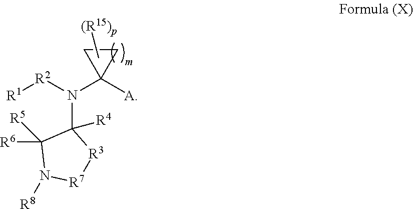

13. The compound according to any one of the preceding items, wherein the compound is of formula (XI):

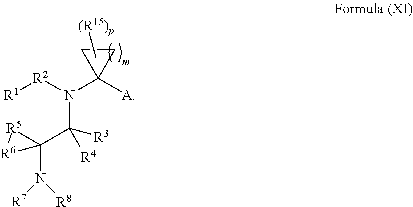

14. The compound according to any one of the preceding items, wherein the compound is of formula (XIV):

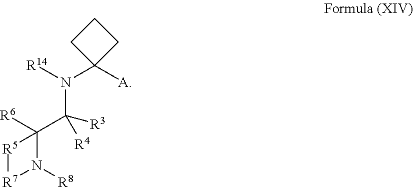

15. The compound according to any one of the preceding items, wherein the compound is of formula (XV):

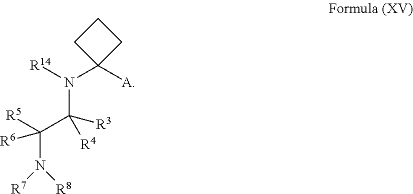

16. The compound according to any one of the preceding items, wherein m is 2.

17. The compound according to any one of the preceding items, wherein m is 3.

18. The compound according to any one of the preceding items, wherein m is 1 and p is an integer of 1 to 4.

19. The compound according to any one of the preceding items, wherein p is 0.

20. The compound according to any one of the preceding items, wherein A is a moiety of formula (XII):

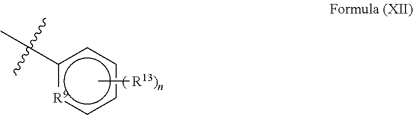

wherein
R⁹ is —C(H)—, —N—, or —C(R¹³)—;
R¹³ is individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
n is an integer of 0 to 4; and
X is halogen.

21. The compound according to any one of the preceding items, wherein A is a moiety of formula (XIII):

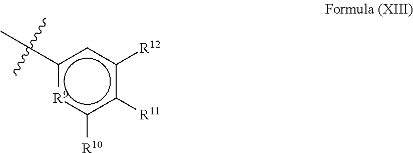

wherein
R⁹ is —C(H)—, —N—, or —C(R¹³)—;
R¹⁰, R¹¹, R¹², and R¹³ are individually selected from the group consisting of H, halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅; and
X is halogen.

22. The compound according to any one of the preceding items, wherein
R⁹ is —C(H)— or —N—;
R¹⁰ is H or halogen;
R¹¹ is H or halogen;
R¹² is —CX₃, —OCX₃, H, halogen, —C₁₋₄ alkyl, or —C₃₋₅ cycloalkyl; and
X is halogen.

23. The compound according to any one of the preceding items, wherein R¹ is —OC₁₋₈ alkyl, such as —OC₁₋₇ alkyl, such as —OC₁— alkyl, such as —OC₁₋₅ alkyl, such as —OC₁₋₄ alkyl, such as —OC₁₋₃ alkyl, such as —OC₁₋₂ alkyl, such as —OC₁ alkyl.

24. The compound according to any one of the preceding items, wherein R¹ is —C₁₋₈ alkyl, such as —C₁₋₇ alkyl, such as —C₁₋₆ alkyl, such as —C₁₋₅ alkyl, such as —C₁₋₄ alkyl, such as —C₁₋₃ alkyl, such as —C₁₋₂ alkyl, such as —C₁ alkyl.

25. The compound according to any one of the preceding items, wherein R¹ is —C₁₋₃ alkyl substituted with —OH.

26. The compound according to any one of the preceding items, wherein R¹ is —H.

27. The compound according to any one of the preceding items, wherein R² is a bond.

28. The compound according to any one of the preceding items, wherein R² is-C(O)—.

29. The compound according to any one of the preceding items, wherein R² is —C(H)₂—.

30. The compound according to any one of the preceding items, wherein R² is —S(O)₂—.

31. The compound according to any one of the preceding items, wherein R² is —C(O)— and R¹ is —OC₁₋₄ alkyl.

32. The compound according to any one of the preceding items, wherein R² is —C(O)— and R¹ is —OC₁₋₃ alkyl.

33. The compound according to any one of the preceding items, wherein R² is a bond and R¹ is C₃₋₄ alkyl.

34. The compound according to any one of the preceding items, wherein R¹ is —OC₁— alkyl, or —C₁₋₃ alkyl, optionally substituted with —OH, and R² is a bond, —C(O)—, —S(O)₂—, or —C(H)₂—.

35. The compound according to any one of the preceding items, wherein —R¹-R² is not H.

36. The compound according to any one of the preceding items, wherein —R²-R¹ is —R¹⁴, and R¹⁴ is selected from the group consisting of —C(O)—C₁₋₃ alkyl; —C(O)—O—C₁₋₃ alkyl; —C₂₋₈ alkyl; —H and —S(O)₂—C₁₋₈ alkyl.

37. The compound according to any one of the preceding items, wherein —R²-R¹ is —R¹⁴, and R¹⁴ is selected from the group consisting of —C(O)—C₁₋₃ alkyl; —C(O)—O—C₁₋₈ alkyl; —C₂₋₈ alkyl; and —S(O)₂—C₁₋₈ alkyl.

38. The compound according to any one of the preceding items, wherein R¹⁴ is —C(O)—C₁₋₈ alkyl, such as R¹⁴ is —C(O)—C₁₋₃ alkyl, such as R¹⁴ is —C(O)—C₃ alkyl, such as —C(O)-cyclopropyl.

39. The compound according to any one of the preceding items, wherein R¹⁴ is —C(O)—O—C₁₋₃ alkyl, such as, R¹⁴ is —C(O)—OC₁₋₃ alkyl, such as R¹⁴ is selected from the group consisting of —C(O)—OCH₃, —C(O)—OCH₂CH₃, —OCH₂(CH₃)₂ and —O— cyclopropyl.

40. The compound according to any one of the preceding items, wherein R¹⁴ is —C₂₋₈ alkyl, such as C₃₋₄ alkyl.

41. The compound according to any one of the preceding items, wherein R¹⁴ is —C(H)₂—C₃₋₇ cycloalkyl, such as —C(H)₂-cyclopropyl or —C(H)₂-cyclobutyl. In one

42. The compound according to any one of the preceding items, wherein R¹⁴ is —C₃₋₇ cycloalkyl, such as— cyclopropyl or— cyclobutyl.

43. The compound according to any one of the preceding items, wherein R¹⁴ is —C₂₋₈ alkyl, such as C₃₋₄ alkyl, substituted with one or more —OH, such as R¹⁴ is isopropyl substituted with —OH.

44. The compound according to any one of the preceding items, wherein R¹⁴ is —H.

45. The compound according to any one of the preceding items, wherein R¹⁴ is —S(O)₂—C₁₋₈ alkyl, such as R¹⁴ is —S(O)₂—CH₃.

46. The compound according to any one of the preceding items, wherein R¹⁴ is not —CH₃.

47. The compound according to any one of the preceding items, wherein R³ and R⁴ are —H, R⁵ and R⁶ are methyl, and R⁷ and R⁸ are —H.

48. The compound according to any one of the preceding items, wherein R³ is H.

49. The compound according to any one of the preceding items, wherein R³ is C₁₋₅ alkyl, such as C₁₋₄ alkyl, such as C₁₋₃ alkyl, such as C₁₋₂ alkyl, such as Ci alkyl.

50. The compound according to any one of the preceding items, wherein R⁴ is H.

51. The compound according to any one of the preceding items, wherein R⁴ is C₁₋₅ alkyl, such as C₁₋₄ alkyl, such as C₁₋₃ alkyl, such as C₁₋₂ alkyl, such as C₁ alkyl.

52. The compound according to any one of the preceding items, wherein R³ and R⁴ are H.

53. The compound according to any one of the preceding items, wherein R⁵ is H.

54. The compound according to any one of the preceding items, wherein R⁵ is C₁₋₈ alkyl, such as C₁₋₇ alkyl, such as C₁₋₆ alkyl, such as C₁₋₅ alkyl, such as C₁₋₄ alkyl, such as C₁₋₃ alkyl, such as C₁₋₂ alkyl, such as C₁ alkyl.

55. The compound according to any one of the preceding items, wherein R⁶ is H.

56. The compound according to any one of the preceding items, wherein R⁶ is C₁₋₈ alkyl, such as C₁₋₇ alkyl, such as C₁₋₆ alkyl, such as C₁₋₅ alkyl, such as C₁₋₄ alkyl, such as C₁₋₃ alkyl, such as C₁₋₂ alkyl, such as C₁ alkyl.

57. The compound according to any one of the preceding items, wherein R⁵ and R⁶ are H.

58. The compound according to any one of the preceding items, wherein R⁵ and R⁶ are —CH₃.

59. The compound according to any one of the preceding items, wherein R⁵ and R⁶ are linked together to form a ring.

60. The compound according to any one of the preceding items, wherein R⁷ is C₁₋₃ alkyl, such as C₁₋₇ alkyl, such as C₁₋₆ alkyl, such as C₁₋₅ alkyl, such as C₁₋₄ alkyl, such as C₁₋₃ alkyl, such as C₁₋₂ alkyl, such as C₁ alkyl.

61. The compound according to any one of the preceding items, wherein R⁷ is H.

62. The compound according to any one of the preceding items, wherein R⁷ is —C(O)—O—CH₃ or —C(O)—CH₃.

63. The compound according to any one of the preceding items, wherein R⁸ is C₁₋₃ alkyl, such as C₁₋₇ alkyl, such as C₁₋₆ alkyl, such as C₁₋₅ alkyl, such as C₁₋₄ alkyl, such as C₁₋₃ alkyl, such as C₁₋₂ alkyl, such as C₁ alkyl.

64. The compound according to any one of the preceding items, wherein R⁷ is selected from the group consisting of H, a bond, —OH, or C₁₋₃ alkyl, wherein one or more methylene group optionally and individually is replaced by —O—; and R⁸ is selected from the group consisting of H, a bond, —OH, or C₁₋₃ alkyl, wherein one or more methylene group optionally and individually is replaced by —O—.

65. The compound according to any one of the preceding items, wherein R⁸ is H.

66. The compound according to any one of the preceding items, wherein R⁵ or R⁶ is linked to R⁷ or R⁸ to form a ring, such as R⁵ is linked to R⁷. 67. The compound according to any one of the preceding items, wherein R⁵ is linked to R⁷ to form a four-, five- or six membered ring.

68. The compound according to any one of the preceding items, wherein R³ or R⁴ is linked to R⁵ or R⁶ to form a ring, such as R³ is linked to R⁵.

69. The compound for use according to any one of the preceding items, wherein R³ is linked to R⁵ to form a four or five membered ring.

70. The compound according to any one of the preceding items, wherein R³ or R⁴ is linked together to R⁷ or R⁸ to form a ring, such as R³ is linked to R⁷.

71. The compound for use according to any one of the preceding items, wherein R³ is linked to R⁷ to form a four membered ring.

72. The compound according to any one of the preceding items, wherein R⁹ is —C(H)—.

73. The compound according to any one of the preceding items, wherein R⁹ is —C(F)—.

74. The compound according to any one of the preceding items, wherein R¹⁰, R¹¹ and R¹² are individually selected from the group consisting of H, halogen, —CX₃, —OCX₃, —C₁₋₈ alkyl, and —C₃₋₇ cycloalkyl.

75. The compound according to any one of the preceding items, wherein R¹⁰, R¹¹ and R¹² are individually selected from the group consisting of H, halogen, —CX₃, and —OCX₃. 76. The compound according to any one of the preceding items, wherein R¹⁰ is H.

77. The compound according to any one of the preceding items, wherein R¹⁰ is F.

78. The compound according to any one of the preceding items, wherein R¹⁰ is Cl.

79. The compound according to any one of the preceding items, wherein R¹¹ is F.

80. The compound according to any one of the preceding items, wherein R¹¹ is H.

81. The compound according to any one of the preceding items, wherein R¹² is —CF₃.

82. The compound according to any one of the preceding items, wherein R¹² is —OCF₃.

83. The compound according to any one of the preceding items, wherein R¹² is F, Cl or Br.

84. The compound according to any one of the preceding items, wherein R¹² is —C₁₋₈ alkyl, such as —C₁ alkyl, such as —C₂ alkyl, such as —C₃ alkyl.

85. The compound according to any one of the preceding items, wherein, R¹² is —C₃₋₇ cycloalkyl, such as —C₃ cycloalkyl, such as —C₃ cycloalkyl, such as —C₅ cycloalkyl.

86. The compound according to any one of the preceding items, wherein R¹¹ is F and R¹² is —CF₃.

87. The compound according to any one of the preceding items, wherein R⁹ is —C(H)—, R¹⁰ is H, R¹¹ is F and R¹² is —CF₃ or —OCF₃.

88. The compound according to any one of the preceding items, wherein R⁹ is —C(H)—, R¹⁰ is H, R¹¹ is F and R¹² is —CF₃.

89. The compound according to any one of the preceding items, wherein R⁹ is —C(H)—, R¹⁰ is H, R¹¹ is H and R¹² is —CF₃.

90. The compound according to any one of the preceding items, wherein R⁹ is —C(H)—, R¹⁰ is F, R¹¹ is H and R¹² is —CF₃.

91. The compound according to any one of the preceding items, wherein R⁹ is —C(F)—, R¹⁰ is H, R¹¹ is H and R¹² is —CF₃.

92. The compound according to any one of the preceding items, wherein R⁹ is —C(H)—, R¹⁰ is H, R¹¹ is F and R¹² is —OCF₃.

93. The compound according to any one of the preceding items, wherein R⁹ is —C(H)—, R¹⁰ is H, R¹¹ is H and R¹² is —OCF₃.

94. The compound according to any one of the preceding items, wherein R⁹ is —C(H)—, R¹⁰ is H, R¹¹ is H and R¹² is halogen.

95. The compound according to any one of the preceding items, wherein R⁹ is —C(F)—, R¹⁰ is H, R¹¹ is H and R¹² is halogen.

96. The compound according to any one of the preceding items, wherein R⁹ is —C(H)—, R¹⁰ is H, R¹¹ is F and R¹² is —C₃ cycloalkyl.

97. The compound according to any one of the preceding items, wherein the compound is the (S)-enantiomer.

98. The compound according to any one of the preceding items, wherein the compound is the (R)-enantiomer.

99. The compound according to any one of the preceding items, wherein —R¹-R² is not—CH₃.

100. The compound according to any one of the preceding items, wherein no more than two of R¹⁰, R¹¹ and R¹² are H.

101. The compound according to any one of the preceding items, wherein no more than one of R¹⁰, R¹¹ and R¹² are H.

102. The compound according to any one of the preceding items, wherein when R¹¹ and R¹² are H, then R¹⁰ is halogen.

103. The compound according to any one of the preceding items, wherein no more than five of R³, R⁴, R⁵, R⁶, R⁷ and R⁸ are H.

104. The compound according to any one of the preceding items, wherein when R³, R⁴, R⁵, R⁶, R⁷ and R⁸ are H, then no more than two of R¹⁰, R¹¹ and R¹² are H.

105. The compound according to any one of the preceding items, wherein when R³, R⁴, R⁵, R⁶, R⁷ and R⁸ are H, then A is substituted with at least two substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅.

106. The compound according to any one of the preceding items, wherein when R³, R⁴, R⁵, R⁶, R⁷ and R⁸ are H, then —R¹-R² is not H.

107. The compound according to any one of the preceding items, wherein when R³, R⁴, R⁵, R⁶, R⁷ and R⁸ are H, then —R¹⁴ is not H.

108. The compound according to any one of the preceding items, wherein when the compound is of formula (VII), then R³ and R⁴ are H.

109. The compound according to any one of the preceding items, wherein when the compound is of formula (VII) and R³ is H, then R⁴ is not—CH₃.

110. The compound according to any one of the preceding items, wherein when the compound is of formula (VII) and R⁸ is H, then R⁷ is selected from the group consisting of H, a bond, —OH, or C₁₋₈ alkyl, wherein one or more methylene group optionally and individually is replaced by —O—.

111. The compound according to any one of the preceding items, wherein when the compound is of formula (X) and —R³-R⁷ is —CH₂—, then —R—R² is not H.

112. The compound according to any one of the preceding items, wherein when the compound is of formula (X) and —R³-R⁷ is —CH₂—, then A is substituted with at least one substituent R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅.

113. The compound according to any one of the preceding items, wherein the compound is not a compound selected from the group consisting of:
(2R)—N-[1-(4-fluorophenyl)cyclobutyl]-α-methyl-2-pyrrolidinemethanamine;
(2S)—N-[1-(4-fluorophenyl)cyclobutyl]-α-methyl-2-pyrrolidinemethanamine;
N1-[1-(3-bromophenyl)cyclobutyl]-1,2-ethanediamine;
N1-[1-(4-fluorophenyl)cyclobutyl]-1,2-ethanediamine;
N1-[1-(4-chlorophenyl)cyclobutyl]-1,2-ethanediamine;
N-(1-phenylcyclobutyl)-3-azetidinamine;
N1-[1-(4-bromophenyl)cyclobutyl]-1,2-ethanediamine;
(5S)-5-[[[1-(4-fluorophenyl)cyclopentyl]amino]methyl]-2-pyrrolidinone;
5-[[[1-(4-fluorophenyl)cyclopentyl]amino]methyl]-2-pyrrolidinone;
N1-[1-(4-fluorophenyl)cyclopentyl]-1,2-ethanediamine;
N1-[1-(3-methylphenyl)cyclopentyl]-1,2-ethanediamine;
(5S)-5-[[[1-(4-chlorophenyl)cyclohexyl]amino]methyl]-2-pyrrolidinone;
5-[[[1-(4-chlorophenyl)cyclohexyl]amino]methyl]-2-pyrrolidinone;
(5S)-5-[[[1-(4-bromophenyl)cyclohexyl]amino]methyl]-2-pyrrolidinone;
5-[[[1-(4-bromophenyl)cyclohexyl]amino]methyl]-2-pyrrolidinone;
(5S)-5-[[[1-(2-bromophenyl)cyclohexyl]amino]methyl]-2-pyrrolidinone;
5-[[[1-(2-bromophenyl)cyclohexyl]amino]methyl]-2-pyrrolidinone;
(5S)-5-[[[1-(3-bromophenyl)cyclohexyl]amino]methyl]-2-pyrrolidinone;
5-[[[1-(3-bromophenyl)cyclohexyl]amino]methyl]-2-pyrrolidinone; and
N1-[1-(4-chlorophenyl)cyclohexyl]-1,2-ethanediamine.

114. The compound according to any one of the preceding items, wherein the compound is not a compound selected from the group consisting of:
N1,N1-diethyl-N2-methyl-N2-(1-phenylcyclohexyl)ethane-1,2-diamine;
N1,N1-diethyl-N2-(1-phenylcyclohexyl)ethane-1,2-diamine;
N-(1-phenylcyclohexyl)-4-morpholineethanamine;
N2-(3,5-dimethyl-1-phenylcyclohexyl)-N1,N1-diethyl-1,2-ethanediamine;
N1,N1-diethyl-N2-(3,3,5-trimethyl-1-phenylcyclohexyl)ethane-1,2-diamine;
N1-(3,5-dimethyl-1-phenylcyclohexyl)-N2,N2-dimethylethane-1,2-diamine;
N-(3,5-dimethyl-1-phenylcyclohexyl)-1-piperidineethanamine;
N-(3,3,5-trimethyl-1-phenylcyclohexyl)-1-piperidineethanamine (CAS number: 18613-15-5); and
N-(3,5-dimethyl-1-phenylcyclohexyl)-N-methyl-1-piperidineethanamine.

115. The compound according to any one of the preceding items, wherein the compound is selected from the group consisting of:
a. N-{[(2S)-pyrrolidin-2-yl]methyl}-1-[3-(trifluoromethyl)phenyl]cyclobutan-1-amine;
b. N-{[(2R)-pyrrolidin-2-yl]methyl}-1-[3-(trifluoromethyl)phenyl]cyclobutan-1-amine;
c. 2-methyl-N1-{1-[3-(trifluoromethyl)phenyl]cyclobutyl}propane-1,2-diamine;
d. methyl N-{[(2S)-pyrrolidin-2-yl]methyl}-N-{1-[3-(trifluoromethyl)phenyl]cyclobutyl}carbamate;
e. methyl N-{[(2R)-pyrrolidin-2-yl]methyl}-N-{1-[3-(trifluoromethyl)phenyl]cyclobutyl}carbamate;
f. methyl N-(2-amino-2-methylpropyl)-N-{1-[3-(trifluoromethyl)phenyl]cyclobutyl}carbamate;
g. N1-[1-(3-chlorophenyl)cyclobutyl]-2-methylpropane-1,2-diamine;
h. methyl N-[1-(3-chlorophenyl)cyclobutyl]-N-{[(2S)-pyrrolidin-2-yl]methyl}carbamate;
i. methyl N-[1-(3-chlorophenyl)cyclobutyl]-N-{[(2R)-pyrrolidin-2-yl]methyl}carbamate;
j. N-{[(2S)-pyrrolidin-2-yl]methyl}-1-[3-(trifluoromethoxy)phenyl]cyclobutan-1-amine;
k. N-{[(2R)-pyrrolidin-2-yl]methyl}-1-[3-(trifluoromethoxy)phenyl]cyclobutan-1-amine;
I. methyl N-{[(2S)-pyrrolidin-2-yl]methyl}-N-{1-[3-(trifluoromethoxy)phenyl]cyclobutyl}carbamate;
m. methyl N-{[(2R)-pyrrolidin-2-yl]methyl}-N-{1-[3-(trifluoromethoxy)phenyl]cyclobutyl}carbamate;
n. methyl N-(2-amino-2-methylpropyl)-N-{1-[3-(trifluoromethoxy)phenyl]cyclobutyl}carbamate;
o. 1-[4-fluoro-3-(trifluoromethyl)phenyl]-N-{[(2S)-pyrrolidin-2-yl]methyl}cyclobutan-1-amine;
p. 1-[4-fluoro-3-(trifluoromethyl)phenyl]-N-{[(2R)-pyrrolidin-2-yl]methyl}cyclobutan-1-amine;
q. N1-{1-[4-fluoro-3-(trifluoromethyl)phenyl]cyclobutyl}-2-methylpropane-1,2-diamine;
r. methyl N-{1-[4-fluoro-3-(trifluoromethyl)phenyl]cyclobutyl}-N-{[(2S)-pyrrolidin-2-yl]methyl}carbamate;
s. methyl N-{1-[4-fluoro-3-(trifluoromethyl)phenyl]cyclobutyl}-N-{[(2R)-pyrrolidin-2-yl]methyl}carbamate;
t. methyl N-(2-amino-2-methylpropyl)-N-{1-[4-fluoro-3-(trifluoromethyl)phenyl]cyclobutyl}carbamate;
u. 1-[3-fluoro-5-(trifluoromethyl)phenyl]-N-{[(2R)-pyrrolidin-2-yl]methyl}cyclobutan-1-amine;
v. methyl N-{1-[3-fluoro-5-(trifluoromethyl)phenyl]cyclobutyl}-N-{[(2S)-pyrrolidin-2-yl]methyl}carbamate;
w. methyl N-{1-[3-fluoro-5-(trifluoromethyl)phenyl]cyclobutyl}-N-{[(2R)-pyrrolidin-2-yl]methyl}carbamate;
x. methyl N-(2-amino-2-methylpropyl)-N-{1-[3-fluoro-5-(trifluoromethyl)phenyl]cyclobutyl}carbamate;
y. methyl N-{1-[2-fluoro-5-(trifluoromethyl)phenyl]cyclobutyl}-N-{[(2S)-pyrrolidin-2-yl]methyl}carbamate;
z. methyl N-[1-(5-chloro-2-fluorophenyl)cyclobutyl]-N-{[(2S)-pyrrolidin-2-yl]methyl}carbamate;
aa. methyl N-(2-amino-2-methylpropyl)-N-[1-(5-chloro-2-fluorophenyl)cyclobutyl]carbamate;
bb. 1-(3-cyclopropyl-4-fluorophenyl)-N-{[(2R)-pyrrolidin-2-yl]methyl}cyclobutan-1-amine; and
cc. 1-(3-cyclopropyl-4-fluorophenyl)-N-{[(2S)-pyrrolidin-2-yl]methyl}cyclobutan-1-amine.

116. A pharmaceutical composition comprising the compound according to any one of the preceding items.

117. The compound or pharmaceutical composition according to any one of the preceding items for use in medicine.

118. The compound according to any one of items 1 to 115 or pharmaceutical composition according to item 116 for use in the treatment of inflammatory bowel disease (IBD).

119. The compound or pharmaceutical composition for use according to item 118, wherein the IBD is colitis.

120. The compound or pharmaceutical composition for use according to item 118, wherein the IBD is ulcerative colitis.

121. The compound or pharmaceutical composition for use according to item 118, wherein the IBD is Crohn's disease.

122. The compound according to any one of items 1 to 115 or pharmaceutical composition according to item 116 for use in the treatment of hereditary xerocytosis.

123. The compound according to any one of items 1 to 115 or pharmaceutical composition according to item 116 for use in the treatment of acute respiratory distress syndrome (ARDS).

124. A method for treatment of IBD, hereditary xerocytosis or ARDS comprising administration of the compound defined in any one of items 1 to 115 or a composition according to item 116 to a subject in need thereof.

125. Use of the compound defined in any one of items 1 to 115 or the composition according to item 116 in the manufacture of a medicament for treatment of IBD, hereditary xerocytosis or ARDS.

### EXAMPLES

### Example 1

### N-{[(2S)-pyrrolidin-2-yl]methyl}-1-[3-(trifluoromethyl)phenyl]cyclobutan-1-amine [1]

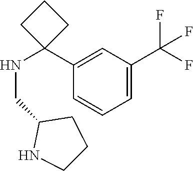 [SMILES: FC(F)(F)c1cccc(C2(NC[C@@H]3CCCN3)CCC2)c1]

### Step 1

To a solution of 3-(trifluoromethyl)phenylacetonitrile (10 g, 54 mmol) in THF (80 mL) at 0° C. was slowly added sodium hydride (60%, 2.6 g, 65 mmol). The reaction was stirred for 15 min, then 1,3-dibromopropane was added and the reaction stirred at RT for 12 h. The reaction mass was diluted with water and extracted with ethyl acetate. The organic layer was washed with water and brine solution, dried over sodium sulfate and concentrated to get crude product which was purified by using combi flash column by eluting with 10% ethyl acetate in hexane to afford product [1.1] as a sticky liquid (7.5 g, 60%).

### Step 2

To a solution of [1.1] (7.5 g, 33 mmol) in methanol (80 mL) at 0° C. was added tetrabutylammonium bromide (0.54 g 1.7 mmol), aqueous sodium hydroxide solution (3M, 3 mL) and aqueous hydrogen peroxide solution (30%, 11.3 g, 100 mmol) and the reaction stirred at RT for 12h. The reaction mass was diluted with water and extracted with ethyl acetate. The organic layer was washed with water and brine solution, dried over sodium sulfate and concentrated to get crude product which was purified by using combi flash column by eluting with 35% ethyl acetate in hexane to afford [1.2] as colourless liquid (5 g, 63%). LCMS: m/z: 244.3 (M+H).

### Step 3

To a solution of [1.2] (5 g, 20.5 mmol) in n-butanol (25 mL) at 0° C. was slowly added aq. NaOH solution (3M, 20.5 mL) and aq. sodium hypochlorite solution (6.7%, 13 mL) and the reaction stirred at RT for 12h. The reaction mass was diluted with water and extracted with ethyl acetate. The organic layer was washed with water and brine solution, dried over sodium sulfate and concentrated to afford crude product which was purified by using combi flash column by eluting with 15% ethyl acetate in hexane to afford [1.3] as a colourless liquid (1.1 g, 25%).

### Step 4

To a solution of [1.3] (0.2 g, 0.92 mmol) in methanol (10 mL) was added tert-butyl(S)-2-formylpyrrolidine-1-carboxylate (0.19 g, 0.92 mmol) and the reaction stirred at RT for 15 mins, then cooled to 0° C. Sodium cyanoborohydride (0.12 g, 1.86 mmol) was added portion-wise and the reaction stirred at RT for 12h. The reaction mass was diluted with water and extracted with ethyl acetate. The organic layer was washed with water and brine solution, dried over sodium sulfate and concentrated to get crude product [1.4](85% purity, 200 mg, ˜50%) which was used without out further purification. LCMS: m/z: 399.5 (M+H).

### Step 5

Methanolic HCl (3M, 0.5 mL) was added to [1.4] (0.2 g, 0.05 mmol) at 0° C. and the reaction stirred at RT for 2h. The reaction mass was concentrated completely and triturated with ether and pentane to give the product [1] as a hydrochloride salt (14 mg, 76%). LCMS: m/z: 299.1 (M+H).

¹H NMR (400 MHz, DMSO-d₆) δ ppm 1.53-1.66 (m, 1H), 1.65-1.91 (m, 3H), 2.06-2.22 (m, 1H), 2.73-3.05 (m, 6H), 3.12-3.24 (m, 3H), 3.76-3.92 (m, 1H), 7.62-7.77 (m, 1H), 7.80-7.89 (m, 1H), 7.91-8.14 (m, 2H), 9.1 (bs, 1H), 9.3 (bs, 1H), 10.2 (bs, 1H), 10.5 (bs, 1H).

### Example 2

### N-{[(2R)-pyrrolidin-2-yl]methyl}-1-[3-(trifluoromethyl)phenyl]cyclobutan-1-amine [2]

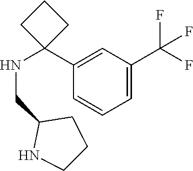 [SMILES: FC(F)(F)c1cccc(C2(NC[C@H]3CCCN3)CCC2)c1]

### Step 1

To a solution of [1.3] (0.25 g, 1.16 mmol) in DCM (10 mL) and isopropanol (6 mL) was added tert-butyl (R)-2-formylpyrrolidine-1-carboxylate (0.23 g, 1.16 mmol) and the reaction stirred for 15 min. Sodium triacetoxyborohydride (0.49 g, 2.32 mmol) was added in portions at 0° C. and the reaction stirred at RT for 2h. Water was added and the aqueous solution extracted twice with DCM. The combined organic layers were washed with water and brine solution, dried over sodium sulfate and concentrated to afford crude product [2.1] (0.3 g) which was used directly in the next step.

### Step 2

To a solution of [2.1] (0.1 g, 0.25 mmol) in DCM (10 mL) at 0° C. was added methanolic HCl (3M, 1 mL) and the reaction stirred for 3h. The reaction was concentrated under vacuum, triturated with hexane and the solid collected by filtration to afford product [2] as hydrochloride salt (55 mg, 73%).

¹H NMR (300 MHz, DMSO-d₆) δ ppm 1.52-1.92 (m, 4H), 1.99-2.21 (m, 2H), 2.55-2.71 (m, 2H), 2.84-3.03 (m, 3H), 3.35-3.55 (m, 3H), 3.76-3.95 (m, 1H), 7.59-7.81 (m, 2H), 7.88-8.09 (m, 2H), 8.85 (bs, 1H), 9.32 (bs, 1H), 9.45 (bs, 1H), 10.53 (bs, 1H).

### Example 3

### 2-methyl-N1-{1-[3-(trifluoromethyl)phenyl]cyclobutyl}propane-1,2-diamine [3]

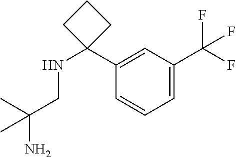 [SMILES: CC(C)(N)CNC1(c2cccc(C(F)(F)F)c2)CCC1]

### Step 1

To a solution of [6.1] (0.04 g, 0.10 mmol) in DCM (3 mL) at 0° C. was added methanolic HCl (3M, 0.5 mL) and the reaction stirred at RT for 3h. The reaction was concentrated under vacuum, triturated with pentane and the solid collected by filtration to afford product [3] as hydrochloride salt (12 mg, 40%).

¹H NMR (300 MHz, DMSO-d₆) δ ppm 1.028 (s, 6H), 1.56-1.80 (m, 1H), 1.98-2.24 (m, 1H), 2.53-2.83 (m, 4H) 2.85-3.18 (m, 2H) 7.63-7.89 (m, 2H) 7.94-8.07 (m, 1H) 8.01-8.19 (m, 1H), 8.68 (m, 3H), 10.25 (bs, 2H)

### Example 4

### methyl N-{[(2S)-pyrrolidin-2-yl]methyl}-N-{1-[3-(trifluoromethyl)phenyl]cyclobutyl}carbamate [4]

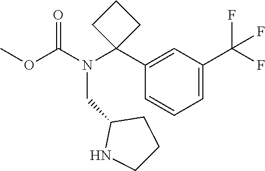 [SMILES: COC(=O)N(C[C@@H]1CCCN1)C1(c2cccc(C(F)(F)F)c2)CCC1]

### Step 1

To a solution of [1.3] (0.15 g, 70 mmol) in DCM (6 mL) and isopropanol (4 mL) at 0° C. was added tert-butyl (S)-2-formylpyrrolidine-1-carboxylate (0.14 g, 0.7 mmol) and the reaction stirred for 15 min. Sodium triacetoxyborohydride (0.30 g, 1.4 mmol) was added in portions and the reaction stirred at RT for 4h. Water was added and the aqueous solution extracted twice with DCM. The combined organic layers were washed with water and brine solution, dried over sodium sulfate and concentrated to afford crude product [4.1] (0.2 g, 72%) which was used directly in the next step.

### Step 2

To a solution of [4.1] (0.2 g, 0.5 mmol) in DCM (10 mL) at 0° C. was added N,N-diisopropylethylamine (0.17 mL, 0.13 g, 1.0 mmol) and methyl chloroformate (0.06 g, 0.6 mmol) and the reaction stirred at RT for 4h. Water was added and the aqueous solution extracted twice with DCM. The combined organic layers were washed with brine solution, dried over sodium sulphate, filtered and evaporated under high vacuum to get crude product [4.2] (0.2 g, 42%) which was used directly in the next step.

### Step 3

To a solution of [4.2] (0.15 g, 0.33 mmol) in DCM (5 mL) at 0° C. was added methanolic HCl (3M, 2 mL) and the reaction stirred at RT for 2h. The reaction was concentrated under vacuum, triturated with pentane and the solid collected by filtration to afford product [4] as hydrochloride salt (0.06 g, 46%).

¹H NMR (400 MHz, DMSO-d₆) δ ppm 1.47-1.68 (m, 2H), 1.76-2.09 (m, 4H), 2.57-2.73 (m, 3H), 3.02-3.18 (m, 1H) 3.19-3.32 (m, 2H) 3.40-3.61 (m, 4H) 3.59-3.73 (m, 1H) 7.55-7.73 (m, 2H) 7.75-7.83 (m, 1H) 7.82-7.90 (m, 1H) 8.53 (bs, 1H) 9.38 (bs, 1H).

### Example 5

### methyl N-{[(2R)-pyrrolidin-2-yl]methyl}-N-{1-[3-(trifluoromethyl)phenyl]cyclobutyl}carbamate [5]

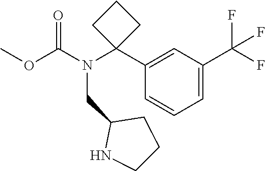 [SMILES: COC(=O)N(C[C@H]1CCCN1)C1(c2cccc(C(F)(F)F)c2)CCC1]

### Step 1

To a solution of [2.1] (0.025 g, 0.06 mmol) in DCM (5 mL) at 0° C. was added N,N-diisopropylethylamine (0.022 mL, 0.0.016 g, 0.13 mmol) and methyl chloroformate (0.007 g, 0.075 mmol) and the reaction stirred at RT for 4h. Water was added and the aqueous solution extracted twice with EtOAc. The combined organic layers were washed with brine solution, dried over sodium sulphate, filtered and evaporated under high vacuum to get crude product [5.1] (30 mg, quant.) which was used directly in the next step.

### Step 2

To a solution of [5.1] (29 mg, 0.06 mmol) in DCM (5 mL) at 0° C. was added methanolic HCl (3M, 0.5 mL) and the reaction stirred for 2h. The reaction was concentrated under vacuum, triturated with hexane and the solid collected by filtration to afford product [5] as hydrochloride salt (24 mg, 97%).

¹H NMR (300 MHz, DMSO-d₆) δ ppm 1.44-1.69 (m, 2H), 1.74-2.14 (m, 4H), 2.53-2.75 (m, 2H), 3.07-3.31 (m, 2H), 3.45-3.62 (m, 2H), 4.08-4.38 (m, 1H) 7.53-7.77 (m, 2H) 7.72-7.96 (m, 2H) 8.5 (bs, 1H), 9.30 (bs, 1H).

### Example 6

### methyl N-(2-amino-2-methylpropyl)-N-{1-[3-(trifluoromethyl)phenyl]cyclobutyl}carbamate [6]

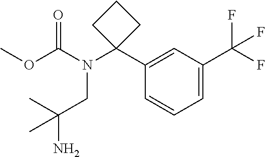 [SMILES: COC(=O)N(CC(C)(C)N)C1(c2cccc(C(F)(F)F)c2)CCC1]

### Step 1

To a solution of [1.3] (0.2 g, 0.92 mmol) in DCM (5 mL) and isopropanol (5 mL) at 0° C. was added tert-butyl 2-formylpropan-2-ylcarbamate (0.17 g, 0.92 mmol) and the reaction stirred for 2h. Sodium triacetoxyborohydride (0.39 g, 1.86 mmol) was added in portions at 0° C. and the reaction stirred at RT for 2h. Water was added and the aqueous solution extracted twice with DCM. The combined organic layers were washed with water and brine solution, dried over sodium sulfate and concentrated to afford crude product [6.1] (0.15 g) which was used directly in the next step.

### Step 2

To a solution of [6.1] (0.15 g, 0.39 mmol) in DCM (10 mL) at 0° C. was added N,N-diisopropylethylamine (0.1 mL, 0.14 g, 0.77 mmol) and methyl chloroformate (0.044 g, 0.47 mmol) and the reaction stirred at RT for 4h. Water was added and the aqueous solution extracted twice with EtOAc. The combined organic layers were washed with brine solution, dried over sodium sulphate, filtered and evaporated under high vacuum to get crude product which was purified by using combi flash column by eluting with 10% ethyl acetate in hexane to afford product [6] (50 mg, 29%).

¹H NMR (300 MHz, METHANOL-d₄) δ ppm 1.54 (s, 6H), 1.62-1.73 (m, 1H), 1.83-1.93 (m, 1H), 2.62-2.72 (m, 2H), 3.15-3.26 (m, 1H), 3.54 (s, 3H), 3.64-3.72 (m, 1 H), 7.52-7.66 (m, 1H), 7.64-7.73 (m, 1H), 7.70-7.85 (m, 2H).

### Example 7

### N1-[1-(3-chlorophenyl)cyclobutyl]-2-methylpropane-1,2-diamine [7]

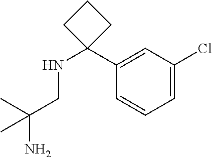 [SMILES: CC(C)(N)CNC1(c2cccc(Cl)c2)CCC1]

### Step 1

To a solution of 3-chlorobenzyl cyanide (10 g, 66 mmol) in THF (50 mL) at 0° C. was added sodium hydride (60%, 3.2 g, 79 mmol). After 15 min., 1,3-dibromopropane (16 g, 79 mmol) was added and the reaction stirred at RT for 12 h. The reaction was diluted with water and extracted with ethyl acetate. The combined organic layers were washed with water and brine solution, dried over sodium sulfate, filtered and concentrated under vacuum to afford crude product which was purified combi flash column eluting with 10% ethyl acetate in hexane to afford product [7.1] as a sticky liquid (7 g, 55%).

### Step 2

To a solution of [7.1] (7 g, 36.5 mmol) in methanol (50 mL) at 0° C. was added Tetrabutylammonium bromide (0.59 g, 1.8 mmol), sodium hydroxide solution (3M, 3 mL) and hydrogen peroxide solution (30%, 12.4 g) and the reaction stirred at RT for 12h. The reaction was diluted with water and extracted with ethyl acetate. The combined organic layers were washed with water and brine solution, dried over sodium sulfate, filtered and concentrated under vacuum to afford crude product [7.2] (6 g, 78%) which was used without further purification.

### Step 3

To a solution of [7.2] (1 g, 4.8 mmol) in n-butanol (5 mL) at 0° C. was slowly added aq. NaOH solution (3M, 4.8 mL) and aq. sodium hypochlorite solution (6.7%, 3 mL) and the reaction stirred at RT for 12h. The reaction mass was diluted with water and extracted with ethyl acetate. The organic layer was washed with water and brine solution, dried over sodium sulfate and concentrated to afford crude product [7.3] (0.6 g) which was used without further purification.

### Step 4

To a solution of [7.3] (0.2 g, 1.1 mmol) in DCM (5 mL) and isopropanol (5 mL) at RT was added tert-butyl 2-formylpropan-2-ylcarbamate (0.17 g, 0.92 mmol) and the reaction stirred for 2h. Sodium triacetoxyborohydride (0.46 g, 2.2 mmol) was added in portions at 0° C. and the reaction stirred at RT for 3h. Water was added and the aqueous solution extracted twice with DCM. The combined organic layers were washed with water and brine solution, dried over sodium sulfate and concentrated to afford crude product [7.4] (0.3 g) which was used directly in the next step.

### Step 5

To a stirred solution of [7.4] (0.3 g, 0.85 mmol) in DCM (10 mL) at 0° C. was added methanolic HCl (3M, 4 mL) and the reaction stirred for 2h. The reaction was concentrated under vacuum and purified by prep HPLC. The free base was dissolved in DCM and converted to the hydrochloride salt using methanolic HCl. The solvents were evaporated under vacuum, the solid residue triturated with hexane and the solid collected by filtration to afford product [7] as hydrochloride salt (36 mg, 13%).

¹H NMR (300 MHz, DMSO-d₆) δ ppm 1.3 (s, 6H), 1.52-1.75 (m, 1H), 1.93-2.18 (m, 1H), 2.50-2.85 (m, 4H), 2.80-3.05 (m, 2H), 7.33-7.65 (m, 2H), 7.57-7.73 (m, 1 H), 7.70-7.86 (m, 1H), 8.62 (m, 3H), 10.2 (bs, 2H)

### Example 8

### Methyl N-[1-(3-chlorophenyl)cyclobutyl]-N-{[(2S)-pyrrolidin-2-yl]methyl}carbamate [8]

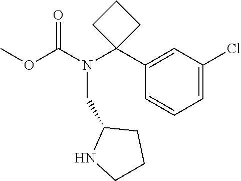 [SMILES: COC(=O)N(C[C@@H]1CCCN1)C1(c2cccc(Cl)c2)CCC1]

### Step 1

To a solution of [7.3] (0.15 g, 70 mmol) in DCM (6 mL) and isopropanol (4 mL) at 0° C. was added tert-butyl (S)-2-formylpyrrolidine-1-carboxylate (0.14 g, 0.7 mmol) and the reaction stirred for 15 min. Sodium triacetoxyborohydride (0.30 g, 1.4 mmol) was added in portions and the reaction stirred at RT for 4h. Water was added and the aqueous solution extracted twice with DCM. The combined organic layers were washed with water and brine solution, dried over sodium sulfate and concentrated to afford crude product [8.1] (0.18 g, 71%) which was used directly in the next step.

### Step 2

To a solution of [8.1] (0.18 g, 0.49 mmol) in DCM (20 mL) at 0° C. was added N,N-diisopropylethylamine (0.16 mL, 0.12 g, 0.9 mmol) and methyl chloroformate (0.05 g, 0.54 mmol) and the reaction stirred at RT for 4h. Water was added and the aqueous solution extracted twice with DCM. The combined organic layers were washed with brine solution, dried over sodium sulphate, filtered and evaporated under high vacuum to get crude product which was purified by prep-HPLC to afford [8.2] (0.08 g, 42%)

### Step 3

To a solution of [8.2] (0.1 g, 0.24 mmol) in DCM (5 mL) at 0° C. was added methanolic HCl (3M, 1 mL) and the reaction stirred at RT for 2h. The reaction was concentrated under vacuum, triturated with pentane and the solid collected by filtration to afford product [8] as hydrochloride salt (0.032 g, 42%).

¹H NMR (300 MHz, DMSO-d₆) δ ppm 1.44-1.66 (m, 2H), 1.72-1.96 (m, 3H), 1.95-2.09 (m, 1H), 2.53-2.69 (m, 3H), 2.97-3.19 (m, 2H), 3.19-3.29 (m, 2H), 3.35-3.48 (m, 1H) 3.50-3.58 (m, 3H), 3.59-3.74 (m, 1H), 7.19-7.61 (m, 4H), 8.31 (bs, 1H), 9.14 (bs, 1H).

### Example 9

### Methyl N-[1-(3-chlorophenyl)cyclobutyl]-N-{[(2R)-pyrrolidin-2-yl]methyl}carbamate [9]

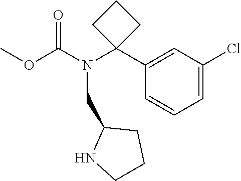 [SMILES: COC(=O)N(C[C@H]1CCCN1)C1(c2cccc(Cl)c2)CCC1]

### Step 1

To a solution of [7.3] (0.15 g, 70 mmol) in DCM (6 mL) and isopropanol (4 mL) at 0° C. was added tert-butyl (R)-2-formylpyrrolidine-1-carboxylate (0.16 g, 0.83 mmol) and the reaction stirred for 15 min. Sodium triacetoxyborohydride (0.35 g, 1.7 mmol) was added in portions and the reaction stirred at RT for 4h. Water was added and the aqueous solution extracted twice with DCM. The combined organic layers were washed with water and brine solution, dried over sodium sulfate and concentrated to afford crude product [9.1] (0.3 g, crude) which was used directly in the next step.

### Step 2

To a solution of [9.1] (0.15 g, 0.41 mmol) in DCM (10 mL) at 0° C. was added N,N-diisopropylethylamine (0.14 mL, 0.11 g, 0.8 mmol) and methyl chloroformate (0.047 g, 0.49 mmol) and the reaction stirred at RT for 4h. Water was added and the aqueous solution extracted twice with DCM. The combined organic layers were washed with brine solution, dried over sodium sulphate, filtered and evaporated under high vacuum to afford crude product [9.2] (0.15 g, crude) which was used directly in the next step.

### Step 3

To a solution of [9.2] (0.12 g, 0.28 mmol) in DCM (5 mL) at 0° C. was added methanolic HCl (3M, 2 mL) and the reaction stirred at RT for 2h. The reaction was concentrated under vacuum and the residue purified by prep-HPLC. The pure product was treated with methanolic HCl to afford a solid which was triturated with ether and pentane and the solid collected by filtration to afford product [9] as hydrochloride salt (0.037 g, 36%).

¹H NMR (400 MHz, DMSO-d₆) δ ppm 1.48-1.66 (m, 2H), 1.64-2.09 (m, 4H), 2.53-2.77 (m, 3H), 2.98-3.31 (m, 2H), 3.41-3.52 (m, 3H), 3.55 (s, 3H), 3.56-3.77 (m, 1H) 7.21-7.59 (m, 4H) 8.40 (bs, 1H) 9.20 (bs, 1H)

### Example 10

### N-{[(2S)-pyrrolidin-2-yl]methyl}-1-[3-(trifluoromethoxy)phenyl]cyclobutan-1-amine [10]

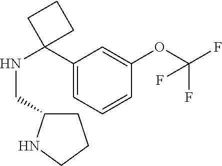 [SMILES: FC(F)(F)Oc1cccc(C2(NC[C@@H]3CCCN3)CCC2)c1]

### Step 1

To a solution of potassium hydroxide (7 g, 124 mmol) in water (4 mL) and toluene (40 mL) at 50° C. was added 1,3-dibromopropane (5.5 g, 27 mmol) and tetrabutylammonium bromide (0.40 g, 1.24 mmol). A solution of 3-(trifluoromethoxy)phenylacetonitrile (5 g, 25 mmol) in toluene (4 mL) was slowly added to the reaction and the temperature raised to 90° C. and stirred for 1h. After cooling, the reaction mixture was extracted twice with DCM and the combined organic layers were washed with brine solution, dried over sodium sulphate, evaporated under high vacuum to get crude compound which was purified by using combi flash column by eluting with 5% ethyl acetate in hexane to afford [10.1] (4.5 g, 75%).

### Step 2

To a solution of [10.1] (4.5 g, 17.5 mmol) in acetic acid (18 mL) was added dropwise conc. sulfuric acid (9 mL) at 0° C. The temperature was raised to 90° C. and the reaction stirred for 16h. Ice cold water was added to the reaction mixture and the aqueous solution extracted twice with DCM. The combined organic layers were washed with sodium bicarbonate solution and brine solution, dried over sodium sulphate, evaporated under high vacuum to afford product [10.2] (3.3 g, crude) which was used without further purification.

### Step 3

To a stirred solution of [10.2] (2.7 g, 9.8 mmol) in t-butanol (27 mL) at 50° C. was added lead tetraacetate (4.8 g, 11 mmol) in portions and the reaction stirred at 80° C. for 2h. The reaction mixture was diluted with EtOAc and filtered through celite. The filtrate was evaporated under high vacuum to get crude compound which was purified by using combi flash column by eluting with 15% ethyl acetate in hexane to afford the product [10.3] as a white solid (2.1 g, 65%).

### Step 4

To a solution of [10.3] (2.1 g, 6 mmol) in methanol (20 mL) was added methanolic HCl (3M, 4 mL) and the reaction stirred at RT for 4h. The reaction was concentrated under vacuum, triturated with hexane and the solid collected by filtration to afford product [10.4] as hydrochloride salt (1.5 g, 93%).

### Step 5

To a solution of [10.4] (0.25 g, 0.93 mmol) in DCM (3 mL) and isopropanol (2 mL) at 0° C. was added tert-butyl (S)-2-formylpyrrolidine-1-carboxylate (0.19 g, 0.93 mmol) and the reaction stirred for 15 min. Sodium triacetoxyborohydride (0.30 g, 1.4 mmol) was added in portions and the reaction stirred at RT for 2h. Water was added and the aqueous solution extracted twice with DCM. The combined organic layers were washed with water and brine solution, dried over sodium sulfate and concentrated to afford crude product which was purified by using combi flash column by eluting with 15% ethyl acetate in hexane to afford [10.5] (0.25 g, 65%).

### Step 6

To a solution of [10.5] (0.15 g, 0.36 mmol) in DCM (2 mL) was added methanolic HCl (3M, 0.24 mL) and the reaction stirred at RT for 3h. The reaction was concentrated under vacuum, the solid residue triturated with hexane and the solid collected by filtration to afford product [10] as hydrochloride salt (0.074 g, 58%).

¹H NMR (400 MHz, DMSO-d₆) δ ppm 1.51-1.64 (m, 1H), 1.66-1.73 (m, 1H), 1.75-1.96 (m, 3H), 2.05-2.19 (m, 2H), 2.54-2.72 (m, 2H), 2.78-3.07 (m, 3H), 3.14-3.25 (m, 2H), 3.80-3.92 (m, 1H), 7.42-7.54 (m, 1H), 7.57-7.78 (m, 3H), 9.21 (bs, 1H), 9.37 (bs, 1H), 10.28 (bs, 1H) 10.52 (bs, 1H).

### Example 11

### N-{[(2R)-pyrrolidin-2-yl]methyl}-1-[3-(trifluoromethoxy)phenyl]cyclobutan-1-amine [11]

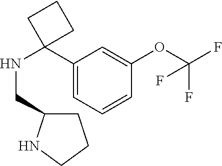 [SMILES: FC(F)(F)Oc1cccc(C2(NC[C@H]3CCCN3)CCC2)c1]

### Step 1

To a solution of [10.4] (0.25 g, 0.93 mmol) in DCM (3 mL) and isopropanol (2 mL) at 0° C. was added tert-butyl (R)-2-formylpyrrolidine-1-carboxylate (0.19 g, 0.93 mmol) and the reaction stirred for 15 min. Sodium triacetoxyborohydride (0.30 g, 1.4 mmol) was added in portions and the reaction stirred at RT for 2h. Water was added and the aqueous solution extracted twice with DCM. The combined organic layers were washed with water and brine solution, dried over sodium sulfate and concentrated to afford crude product which was purified by using combi flash column by eluting with 15% ethyl acetate in hexane to afford [11.1] (0.2 g, 52%).

### Step 2

To a solution of [11.1] (0.07 g, 0.17 mmol) in DCM (1 mL) was added methanolic HCl (3M, 0.11 mL) and the reaction stirred at RT for 3h. The reaction was concentrated under vacuum, the solid residue triturated with hexane and the solid collected by filtration to afford product [11] as hydrochloride salt (0.042 g, 71%).

¹H NMR (400 MHz, DMSO-d₆) δ ppm 1.18-1.34 (m, 1H) 1.56-1.95 (m, 4H) 2.03-2.22 (m, 2H) 2.57-2.74 (m, 2H) 2.77-3.00 (m, 3H) 3.08-3.21 (m, 2H) 3.76-3.90 (m, 1H) 7.40-7.56 (m, 1H) 7.53-7.81 (m, 3H) 9.15 (bs, 1H) 9.29 (bs, 1H) 10.23 (bs, 1H), 10.50 (bs, 1H).

### Example 12

### Methyl N-{[(2S)-pyrrolidin-2-yl]methyl}-N-{1-[3-(trifluoromethoxy)phenyl]cyclobutyl}carbamate [12]

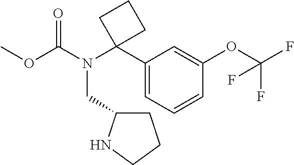 [SMILES: COC(=O)N(C[C@@H]1CCCN1)C1(c2cccc(OC(F)(F)F)c2)CCC1]

### Step 1

To a solution of [10.5] (0.1 g, 0.24 mmol) in DCM (2 mL) at 0° C. was added N,N-diisopropylethylamine (0.08 mL, 0.06 g, 0.48 mmol) and methyl chloroformate (0.045 g, 0.48 mmol) and the reaction stirred at RT for 2h. Water was added and the aqueous solution extracted twice with DCM. The combined organic layers were washed with brine solution, dried over sodium sulphate, filtered and evaporated under high vacuum to get crude product which was purified by prep-TLC to afford the product [12.1] (0.08 g, 73%).

### Step 2

To a solution of [12.1] (0.08 g, 0.17 mmol) in DCM (1 mL) was added methanolic HCl (3M, 0.11 mL) and the reaction stirred at RT for 3h. The reaction was concentrated under vacuum, the solid residue triturated with hexane and the solid collected by filtration to afford product [12] as hydrochloride salt (0.059 g, 85%).

¹H NMR (400 MHz, DMSO-d₆) δ ppm 1.15-1.34 (m, 1H), 1.49-1.70 (m, 2H), 1.73-2.11 (m, 4H), 2.55-2.74 (m, 2H), 2.97-3.24 (m, 2H), 3.20-3.37 (m, 1H), 3.40-3.47 (m, 1H), 3.54 (s, 3H), 3.61-3.80 (m, 2H), 7.27-7.37 (m, 1H), 7.37-7.66 (m, 3H), 8.51 (bs, 1H), 9.35 (bs, 1H).

### Example 13

### Methyl N-{[(2R)-pyrrolidin-2-yl]methyl}-N-{1-[3-(trifluoromethoxy)phenyl]cyclobutyl}carbamate [13]

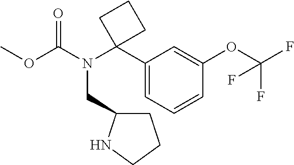 [SMILES: COC(=O)N(C[C@H]1CCCN1)C1(c2cccc(OC(F)(F)F)c2)CCC1]

### Step 1

To a solution of [11.1] (0.13 g, 0.31 mmol) in DCM (2 mL) at 0° C. was added N,N-diisopropylethylamine (0.11 mL, 0.08 g, 0.63 mmol) and methyl chloroformate (0.030 g, 0.31 mmol) and the reaction stirred at RT for 2h. Water was added and the aqueous solution extracted twice with DCM. The combined organic layers were washed with brine solution, dried over sodium sulphate, filtered and evaporated under high vacuum to get crude product which was purified by prep-TLC to afford the product [13.1] (0.12 g, 81%).

### Step 2

To a solution of [13.1] (0.12 g, 0.25 mmol) in DCM (2 mL) was added methanolic HCl (3M, 0.17 mL) and the reaction stirred at RT for 3h. The reaction was concentrated under vacuum, the solid residue triturated with hexane and the solid collected by filtration to afford product [13] as hydrochloride salt (0.071 g, 68%).

¹H NMR (400 MHz, DMSO-d₆) δ ppm 1.13-1.38 (m, 2H), 1.45-1.72 (m, 2H), 1.76-1.92 (m, 2H), 1.96-2.06 (m, 1H), 2.53-2.72 (m, 2H), 2.96-3.15 (m, 1H), 3.21-3.31 (m, 1H), 3.35-3.43 (m, 1H), 3.55-3.61 (m, 3H), 3.61-3.76 (m, 1H), 7.22-7.33 (m, 1H), 7.41-7.73 (m, 3H), 8.52 (bs, 1H), 9.35 (bs, 1H).

### Example 14

### Methyl N-(2-amino-2-methylpropyl)-N-{1-[3-(trifluoromethoxy)phenyl]cyclobutyl}carbamate [14]

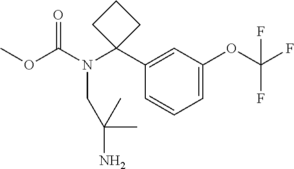 [SMILES: COC(=O)N(CC(C)(C)N)C1(c2cccc(OC(F)(F)F)c2)CCC1]

### Step 1

To a solution of [10.4] (0.25 g, 0.93 mmol) in DCM (3 mL) and isopropanol (2 mL) at 0° C. was added tert-butyl 2-formylpropan-2-ylcarbamate (0.17 g, 0.93 mmol) and the reaction stirred for 15 min. Sodium triacetoxyborohydride (0.30 g, 1.4 mmol) was added in portions at 0° C. and the reaction stirred at RT for 2h. Water was added and the aqueous solution extracted twice with DCM. The combined organic layers were washed with water and brine solution, dried over sodium sulfate and concentrated to afford crude product which was purified by prep-TLC to afford the product [14.1] (0.12 g, 32%).

### Step 2

To a solution of [14.1] (0.12 g, 0.30 mmol) in DCM (2 mL) at 0° C. was added N,N-diisopropylethylamine (0.10 mL, 0.08 g, 0.6 mmol) and methyl chloroformate (0.05 g, 0.6 mmol) and the reaction stirred at RT for 24h. Water was added and the aqueous solution extracted twice with DCM. The combined organic layers were washed with brine solution, dried over sodium sulphate, filtered and evaporated under high vacuum to get crude product which was purified by prep-HPLC to afford the product [14.2](0.045 g, 33%).

### Step 3

To a solution of [14.2] (0.045 g, 0.10 mmol) in DCM (2 mL) at 0° C. was added methanolic HCl (3M, 0.07 mL) and the reaction stirred at RT for 2h. The reaction was concentrated under vacuum, the solid residue triturated with hexane and the solid collected by filtration to afford product [14] as hydrochloride salt (0.011 g, 28%).

¹H NMR (400 MHz, DMSO-d₆) δ ppm 1.08-1.19 (m, 2H), 1.28 (s, 6H), 1.50-1.63 (m, 1H), 1.73-1.85 (m, 1H), 2.54-2.66 (m, 2H), 3.09-3.20 (m, 1H), 3.53 (s, 3H), 3.54-3.63 (m, 1H), 7.26-7.35 (m, 1H), 7.34-7.46 (m, 1H), 7.46-7.61 (m, 2H), 7.66-7.86 (m, 3H).

### Example 15

### 1-[4-fluoro-3-(trifluoromethyl)phenyl]-N-{[(2S)-pyrrolidin-2-yl]methyl}cyclobutan-1-amine [15]

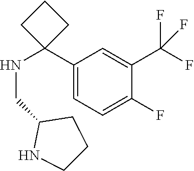 [SMILES: Fc1ccc(C2(NC[C@@H]3CCCN3)CCC2)cc1C(F)(F)F]

### Step 1

To a solution of 4-fluoro-3-(trifluoromethyl)-phenylacetic acid (10 g, 45 mmol) in ethanol (100 mL) was added Sulphuric acid (98%, 0.49 mL) and the reaction mixture was stirred at 80° C. for 18h. The reaction mixture was concentrated under vacuum and the residue was diluted with ethyl acetate. The organic layer was washed with 10% sodium bicarbonate solution, followed by water and brine. The organic layer was dried over sodium sulfate, filtered and concentrated to afford [15.1] as a yellowish gum (11 g, 98%, MH+=251.1).

### Step 2

To a solution of [15.1] (2 g, 8 mmol) in DMF (10 mL) was added sodium hydride (60%, 0.67 g) at 0° C. under nitrogen. After 30 min, 1,3-dibromopropane (1.94 g, 9.6 mmol) was added at 0° C. and the reaction stirred at RT for 1h. The reaction mixture was diluted with dichloromethane, and washed with water and brine solution. The organic layer was dried over sodium sulphate, filtered and concentrated under reduced pressure to afford crude product as a pale brown liquid, which was purified by column chromatography (ethyl acetate/hexane) to afford [15.2] as a yellow liquid (0.5 g, 22%).

### Step 3

To a solution of [15.2] (1 g, 3.45 mmol) in THF (2 mL), Ethanol (2 mL) and Water (2 mL) was added lithium hydroxide (0.17 g, 6.9 mmol). The reaction was stirred at RT under nitrogen for 18h and diluted with water. The aqueous layer was washed with ethyl acetate (2×10 mL), then neutralised with 1.5 N HCl (aq) and extracted with ethyl acetate (2*20 mL). The combined organic layers were washed with brine and dried over anhydrous Na2SO4 to obtain [15.3] as a white solid (0.6 g, 67%).

### Step 4

To a solution [15.3] (1.2 g, 4.6 mmol) in DCM (10 mL) was added diphenylphosphoryl azide (1.5 g, 5.5 mmol) and triethylamine (1.9 mL, 13.8 mmol) and the reaction mixture was stirred at room temperature for 13h. Excess solvent was removed under vacuum and the residue dissolved in tert-butanol (10 mL). Molecular sieves were added and the reaction heated at 70° C. for 12h. After completion of reaction, the molecular sieves were removed by filtration and the filtrate was concentrated under vacuum. The residue was purified by column chromatography over neutral silica (60-120 mesh, 20:80 ethyl acetate: pet ether) to yield [15.4] as a white solid (0.5 g, 33%).

### Step 5

To a solution of [15.4] (0.7 g, 2.1 mmol) in dichloromethane (3 mL) at 0° C. was added HCl gas in Dioxane (3 mL) and the reaction was stirred at RT under nitrogen for 18h.

The reaction was concentrated under vacuum and the residue triturated with diethyl ether and the solid collected by filtration to obtain the hydrochloride salt [15.5] as a white solid (0.3 g, 54%).

### Step 6

To a solution of [15.5] (0.3 g, 1.3 mmol) in methanol (5 mL) was added tert-butyl (S)-2-formylpyrrolidine-1-carboxylate (0.26 g, 1.3 mmol) and the reaction mixture was stirred at rt for 18h. Sodium cyanoborohydride (0.16 g, 2.6 mmol) was to the reaction mixture and the reaction stirred at rt for 18h. The reaction mixture was concentrated under vacuum. The residue was redissolved in dichloromethane and washed with water (20 mL), followed by brine. The organic layer was concentrated under vacuum to obtain crude product which was purified by column using Reveleris Grace instrument to obtain [15.6] as a yellow colour gum (0.4 g, 74%).

### Step 7

To a solution of [15.6] (0.08 g, 0.19 mmol) in dichloromethane (3 mL) at 0° C. was added HCl gas in Dioxane (3 mL). After 1h, the reaction mixture was concentrated under reduced pressure and the residue was freeze-dried to afford the hydrochloride salt [15] as a white solid (0.03 g, 46%).

¹H NMR (400 MHz, DMSO-d₆) δ ppm 1.58-1.93 (m, 4H), 2.08-2.13 (m, 2H), 2.50-2.75 (m, 2H), 2.75-3.10 (m, 4H), 3.18 (br s, 2H), 3.80-3.95 (br s, 1H), 7.60-7.73 (m, 1H), 7.95-8.10 (m, 2H), 9.20-9.39 (m, 2H), 10.30-10.62 (m, 2H).

### Example 16

### 1-[4-fluoro-3-(trifluoromethyl) phenyl]-N-{[(2R)-pyrrolidin-2-yl]methyl}cyclobutan-1-amine [16]

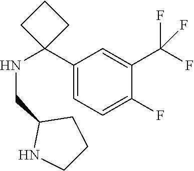 [SMILES: Fc1ccc(C2(NC[C@H]3CCCN3)CCC2)cc1C(F)(F)F]

### Step 1

To a solution of potassium hydroxide (13.8 g, 246 mmol) in water (80 mL) and toluene (4 mL) at 50° C. was added 1,3-dibromopropane (10.9 g, 54 mmol) and tetrabutylammonium bromide (0.8 g, 2.5 mmol). A solution of 4-fluoro-3-(trifluoromethyl)phenylacetonitrile (10 g, 49 mmol) in toluene (4 mL) was slowly added to the reaction and the temperature raised to 90° C. and stirred for 2h. After cooling, the reaction mixture was extracted twice with DCM and the combined organic layers were washed with brine solution, dried over sodium sulphate, evaporated under high vacuum to get crude compound which was purified by using combi flash column by eluting with 5% ethyl acetate in hexane to afford [16.1] (4 g, 33%).

### Step 2

To a solution of [16.1] (4 g, 16.4 mmol) in acetic acid (16 mL) was added dropwise conc. sulfuric acid (4 mL) at 0° C. The temperature was raised to 90° C. and the reaction stirred for 16h. Ice cold water was added to the reaction mixture and the aqueous solution extracted twice with DCM. The combined organic layers were washed with sodium bicarbonate solution and brine solution, dried over sodium sulphate, evaporated under high vacuum to afford product [16.2] (3.6 g, 86%) which was used without further purification.

### Step 3

To a stirred solution of [16.2] (3.6 g, 13.8 mmol) in t-butanol (30.6 mL) at 50° C. was added lead tetraacetate (6.7 g, 15 mmol) and the reaction stirred at 80° C. for 2h. The reaction mixture was diluted with EtOAc and filtered through celite. The filtrate was evaporated under high vacuum to get crude compound which was purified by using combi flash column by eluting with 5% ethyl acetate in hexane to afford the product [16.3] as a white solid (2.75 g, 60%).

### Step 4

To a solution of [16.3] (2.75 g, 8.25 mmol) in DCM (15 mL) was added methanolic HCl (3M, 15 mL) and the reaction stirred for 3h. The reaction was concentrated under vacuum, triturated with hexane and the solid collected by filtration to afford product [16.4] as hydrochloride salt (1.8 g, 81%).

### Step 5

To a solution of [16.4] (0.15 g, 0.64 mmol) in DCM (1.5 mL) and isopropanol (1 mL) at 0° C. was added tert-butyl (R)-2-formylpyrrolidine-1-carboxylate (0.13 g, 0.64 mmol) and the reaction stirred for 15 min. Sodium triacetoxyborohydride (0.20 g, 0.96 mmol) was added in portions and the reaction stirred at RT for 2h. Water was added and the aqueous solution extracted twice with DCM. The combined organic layers were washed with water and brine solution, dried over sodium sulfate and concentrated to afford crude product which was purified by using combi flash column by eluting with 15% ethyl acetate in hexane to afford [16.5] (0.18 g, 69%).

### Step 6

To a stirred solution of [16.5] (0.05 g, 0.12 mmol) in DCM (1 mL) at 0° C. was added methanolic HCl (3M, 0.04 mL) and the reaction stirred for 3h. The reaction was concentrated under vacuum, triturated with hexane and the solid collected by filtration to afford product [16] as hydrochloride salt (22 mg, 57%).

¹H NMR (400 MHz, DMSO-d₆) δ ppm 1.52-1.96 (m, 4H), 2.02-2.22 (m, 2H), 2.59-3.21 (m, 7H), 3.42-3.69 (m, 1H), 3.78-4.03 (m, 1H), 7.58-7.77 (m, 1H), 7.87-8.23 (m, 2H), 9.12-9.30 (bs, 1H), 9.32-9.56 (bs, 2H), 10.28-10.42 (bs, 1H), 10.44 10.70 (bs, 1H).

### Example 17

### N1-{1-[4-fluoro-3-(trifluoromethyl)phenyl]cyclobutyl}-2-methylpropane-1,2-diamine [17]

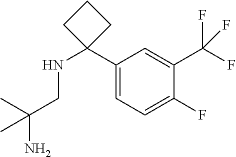 [SMILES: CC(C)(N)CNC1(c2ccc(F)c(C(F)(F)F)c2)CCC1]

### Step 1

To a solution of [16.4] (0.25 g, 1.1 mmol) in DCM (3 mL) and isopropanol (2 mL) at 0° C. was added tert-butyl 2-formylpropan-2-ylcarbamate (0.2 g, 1.1 mmol) and the reaction stirred for 15 min. Sodium triacetoxyborohydride (0.34 g, 1.61 mmol) was added in portions and the reaction stirred at RT for 2h. Water was added and the aqueous solution extracted twice with DCM. The combined organic layers were washed with water and brine solution, dried over sodium sulfate and concentrated to afford crude product which was purified by using prep LCMS to afford [17.1] (0.16 g, 37%).

### Step 2

To a stirred solution of [17.1] (0.05 g, 0.12 mmol) in DCM (1 mL) at 0° C. was added methanolic HCl (3M, 0.04 mL) and the reaction stirred for 3h. The reaction was concentrated under vacuum, triturated with hexane and the solid collected by filtration to afford product [17] as hydrochloride salt (19 mg, 51%).

¹H NMR (400 MHz, METHANOL-d₄) δ ppm 1.40 (s, 6H), 1.80-1.92 (m, 1H), 2.21-2.32 (m, 1H), 2.78-2.88 (m, 4H), 2.90-3.05 (m, 2H), 7.51-7.55 (m, 1H), 8.01-8.04 (m, 2H).

### Example 18

### methyl N-{1-[4-fluoro-3-(trifluoromethyl)phenyl]cyclobutyl}-N-{[(2S)-pyrrolidin-2-yl]methyl}carbamate [18]

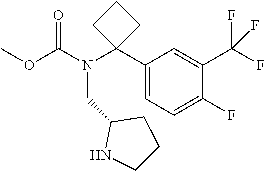 [SMILES: COC(=O)N(C[C@@H]1CCCN1)C1(c2ccc(F)c(C(F)(F)F)c2)CCC1]

### Step 1

To a solution of [15.6] (0.24 g, 0.58 mmol) in acetonitrile (5 mL) was added potassium carbonate (0.16 g, 1.15 mmol) and methyl chloroformate (0.06 g, 0.63 mmol) and the reaction mixture was stirred at RT for 16h. The reaction mixture was concentrated under vacuum and the residue was diluted with ethyl acetate washed with water and brine solution. The organic layer was dried over sodium sulphate, filtered and concentrated under reduced pressure to afford crude product which was purified by flash column chromatography (ethyl acetate/pet ether) to afford [18.1] as a colourless gum (0.24 g, 89%).

### Step 2

To a solution of [18.1] (0.23 g, 0.48 mmol) in DCM (3 mL) at 0° C. was added HCl gas in diethyl ether (3 mL) and the reaction was stirred at RT under nitrogen. After 1h, the reaction mixture was concentrated under reduced pressure and the residue was freeze-dried to afford the hydrochloride salt [18] as a white solid (0.13 g, 65%).

¹H NMR (400 MHz, DMSO-d₆) δ ppm 1.45-1.65 (m, 2H), 1.75-2.00 (m, 3H), 2.02-2.12 (m, 1H), 2.50-2.80 (obs m, 4H), 3.05-3.20 (m, 2H), 3.40-3.70 (m, 3H), 3.56 (s, 3H), 7.50-7.56 (m, 1H), 7.81 (br s, 1H), 7.94 (br s, 1H), 8.25-8.45 (br s, 1H), 9.10-9.30 (br s, 1H).

### Example 19

### methyl N-{1-[4-fluoro-3-(trifluoromethyl)phenyl]cyclobutyl}-N-{[(2R)-pyrrolidin-2-yl]methyl}carbamate [19]

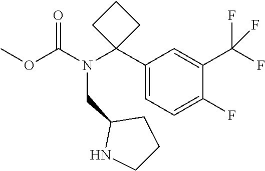 [SMILES: COC(=O)N(C[C@H]1CCCN1)C1(c2ccc(F)c(C(F)(F)F)c2)CCC1]

### Step 1

To a solution of [16.5] (0.13 g, 0.31 mmol) in DCM (2 mL) at 0° C. was added N,N-diisopropylethylamine (0.11 mL, 0.08 g, 0.62 mmol) and methyl chloroformate (0.06 g, 0.62 mmol) and the reaction stirred at RT for 12h. Water was added and the aqueous solution extracted twice with DCM. The combined organic layers were washed with brine solution, dried over sodium sulphate, filtered and evaporated under high vacuum to get crude product [19.1] (100 mg, 68%) which was used directly in the next step.

### Step 2

To a solution of [19.1] (0.100 g, 0.21 mmol) in DCM (1 mL) was added methanolic HCl (3M, 0.07 mL) and the reaction stirred at RT for 3h. The reaction was concentrated under vacuum, triturated with hexane and the solid collected by filtration to afford product [19] as hydrochloride salt (40 mg, 45%).

¹H NMR (400 MHz, DMSO-d₆) δ ppm 1.15-1.39 (m, 1H), 1.45-1.71 (m, 2H), 1.71-2.12 (m, 4H), 2.53-2.74 (m, 3H), 3.03-3.34 (m, 2H), 3.41-3.72 (m, 5H), 3.67-3.99 (m, 2H), 7.41-7.61 (m, 1H), 7.73-7.96 (m, 2H), 8.70 (bs, 1H) 9.53 (bs, 1H).

### Example 20

### methyl N-(2-amino-2-methylpropyl)-N-{1-[4-fluoro-3-(trifluoromethyl) phenyl]cyclobutyl}carbamate [20]

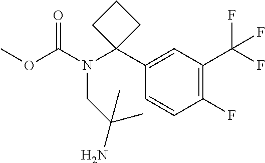 [SMILES: COC(=O)N(CC(C)(C)N)C1(c2ccc(F)c(C(F)(F)F)c2)CCC1]

### Step 1

To a solution of [16.4] (0.25 g, 1.1 mmol) in DCM (3 mL) and isopropanol (2 mL) at 0° C. was added tert-butyl 2-formylpropan-2-ylcarbamate (0.20 g, 1.1 mmol) and the reaction stirred for 15 min. Sodium triacetoxyborohydride (0.34 g, 1.6 mmol) was added in portions at 0° C. and the reaction stirred at RT for 2h. Water was added and the aqueous solution extracted twice with DCM. The combined organic layers were washed with water and brine solution, dried over sodium sulfate and concentrated to afford crude product which was purified by combi flash column by eluting with 15% ethyl acetate in hexane to afford the product [20.1] (0.17 g, 39%).

### Step 2

To a solution of [20.1] (0.13 g, 0.32 mmol) in DCM (2 mL) at 0° C. was added N,N-diisopropylethylamine (0.11 mL, 0.08 g, 0.64 mmol) and methyl chloroformate (0.06 g, 0.64 mmol) and the reaction stirred at RT for 12h. Water was added and the aqueous solution extracted twice with DCM. The combined organic layers were washed with brine solution, dried over sodium sulphate, filtered and evaporated under high vacuum to get crude product which was purified by prep-TLC to afford the product [20.2] (0.02 g, 14%).

### Step 3

To a solution of [20.2] (0.02 g, 0.04 mmol) in DCM (1 mL) at 0° C. was added methanolic HCl (3M, 0.03 mL) and the reaction stirred at RT for 2h. The reaction was concentrated under vacuum, the solid residue triturated with hexane and the solid collected by filtration to afford product [20] as hydrochloride salt (0.008 g, 46%).

¹H NMR (400 MHz, DMSO-d₆) δ ppm 1.08-1.11 (m, 2H), 1.33 (s, 6H), 1.46-1.59 (m, 1H), 1.70-1.80 (m, 1H), 2.5-2.63 (m, 2H), 3.10-3.17 (m, 1H), 3.54 (s, 3H), 3.59 (s, 1H), 7.40-7.60 (m, 1H), 7.69-7.84 (m, 1H), 7.81-7.99 (m, 5H).

### Example 21

### 1-[3-fluoro-5-(trifluoromethyl) phenyl]-N-{[(2R)-pyrrolidin-2-yl]methyl}cyclobutan-1-amine [21]

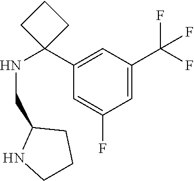 [SMILES: Fc1cc(C(F)(F)F)cc(C2(NC[C@H]3CCCN3)CCC2)c1]

### Step 1

To a solution of potassium hydroxide (6.96 g, 124 mmol) in water (20 mL) and toluene (1 mL) at 50° C. was added 1,3-dibromopropane (4.9 g, 24 mmol) and tetrabutylammonium bromide (0.36 g, 1.1 mmol). A solution of 3-fluoro-5-(trifluoromethyl)benzeneacetonitrile (4.5 g, 22.2 mmol) in toluene (1 mL) was slowly added to the reaction and the temperature raised to 100° C. and stirred for 2h. After cooling, the reaction mixture was extracted twice with DCM and the combined organic layers were washed with brine solution, dried over sodium sulphate, evaporated under high vacuum to get crude compound which was purified by using combi flash column by eluting with 5% ethyl acetate in hexane to afford [21.1] (2 g, 37%).

### Step 2

To a solution of [21.1] (2 g, 8.2 mmol) in acetic acid (8 mL) was added dropwise conc. sulfuric acid (4 mL) at 0° C. The temperature was raised to 90° C. and the reaction stirred for 16h. Ice cold water was added to the reaction mixture and the aqueous solution extracted twice with DCM. The combined organic layers were washed with sodium bicarbonate solution and brine solution, dried over sodium sulphate, evaporated under high vacuum to afford crude compound [21.2] (3.0 g) which was used without further purification.

### Step 3

To a solution of [21.2] (3 g, 11.5 mmol) in n-butanol (20 mL) at 0° C. was slowly added aq. NaOH solution (3M, 15 mL) and aq. sodium hypochlorite solution (6.7%, 7 mL) and the reaction stirred at RT for 12h. The reaction mass was diluted with water and extracted with ethyl acetate. The organic layer was washed with water and brine solution, dried over sodium sulfate and concentrated to afford crude product which was purified by using combi flash column by eluting with 15% ethyl acetate in hexane to afford [21.3] as a colourless liquid (0.7 g, 26%).

### Step 4

To a solution of [21.3] (0.25 g, 1.1 mmol) in DCM (6 mL) and isopropanol (4 mL) at 0° C. was added tert-butyl (R)-2-formylpyrrolidine-1-carboxylate (0.21 g, 1.1 mmol) and the reaction stirred for 10 min. Sodium triacetoxyborohydride (0.34 g, 1.61 mmol) was added in portions and the reaction stirred at RT for 2h. Water was added and the aqueous solution extracted twice with DCM. The combined organic layers were washed with water and brine solution, dried over sodium sulfate and concentrated to afford crude product which was purified by using combi flash column by eluting with ethyl acetate in hexane to afford [21.4] as a colourless liquid (0.25 g, 56%).

### Step 5

To a stirred solution of [21.4] (0.1 g, 0.24 mmol) in methanol (5 mL) at 0° C. was added methanolic HCl (3M, 2 mL) and the reaction stirred for 2h. The reaction was concentrated under vacuum, triturated with hexane and the solid collected by filtration to afford product [21] as hydrochloride salt (50 mg, 53%).

¹H NMR (300 MHz, DMSO-d₆) δ ppm 1.50-1.94 (m, 4H), 2.03-2.24 (m, 2H), 2.57-2.78 (m, 2H), 2.79-3.04 (m, 3H), 3.10-3.35 (m, 3H), 3.79-3.96 (m, 1H), 7.65-8.04 (m, 3H), 9.25 (bs, 1H), 9.40-9.48 (bs, 1H), 10.61 (bs, 2H).

### Example 22

### Methyl N-{1-[3-fluoro-5-(trifluoromethyl)phenyl]cyclobutyl}-N-{[(2S)-pyrrolidin-2-yl]methyl}carbamate [22]

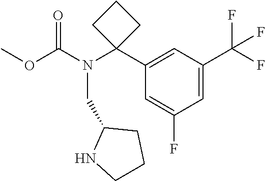 [SMILES: COC(=O)N(C[C@@H]1CCCN1)C1(c2cc(F)cc(C(F)(F)F)c2)CCC1]

### Step 1

To a solution of [21.3] (0.25 g, 1.1 mmol) in DCM (3 mL) and isopropanol (2 mL) at 0° C. was added tert-butyl (S)-2-formylpyrrolidine-1-carboxylate (0.24 g, 1.2 mmol) and the reaction stirred for 10 min. Sodium triacetoxyborohydride (0.34 g, 1.61 mmol) was added in portions and the reaction stirred at RT for 2h. Water was added and the aqueous solution extracted twice with DCM. The combined organic layers were washed with water and brine solution, dried over sodium sulfate and concentrated to afford crude product which was purified by using combi flash column by eluting with ethyl acetate in hexane to afford [22.1] as a colourless liquid (0.25 g, 56%).

### Step 2

To a solution of [22.1] (0.15 g, 0.36 mmol) in DCM (10 mL) at 0° C. was added N,N-diisopropylethylamine (0.31 mL, 0.23 g, 0.1.8 mmol) and methyl chloroformate (0.10 g, 1.1 mmol) and the reaction stirred at RT for 2h. Water was added and the aqueous solution extracted twice with DCM. The combined organic layers were washed with brine solution, dried over sodium sulphate, filtered and evaporated under high vacuum to get crude product which was purified combi flash column eluting with 10% ethyl acetate in hexane to afford product [22.2] (150 mg, 88%).

### Step 3

To a solution of [22.2] (150 mg, 0.32 mmol) in DCM (5 mL) and methanol (5 mL) at 0° C. was added methanolic HCl (3M, 2 mL) and the reaction stirred at RT for 2h. The reaction was concentrated under vacuum, triturated with hexane and the solid collected by filtration to afford product [22] as hydrochloride salt (100 mg, 84%).

¹H NMR (400 MHz, DMSO-d₆) δ ppm 1.41-1.69 (m, 2H), 1.75-2.03 (m, 3H), 1.99-2.19 (m, 1H), 2.53-2.77 (m, 3H), 3.00-3.15 (m, 1H), 3.22-3.31 (m, 1H), 3.31-3.50 (m, 2H), 3.53 (s, 3H), 3.56-3.84 (m, 2H), 7.49-7.85 (m, 3H), 8.70 (bs, 1H), 9.60 (bs, 1H).

### Example 23

### Methyl N-{1-[3-fluoro-5-(trifluoromethyl)phenyl]cyclobutyl}-N-{[(2R)-pyrrolidin-2-yl]methyl}carbamate [23]

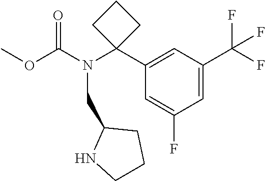 [SMILES: COC(=O)N(C[C@H]1CCCN1)C1(c2cc(F)cc(C(F)(F)F)c2)CCC1]

### Step 1

To a solution of [21.4] (0.15 g, 0.36 mmol) in DCM (10 mL) at 0° C. was added N,N-diisopropylethylamine (0.31 mL, 0.23 g, 0.1.8 mmol) and methyl chloroformate (0.10 g, 1.1 mmol) and the reaction stirred at RT for 2h. Water was added and the aqueous solution extracted twice with DCM. The combined organic layers were washed with brine solution, dried over sodium sulphate, filtered and evaporated under high vacuum to get crude product which was purified combi flash column eluting with 10% ethyl acetate in hexane to afford product [23.1] (150 mg, 88%).

### Step 2

To a solution of [23.1] (0.150 g, 0.32 mmol) in DCM (5 mL) and methanol (5 mL) at 0° C. was added methanolic HCl (3M, 2 mL) and the reaction stirred at RT for 2h. The reaction was concentrated under vacuum, triturated with hexane and the solid collected by filtration to afford product [23] as hydrochloride salt (90 mg, 76%).

¹H NMR (400 MHz, DMSO-d₆) δ ppm 1.43-1.69 (m, 2H), 1.69-1.93 (m, 3H), 1.96-2.06 (m, 1H), 2.53-2.74 (m, 3H), 3.02-3.16 (m, 1H), 3.18-3.31 (m, 1H), 3.42-3.62 (m, 5H), 3.65-3.87 (m, 2H), 7.47-7.67 (m, 2H), 7.68-7.75 (m, 1H), 8.58 (bs, 1H), 9.46 (bs, 1H).

### Example 24

### Methyl N-(2-amino-2-methylpropyl)-N-{1-[3-fluoro-5-(trifluoromethyl)phenyl]cyclobutyl}carbamate [24]

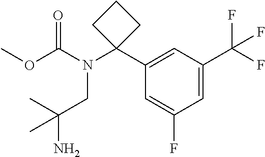 [SMILES: COC(=O)N(CC(C)(C)N)C1(c2cc(F)cc(C(F)(F)F)c2)CCC1]

### Step 1

To a solution of [21.3] (0.2 g, 0.86 mmol) in DCM (3 mL) and isopropanol (2 mL) at 0° C. was added tert-butyl 2-formylpropan-2-ylcarbamate (0.18 g, 0.94 mmol) and the reaction stirred for 2h. Sodium triacetoxyborohydride (0.27 g, 1.3 mmol) was added in portions at 0° C. and the reaction stirred at RT for 2h. Water was added and the aqueous solution extracted twice with DCM. The combined organic layers were washed with water and brine solution, dried over sodium sulfate and concentrated to afford crude product which was purified by column chromatography eluting with ethyl acetate in hexane to afford the product [24.1] (0.1 g, 28%).

### Step 2

To a solution of [24.1] (0.1 g, 0.25 mmol) in DCM (5 mL) at 0° C. was added N,N-diisopropylethylamine (0.22 mL, 0.16 g, 1.24 mmol) and methyl chloroformate (0.07 g, 0.74 mmol) and the reaction stirred at RT for 24h. Water was added and the aqueous solution extracted twice with DCM. The combined organic layers were washed with brine solution, dried over sodium sulphate, filtered and evaporated under high vacuum to get crude product which was purified by column chromatography eluting with ethyl acetate in hexane to afford the product [24.2] (0.1 g, 87%).

### Step 3

To a solution of [24.2] (0.100 g, 0.22 mmol) in DCM (5 mL) and methanol (5 mL) at 0° C. was added methanolic HCl (3M, 2 mL) and the reaction stirred at RT for 2h. The reaction was concentrated under vacuum and purified by prep-HPLC afford product [24] (0.015 g, 19%) as acetate salt.

¹H NMR (400 MHz, DMSO-d₆) δ ppm 1.12 (s, 6H), 1.49-1.63 (m, 1H), 1.69-1.83 (m, 1H), 2.51-2.61 (m, 3H), 2.62-2.73 (m, 3H), 3.44 (s, 3H), 7.55-7.63 (m, 1H), 7.63-7.69 (m, 1H), 7.69-7.82 (m, 1H), 8.36 (s, 1H).

### Example 25

### Methyl N-{1-[2-fluoro-5-(trifluoromethyl)phenyl]cyclobutyl}-N-{[(2S)-pyrrolidin-2-yl]methyl}carbamate [25]

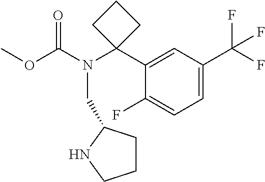 [SMILES: COC(=O)N(C[C@@H]1CCCN1)C1(c2cc(C(F)(F)F)ccc2F)CCC1]

### Step 1

To a solution of potassium hydroxide (14.7 g, 262 mmol) in water (4 mL) and toluene (40 mL) at 50° C. was added 1,3-dibromopropane (10.4 g, 51 mmol) and tetrabutylammonium bromide (0.76 g, 2.3 mmol). A solution of 2-fluoro-5-(trifluoromethyl)benzeneacetonitrile (9.5 g, 47 mmol) in toluene (4 mL) was slowly added to the reaction and the temperature raised to 100° C. and stirred for 2h. After cooling, the reaction mixture was extracted twice with DCM and the combined organic layers were washed with brine solution, dried over sodium sulphate, evaporated under high vacuum to get crude compound which was purified by using combi flash column by eluting with 5% ethyl acetate in hexane to afford [25.1] (4.5 g, 40%).

### Step 2

To a solution of [25.1] (4.5 g, 18.5 mmol) in acetic acid (18 mL) was added dropwise conc. sulfuric acid (9 mL) at 0° C. The temperature was raised to 90° C. and the reaction stirred for 16h. Ice cold water was added to the reaction mixture and the aqueous solution extracted twice with DCM. The combined organic layers were washed with sodium bicarbonate solution and brine solution, dried over sodium sulphate, evaporated under high vacuum to afford product [25.2] (4.5 g, 93%) which was used without further purification.

### Step 3

To a stirred solution of [25.2] (4.5 g, 17.2 mmol) in t-butanol (40 mL) at 55° C. was added lead tetraacetate (8.4 g, 19 mmol) in portions and the reaction stirred at 80° C. for 2h. The reaction mixture was diluted with EtOAc and filtered through celite. The filtrate was evaporated under high vacuum to get crude compound which was purified by using combi flash column by eluting with 5% ethyl acetate in hexane to afford the product [25.3] as a white solid (4 g, 70%).

### Step 4

To a solution of [25.3] (4 g, 12 mmol) in methanol (40 mL) was added methanolic HCl (3M, 20 mL) and the reaction stirred at RT for 2h. The reaction was concentrated under vacuum, triturated with hexane and the solid collected by filtration to afford product [25.4] as hydrochloride salt (2 g, 71%).

### Step 5

To a solution of [25.4] (0.25 g, 1.07 mmol) in DCM (6 mL) and isopropanol (4 mL) at 0° C. was added tert-butyl (S)-2-formylpyrrolidine-1-carboxylate (0.21 g, 1.07 mmol) and the reaction stirred for 10 min. Sodium triacetoxyborohydride (0.34 g, 1.6 mmol) was added in portions and the reaction stirred at RT for 2h. Water was added and the aqueous solution extracted twice with DCM. The combined organic layers were washed with water and brine solution, dried over sodium sulfate and concentrated to afford crude product which was purified by using combi flash column by eluting with 15% ethyl acetate in hexane to afford [25.5] (0.25 g, 56%).

### Step 6

To a solution of [25.5] (0.15 g, 0.36 mmol) in DCM (10 mL) at 0° C. was added N,N-diisopropylethylamine (0.31 mL, 0.23 g, 1.8 mmol) and methyl chloroformate (0.10 g, 1.08 mmol) and the reaction stirred at RT for 12h. Water was added and the aqueous solution extracted twice with DCM. The combined organic layers were washed with brine solution, dried over sodium sulphate, filtered and evaporated under high vacuum to get crude product [25.6] (0.15 g, 88%) which was used directly in the next step.

### Step 7

To a solution of [25.6] (0.100 g, 0.21 mmol) in DCM (5 mL) and methanol (5 mL) at 0° C. was added methanolic HCl (3M, 2 mL) and the reaction stirred at RT for 2h. The reaction was concentrated under vacuum, triturated with hexane and the solid collected by filtration to afford product [25] as hydrochloride salt (0.12 g, 92%).

¹H NMR (400 MHz, DMSO-d₆) δ ppm 1.47-1.61 (m, 1H), 1.66-1.73 (m, 1H), 1.77-2.04 (m, 4H), 2.07-2.24 (m, 1H), 2.58-2.81 (m, 2H), 3.04-3.22 (m, 2H), 3.23-3.34 (m, 1H), 3.45 (s, 3H), 3.57-3.80 (m, 3H), 7.37-7.56 (m, 1H), 7.68-7.81 (m, 1 H), 7.82-7.91 (m, 1H), 8.68 (bs, 1H), 9.48 (bs, 1H).

### Example 26

### Methyl N-[1-(5-chloro-2-fluorophenyl)cyclobutyl]-N-{[(2S)-pyrrolidin-2-yl]methyl}carbamate [26]

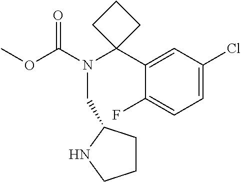 [SMILES: COC(=O)N(C[C@@H]1CCCN1)C1(c2cc(Cl)ccc2F)CCC1]

### Step 1

To a solution of potassium hydroxide (16.5 g, 294 mmol) in water (80 mL) and toluene (8 mL) at 50° C. was added 1,3-dibromopropane (13 g, 65 mmol) and tetrabutylammonium bromide (0.96 g, 3 mmol). A solution of 5-chloro-2-fluorobenzeneacetonitrile (10 g, 59 mmol) in toluene (8 mL) was slowly added to the reaction and the temperature raised to 90° C. and stirred for 1h. After cooling, the reaction mixture was extracted twice with DCM and the combined organic layers were washed with brine solution, dried over sodium sulphate, evaporated under high vacuum to get crude compound which was purified by using combi flash column by eluting with 5% ethyl acetate in hexane to afford [26.1] (6.5 g, 53%).

### Step 2

To a solution of [26.1] (6.5 g, 31 mmol) in acetic acid (26 mL) was added dropwise conc. sulfuric acid (13 mL) at 0° C. The temperature was raised to 90° C. and the reaction stirred for 16h. Ice cold water was added to the reaction mixture and the aqueous solution extracted twice with DCM. The combined organic layers were washed with sodium bicarbonate solution and brine solution, dried over sodium sulphate, evaporated under high vacuum to afford crude compound [26.2] (94% purity, 7 g, ˜95%) which was used without further purification.

### Step 3

To a stirred solution of [26.2] (0.5 g, 2.2 mmol) in t-butanol (5 mL) at 50° C. was added lead tetraacetate (1.07 g, 2.4 mmol) and the reaction stirred at 90° C. for 2h. The reaction mixture was diluted with EtOAc and filtered through celite. The filtrate was evaporated under high vacuum to get crude compound which was purified by using combi flash column by eluting with 5% ethyl acetate in hexane to afford the product [26.3] as a white solid (0.35 g, 53%).

### Step 4

To a solution of [26.3] (0.35 g, 1.17 mmol) in DCM (3 mL) was added methanolic HCl (3M, 6 mL) and the reaction stirred for 3h. The reaction was concentrated under vacuum, triturated with hexane and the solid collected by filtration to afford product [26.4] as hydrochloride salt (230 mg, 84%).

### Step 5

To a solution of [26.4] (0.24 g, 1 mmol) in DCM (3 mL) and isopropanol (2 mL) was added tert-butyl (S)-2-formylpyrrolidine-1-carboxylate (0.24 g, 1.2 mmol) and the reaction stirred for 15 min. Sodium triacetoxyborohydride (0.38 g, 1.8 mmol) was added and the reaction stirred at RT for 2h. Water was added and the aqueous solution extracted twice with DCM. The combined organic layers were washed with brine solution, dried over sodium sulphate, filtered and evaporated under high vacuum to get crude product [26.5] (380 mg, ˜99%) which was used without further purification.

### Step 6

To a solution of [26.5] (0.25 g, 0.65 mmol) in DCM (5 mL) at 0° C. was added N,N-diisopropylethylamine (0.228 mL, 0.17 g, 1.31 mmol) and methyl chloroformate (0.06 g, 0.65 mmol) and the reaction stirred at RT for 12h. Water was added and the aqueous solution extracted twice with DCM. The combined organic layers were washed with brine solution, dried over sodium sulphate, filtered and evaporated under high vacuum to get crude product which was purified by using combi flash column by eluting with 15% ethyl acetate in hexane to afford product [26.6] (120 mg, 42%).

### Step 7

To a stirred solution of [26.6] (0.12 g, 0.27 mmol) in DCM (1 mL) was added methanolic HCl (3M, 0.5 mL) and the reaction stirred for 3h. The reaction was concentrated under vacuum, triturated with hexane and the solid collected by filtration to afford product [26] as hydrochloride salt (72 mg, 72%).

¹H NMR (400 MHz, DMSO-d₆) δ ppm 1.19-1.37 (m, 1H), 1.48-1.61 (m, 1H), 1.62-1.73 (m, 1H), 1.75-2.00 (m, 3H), 2.06-2.08 (s, 1H), 2.11-2.18 (m, 1H), 2.55-2.75 (m, 2H), 3.04-3.22 (m, 1H), 3.26-3.31 (m, 1H), 3.33 (s, 3H), 3.54-3.64 (m, 1H), 3.68-3.71 (m, 2H), 7.26 (dd, J=11.40, 8.77 Hz, 1H), 7.34-7.48 (m, 1H), 7.61 (dd, J=7.02, 2.19 Hz, 1H), 8.64 (br s, 1H), 9.50 (br s, 1H).

### Example 27

### Methyl N-(2-amino-2-methylpropyl)-N-[1-(5-chloro-2-fluorophenyl)cyclobutyl]carbamate [27]

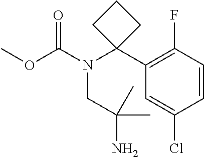 [SMILES: COC(=O)N(CC(C)(C)N)C1(c2cc(Cl)ccc2F)CCC1]

### Step 1

To a solution of [26.4] (0.3 g, 1.5 mmol) in DCM (3 mL) and isopropanol (2 mL) at 0° C. was added tert-butyl (S)-2-formylpyrrolidine-1-carboxylate (0.24 g, 1.2 mmol) and the reaction stirred for 30 min. Sodium triacetoxyborohydride (0.48 g, 2.25 mmol) was added in portions and the reaction stirred at RT for 12h. Water was added and the aqueous solution extracted twice with DCM. The combined organic layers were washed with water and brine solution, dried over sodium sulfate and concentrated to afford crude product which was purified by using combi flash column by eluting with 5% ethyl acetate in hexane to afford [27.1] as a colourless liquid (0.18 g, 32%).

### Step 2

To a solution of [27.1] (0.13 g, 0.35 mmol) in DCM (2 mL) at 0° C. was added N,N-diisopropylethylamine (0.12 mL, 0.09 g, 0.7 mmol) and methyl chloroformate (0.07 g, 0.7 mmol) and the reaction stirred at RT for 12h. Water was added and the aqueous solution extracted twice with DCM. The combined organic layers were washed with brine solution, dried over sodium sulphate, filtered and evaporated under high vacuum to get crude product which was purified by prep-TLC to afford the product [27.2] (0.04 g, 27%).

### Step 3

To a solution of [27.2] (0.03 g, 0.07 mmol) in DCM (0.5 mL) was added methanolic HCl (3M, 0.02 mL) and the reaction stirred at RT for 3h. The reaction was concentrated under vacuum, the solid residue triturated with hexane and the solid collected by filtration to afford product [27] as hydrochloride salt (0.007 g, 27%).

¹H NMR (400 MHz, METHANOL-d₄) δ ppm 1.28-1.32 (m, 1H), 1.48 (s, 6H) 1.62-1.73 (m, 1H) 1.82-1.97 (m, 1H) 2.51-2.75 (m, 4H) 3.56 (s, 3H), 3.59-3.85 (m, 1H) 7.08-7.21 (m, 1H) 7.28-7.44 (m, 1H) 7.56-7.71 (m, 1H).

### Example 28

### 1-(3-cyclopropyl-4-fluorophenyl)-N-{[(2R)-pyrrolidin-2-yl]methyl}cyclobutan-1-amine [28]

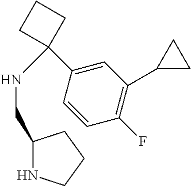 [SMILES: Fc1ccc(C2(NC[C@H]3CCCN3)CCC2)cc1C1CC1]

### Step 1

To a solution of 3-bromo-4-fluorophenylacetonitrile (5 g, 23 mmol) in THF (50 mL) was added sodium hydride (60%, 1.12 g, 28 mmol) in portions at 0° C. under nitrogen. After 15 min, 1,3-dibromopropane (5.2 g, 26 mmol) was added and the reaction stirred for 4h. The reaction mixture was diluted with water and extracted with ethyl acetate. The combined organics were washed with water and brine solution, dried over sodium sulphate, filtered and concentrated under reduced pressure to afford crude product as a pale brown liquid, which was purified by combi flash column by eluting with 5% ethyl acetate in hexane to afford [28.1] as a colourless liquid (1 g, 17%).

### Step 2

To a solution of [28.1] (2.9 g, 11.4 mmol) in acetic acid (12 mL) was added dropwise conc. sulfuric acid (6 mL) at 0° C. The temperature was raised to 90° C. and the reaction stirred for 10h. Ice cold water was added to the reaction mixture and the aqueous solution extracted twice with ethyl acetate. The combined organic layers were washed with water and brine solution, dried over sodium sulfate and concentrated under high vacuum to afford product [28.2] (2.8 g, 90%) which was used without further purification.

### Step 3

To a stirred solution of [28.2] (2.7 g, 9.9 mmol) in t-butanol (50 mL) at 50° C. was added lead tetraacetate (4.8 g, 11 mmol) in portions and the reaction refluxed for 2h. The reaction mixture was diluted with EtOAc and filtered through celite. The filtrate was evaporated under high vacuum to get crude compound which was purified by using combi flash column by eluting with 5% ethyl acetate in hexane to afford the product [28.3] as an off-white solid (1.8 g, 57%).

### Step 4

To a stirred solution of [28.3] (1.8 g, 5.2 mmol) in toluene (40 mL) and water (1 mL) were added potassium cyclopropyltrifluoroborate (0.93 g, 6.28 mmol), potassium carbonate (1.45 g, 10.5 mmol) and Pd(dppf)Cl₂ (0.38 g, 0.05 mmol). The solution was degassed with argon for 15 min. then the reaction was heated to 100° C. for 15h. The reaction was diluted with water and extracted with ethyl acetate. The combined organics were wased with water and brine solution, dried over sodium sulfate, filtered and concentrated under vacuum to obtain the crude product which was purified by combi flash column with 10% ethyl acetate in hexane to afford the product [28.4] as a white solid (0.45 g, 28%).

### Step 5

To a solution of [28.4] (0.45 g, 1.5 mmol) in DCM (10 mL) was added methanolic HCl (3M, 6 mL) at 0° C. and the reaction stirred at RT for 3h. The reaction was concentrated under vacuum, triturated with ether and pentane and the solid collected by filtration to afford product [28.5] as hydrochloride salt (0.32 g, 88%).

### Step 6

To a solution of [28.5] (0.1 g, 0.41 mmol) in DCM (6 mL) and isopropanol (4 mL) at 0° C. was added tert-butyl (R)-2-formylpyrrolidine-1-carboxylate (0.10 g, 0.49 mmol) and the reaction stirred for 15 min. Sodium triacetoxyborohydride (0.21 g, 0.98 mmol) was added in portions at 0° C. and the reaction stirred at RT for 4h. Water was added and the aqueous solution extracted twice with DCM. The combined organic layers were washed with water and brine solution, dried over sodium sulfate and concentrated to afford crude product [28.6] (0.13 g, 82%).

### Step 7

To a solution of [28.6] (0.05 g, 0.13 mmol) in DCM (5 mL) was added methanolic HCl (3M, 0.5 mL) at 0° C. and the reaction stirred at RT for 2h. The reaction was concentrated under vacuum, the residue triturated with ether and pentane and the solid collected by filtration to afford product [28] as hydrochloride salt (0.011 g, 29%).

¹H NMR (400 MHz, METHANOL-d₄) δ ppm 0.80-0.97 (m, 2H), 1.00-1.13 (m, 2H), 1.59-1.77 (m, 1H), 1.80-1.91 (m, 1H), 1.91-2.41 (m, 5H), 2.64-3.14 (m, 7H), 3.74-3.97 (m, 1H), 7.09-7.33 (m, 2H), 7.37-7.53 (m, 1H).

### Example 29

### 1-(3-cyclopropyl-4-fluorophenyl)-N-{[(2S)-pyrrolidin-2-yl]methyl}cyclobutan-1-amine [29]

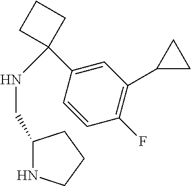 [SMILES: Fc1ccc(C2(NC[C@@H]3CCCN3)CCC2)cc1C1CC1]

### Step 1

To a solution of [28.5] (0.1 g, 0.41 mmol) in DCM (6 mL) and isopropanol (4 mL) at 0° C. was added tert-butyl (S)-2-formylpyrrolidine-1-carboxylate (0.10 g, 0.49 mmol) and the reaction stirred for 15 min. Sodium triacetoxyborohydride (0.21 g, 0.98 mmol) was added in portions at 0° C. and the reaction stirred at RT for 4h. Water was added and the aqueous solution extracted twice with DCM. The combined organic layers were washed with water and brine solution, dried over sodium sulfate and concentrated to afford crude product [29.1] (0.13 g, 82%).

### Step 2

To a solution of [29.1] (0.05 g, 0.13 mmol) in DCM (5 mL) was added methanolic HCl (3M, 0.5 mL) at 0° C. and the reaction stirred at RT for 2h. The reaction was concentrated under vacuum, the residue triturated with ether and pentane and the solid collected by filtration to afford product [29] as hydrochloride salt (0.025 g, 54%).

¹H NMR (300 MHz, METHANOL-d₄) δ ppm 0.76-0.96 (m, 2H), 0.96-1.16 (m, 2H), 1.55-1.71 (m, 1H), 1.77-1.89 (m, 1H), 1.93-2.35 (m, 6H), 2.56-2.87 (m, 5H), 2.89-3.16 (m, 2H), 3.68-3.93 (m, 1H), 7.07-7.31 (m, 2H), 7.37-7.46 (m, 1H).

### Example 30—Solubility of Compounds

The aim of this experiment was to determine solubility of test compounds in 50 mM Phosphate buffer by using HPLC.

Method

| | |
|---|---|
| Incubation time | 16 hr at ~25° C. |
| Buffer pH | 50 mM potassium phosphate buffer, pH 7.40 |
| Test compound Incubation | 1600 μM |
| concentration | |
| Replicates | n = 2 |
| Analysis | HPLC |
| Standard compounds | Caffeine [Solubility (1400-1900 μM)], Diethylstilbestrol |
| | [Solubility (0 μM)] and Tamoxifen [Solubility (<20 μM)] |
| Deliverables | Solubility of test compound mg/mL |

### Preparation of Phosphate Buffer (pH 7.4)

2.79 g of K₂HPO₄ and 0.54 g of KH₂PO₄ was dissolved in 390 mL of milliQ water. pH was adjusted to 7.4 using 1N HCl/1N NaOH and final volume was made up to 400 mL with milliQ water.

### Preparation and Dilution of Test Compound

Test compounds were prepared as described herein or in PCT/EP2020/057816.80 mM master stock of test compounds was prepared in 100% DMSO. In case of compounds not soluble/less quantity submission, 40/20/10Mm stocks were prepared and used for experiment.

Assay Procedure:
245 μL of 50 mM phosphate buffer then 5 μL each of test compound/standards (80 mM) in their respective positions was added to the 1.1 mL 96 well plate.
DMSO Controls was prepared by taking 245 μL of 100% DMSO then 5 μL each of test compound in their respective positions and added to the 1.1 mL 96 well plate
Plate was incubated with mixing at 1600 RPM for 16 hours at room temperature (˜23 MC).
After incubation, samples were filtered using Millipore plates.
Filtrates were analysed by HPLC-UV.

Solubility Calculation:

Solubility is calculated using the following formula:

$$Solubility ( µM ) = \frac{( Sample area in Buffer )}{( Sample area in DMSO )} \times 1600$$

Results

| | Measured | |
|---|---|---|
| | Solubility in pH 7.4 | |
| | phosphate buffer | |
| Compound | (mg/ml) | (μM) |
| Methyl N-(2-(dimethylamino)ethyl)-N-(1-(4-fluoro-3- | 0.5409 | 1600 |
| (trifluoromethyl)phenyl)cyclopropyl) carbamate | | |
| Methyl (2-amino-2-methylpropyl)(1-(4-fluoro-3- | 0.5555 | 1600 |
| (trifluoromethyl)phenyl)cyclopropyl)carbamate | | |
| Methyl ((1-aminocyclopropyl)methyl)(1-(4-fluoro-3- | 0.5599 | 1600 |
| (trifluoromethyl)phenyl)cyclopropyl) carbamate | | |
| Methyl 3-((1-(4-fluoro-3-(trifluoromethyl) phenyl) cyclopropyl) | 0.0279 | 100 |
| (methoxycarbonyl)amino)azetidine-1-carboxylate | | |
| N-(cyclopropylmethyl)-N-(1-(4-fluoro-3- | 0.6792 | 1500 |
| (trifluoromethyl)phenyl)cyclopropyl)azetidin-3-amine | | |
| N1-(cyclopropyl methyl)-N1-(1-(4-fluoro-3- | 0.5109 | 1500 |
| (trifluoromethyl)phenyl)cyclopropyl)-2-methyl propane-1,2- | | |
| diamine | | |
| N-(2-amino-2-methylpropyl)-N-(1-(4-fluoro-3- | 0.582 | 1200 |
| (trifluoromethyl)phenyl)cyclopropyl)cyclopropanecarboxamide | | |
| N1-cyclopropyl-2-methyl-N1-(1-(3- | 0.4809 | 1400 |
| (trifluoromethyl)phenyl)cyclopropyl)propane-1,2-diamine | | |
| Methyl (2-amino-2-methylpropyl)(1-(3,5- | 0.5778 | 1600 |
| dichlorophenyl)cyclopropyl)carbamate | | |
| Methyl (2-amino-2-methylpropyl)(1-(3- | 0.3755 | 800 |
| bromophenyl)cyclopropyl)carbamate | | |
| Methyl (2-amino-2-methylpropyl)(1-(3- | 0.6337 | 1500 |
| chlorophenyl)cyclopropyl)carbamate | | |
| 1-((azetidin-2-ylmethyl)(1-(4-fluoro-3-(trifluoromethyl) | 0.7554 | 1600 |
| phenyl)cyclopropyl)amino)-2-methyl propan-2-ol | | |
| Methyl (2-amino-2-methylpropyl)(1-(3- | 0.526 | 1400 |
| (trifluoromethyl)phenyl)cyclopropyl)carbamate | | |
| 1-((1-(4-fluoro-3-(trifluoromethyl) phenyl)cyclopropyl)((1- | 0.5974 | 1600 |
| methylazetidin-2-yl)methyl)amino)-2-methylpropan-2-ol | | |
| Methyl (2-amino-2-methylpropyl)(1-(5-fluoro-4- | 0.5636 | 1500 |
| (trifluoromethyl)pyridin-2-yl)cyclopropyl) carbamate | | |
| Ethyl (2-amino-2-methylpropyl)(1-(4-fluoro-3- | 0.607 | 1500 |
| (trifluoromethyl)phenyl)cyclopropyl)carbamate | | |
| Methyl (2-amino-2-methylpropyl)(1-(4-fluoro-3- | 0.5836 | 1500 |
| (trifluoromethoxy)phenyl)cyclopropyl)carbamate | | |
| N-(2-amino-2-methylpropyl)-N-(1-(4-fluoro-3- | 0.5646 | 1500 |
| (trifluoromethyl)phenyl)cyclopropyl)methanesulfonamide | | |
| Methyl (R)-(1-(4-fluoro-3- | 0.6666 | 1700 |
| (trifluoromethyl)phenyl)cyclopropyl)(pyrrolidin-2- | | |
| ylmethyl)carbamate | | |
| Methyl (S)-(1-(4-fluoro-3- | 0.653 | 1600 |
| (trifluoromethyl)phenyl)cyclopropyl)(pyrrolidin-2- | | |
| ylmethyl)carbamate | | |
| Methyl (2-acetamido-2-methylpropyl)(1-(4-fluoro-3- | 0.5958 | 1500 |
| (trifluoromethyl)phenyl)cyclopropyl)carbamate | | |
| N1-(1-(4-fluoro-3-(trifluoromethyl)phenyl) cyclopropyl)-2- | 0.4824 | 1700 |
| methylpropane-1,2-diamine | | |
| Methyl (azetidin-2-ylmethyl)(1-(4-fluoro-3- | 0.6868 | 1500 |
| (trifluoromethyl)phenyl)cyclopropyl)carbamate | | |
| Methyl (S)-(1-(4-fluoro-3-(trifluoro methyl) phenyl)cyclopropyl) | 0.5918 | 1600 |
| ((1-methylpyrrolidin-2-yl)methyl) carbamate | | |
| Methyl (S)-(1-(4-fluoro-3- | 0.6738 | 1600 |
| (trifluoromethoxy)phenyl)cyclopropyl)(pyrrolidin-2- | | |
| ylmethyl)carbamate | | |
| Methyl (R)-(1-(4-fluoro-3- | 0.6773 | 1600 |
| (trifluoromethoxy)phenyl)cyclopropyl)(pyrrolidin-2- | | |
| ylmethyl)carbamate | | |
| Methyl (1-(4-fluoro-3-(trifluoromethyl)phenyl)cyclopropyl)((1- | 0.5984 | 1700 |
| methylazetidin-2-yl)methyl)carbamate | | |
| Methyl (R)-(1-(4-fluoro-3- | 0.5575 | 1500 |
| (trifluoromethyl)phenyl)cyclopropyl)((1-methylpyrrolidin-2- | | |
| yl)methyl)carbamate | | |
| Methyl (azetidin-2-ylmethyl)(1-(4-fluoro-3- | 0.5578 | 1500 |
| (trifluoromethoxy)phenyl)cyclopropyl)carbamate | | |
| Methyl (1-(4-fluoro-3-(trifluoromethyl) phenyl)cyclopropyl)(2- | 0.1375 | 400 |
| (hydroxyamino)-2-methylpropyl)carbamate | | |
| Ethyl (S)-(1-(4-fluoro-3- | 0.594 | 1400 |
| (trifluoromethyl)phenyl)cyclopropyl)(pyrrolidin-2- | | |
| ylmethyl)carbamate | | |
| Ethyl (S)-(1-(4-fluoro-3- | 0.5679 | 1500 |
| (trifluoromethyl)phenyl)cyclopropyl)((1-methylpyrrolidin-2- | | |
| yl)methyl) carbamate | | |
| (S)-N-(1-(4-fluoro-3-(trifluoromethyl)phenyl)cyclopropyl)-N- | 0.3218 | 800 |
| (pyrrolidin-2-ylmethyl) methanesulfonamide | | |
| (S)-N-(1-(4-fluoro-3-(trifluoromethyl)phenyl)cyclopropyl)-N- | 0.621 | 1600 |
| ((1-methylpyrrolidin-2-yl)methyl)methanesulfonamide | | |
| Ethyl (R)-(1-(4-fluoro-3- | 0.6157 | 1500 |
| (trifluoromethyl)phenyl)cyclopropyl)(pyrrolidin-2- | | |
| ylmethyl)carbamate | | |
| Methyl (S)-(1-(4-fluoro-3- | 0.6118 | 1500 |
| (trifluoromethyl)phenyl)cyclopropyl)(morpholin-3-ylmethyl) | | |
| carbamate | | |
| Methyl (S)-(1-(4-fluoro-3- | 0.5996 | 1500 |
| (trifluoromethyl)phenyl)cyclopropyl)((4-methylmorpholin-3- | | |
| yl)methyl)carbamate | | |
| Isopropyl (S)-(1-(4-fluoro-3- | 0.6148 | 1600 |
| (trifluoromethyl)phenyl)cyclopropyl)(pyrrolidin-2- | | |
| ylmethyl)carbamate | | |
| Cyclopropyl (S)-(1-(4-fluoro-3- | 0.6009 | 1600 |
| (trifluoromethyl)phenyl)cyclopropyl)(pyrrolidin-2- | | |
| ylmethyl)carbamate | | |
| Methyl (R)-(1-(4-fluoro-3- | 0.585 | 1400 |
| (trifluoromethyl)phenyl)cyclopropyl)((2-methyl pyrrolidin-2- | | |
| yl)methyl)carbamate | | |
| N-((1-amino cyclopropyl)methyl)-1-(4-fluoro-3- | 0.4635 | 1400 |
| (trifluoromethyl)phenyl)cyclopropan-1-amine | | |
| Methyl (S)-(1-(4-fluoro-3- | 0.5747 | 1400 |
| (trifluoromethyl)phenyl)cyclopropyl)((2-methylpyrrolidin-2- | | |
| yl)methyl)carbamate | | |
| (1S,2S)-N1-(1-(4-fluoro-3-(trifluoromethyl) | 0.2392 | 700 |
| phenyl)cyclopropyl)-cyclopentane-1,2-diamine | | |
| (1R,2S)-N1-(1-(4-fluoro-3- | 0.4797 | 1400 |
| (trifluoromethyl)phenyl)cyclopropyl)-cyclopentane-1,2- | | |
| diamine | | |
| Methyl (S)-(azetidin-2-ylmethyl)(1-(4-fluoro-3- | 0.5907 | 1700 |
| (trifluoromethyl)phenyl)-cyclopropyl)carbamate | | |
| NS6180 | 0.0009 | 2.8 |

### Conclusion

It is demonstrated that the tested compounds have solubility in pH 7.4 phosphate buffer of 400 to 1700 μM, whereas NS6180 has a solubility of 2.8 μM.

### Example 31—Inhibition of K_{Ca}3.1

Test compounds were prepared as described herein or in PCT/EP2020/057816.

Erythrocyte K_{Ca}3.1 Assay

Human blood was drawn from healthy human volunteers in a standard heparinized blood sampling vial (Vacutainer, Li/heparin, ED Bioscience, Plymouth, UK). The erythrocytes were packed by centrifugation, and the plasma and buffy coat were removed by aspiration. Erythrocytes were washed three times in the experimental salt solution and then stored at 0° C. until use. Blood samples from NMRI mice or from Wistar rats were treated similarly. The methodological principle is outlined in Macey et al. (1978) and further described in Strøbaek et al. (2013). Activation of the erythrocyte K_{Ca}3.1 channels were obtained by addition of the Ca²⁺ ionophore A23187, which causes synchronized hyperpolarization, which is reported as a CCCP-mediated shift in the unbuffered extracellular pH of the erythrocyte suspension. Standard procedure: 3 mL unbuffered experimental salt solution (in mM: 2 KCl, 154 NaCl, 0.05 CaCl₂) was heated to 37° C. with stirring. Packed erythrocytes were added (50 μL, final cytocrit 1.5%), and the extracellular pH (pHₒ) followed with a glass/calomel (pHG200-8/REF200, Radiometer, Denmark) electrode pair. CCCP (3 μL, final concentration 20 μM) was added followed by varying concentrations of test compounds (DMSO concentration constant). After pH stabilization at ˜7.2, A23187 (3 μL, final concentration 0.33 μM) was added to initiate the experiment. After the peak hyperpolarization was attained, the intracellular pH (pHᵢ constant during the experiment) was found by haemolysing the erythrocytes via addition of 100 μL of Triton-X100.

The erythrocyte membrane potential, Vₘ, was calculated according to:

Vₘ=−61.5mV×(pHₒ−pHᵢ)

and the fractional remaining Ca²⁺-activated K⁺-conductance at the concentration C of blocker, fG_{K}(C), was calculated from

$${fG}_{K} ( C ) = \frac{( {V}_{m} ( 0 ) - {E}_{K} ) * ( {E}_{Cl} - {V}_{m} ( C ) )}{( {E}_{Cl} - {V}_{m} ( 0 ) ) * ( {V}_{m} ( C ) - {E}_{K} )}$$

where the K⁺ equilibrium potential E_{K}=−107 mV, the Cl⁻ equilibrium potential E_{Cl}=−12 mV and the Vₘ(0) and Vₘ(C) are the peak hyperpolarizations in the control and in the presence of a concentration of C of blocker respectively.

IC₅₀ values for compounds were calculated from a plot of fG_{K}(C) versus C by a fit to the Hill equation, using a custom program written in the IGOR-Pro software (WaveMetrics, Lake Oswego, OR, USA). All IC₅₀-values are reported in μM.

Results

| | RBC K (in vitro) |
|---|---|
| | Human IC₅₀ (μM) |
| | Potency category |
| | * IC₅₀ < 1 μM |
| Compound | ** IC₅₀ < 0.3 μM |
| N-{[(2S)-pyrrolidin-2-yl]methyl}-1-[3- | * |
| (trifluoromethyl)phenyl]cyclobutan-1-amine | |
| N-{[(2R)-pyrrolidin-2-yl]methyl}-1-[3- | * |
| (trifluoromethyl)phenyl]cyclobutan-1- | |
| 2-methyl-N1-{1-[3-(trifluoromethyl)phenyl]cyclobutyl}propane- | ** |
| 1,2-diamine | |
| methyl N-{[(2S)-pyrrolidin-2-yl]methyl}-N-{1-[3- | ** |
| (trifluoromethyl)phenyl]cyclobutyl}carbamate | |
| methyl N-{[(2R)-pyrrolidin-2-yl]methyl}-N-{1-[3- | ** |
| (trifluoromethyl)phenyl]cyclobutyl}carbamate | |
| methyl N-(2-amino-2-methylpropyl)-N-{1-[3- | ** |
| (trifluoromethyl)phenyl]cyclobutyl}carbamate | |
| N1-[1-(3-chlorophenyl)cyclobutyl]-2-methylpropane-1,2- | ** |
| diamine | |
| Methyl N-[1-(3-chlorophenyl)cyclobutyl]-N-{[(2S)-pyrrolidin-2- | ** |
| yl]methyl}carbamate | |
| Methyl N-[1-(3-chlorophenyl)cyclobutyl]-N-{[(2R)-pyrrolidin-2- | ** |
| yl]methyl}carbamate | |
| N-{[(2S)-pyrrolidin-2-yl]methyl}-1-[3- | * |
| (trifluoromethoxy)phenyl]cyclobutan-1-amine | |
| N-{[(2R)-pyrrolidin-2-yl]methyl}-1-[3- | * |
| (trifluoromethoxy)phenyl]cyclobutan-1-amine | |
| Methyl N-{[(2S)-pyrrolidin-2-yl]methyl}-N-{1-[3- | ** |
| (trifluoromethoxy)phenyl]cyclobutyl}carbamate | |
| Methyl N-{[(2R)-pyrrolidin-2-yl]methyl}-N-{1-[3- | ** |
| (trifluoromethoxy)phenyl]cyclobutyl}carbamate | |
| Methyl N-(2-amino-2-methylpropyl)-N-{1-[3- | ** |
| (trifluoromethoxy)phenyl]cyclobutyl}carbamate | |
| 1-[4-fluoro-3-(trifluoromethyl)phenyl]-N-{[(2S)-pyrrolidin-2- | ** |
| yl]methyl}cyclobutan-1-amine | |
| 1-[4-fluoro-3-(trifluoromethyl)phenyl]-N-{[(2R)-pyrrolidin-2- | * |
| yl]methyl}cyclobutan-1-amine | |
| N1-{1-[4-fluoro-3-(trifluoromethyl)phenyl]cyclobutyl}-2- | ** |
| methylpropane-1,2-diamine | |
| methyl N-{1-[4-fluoro-3-(trifluoromethyl)phenyl]cyclobutyl}-N- | ** |
| {[(2S)-pyrrolidin-2-yl]methyl}carbamate | |
| methyl N-{1-[4-fluoro-3-(trifluoromethyl)phenyl]cyclobutyl}-N- | ** |
| {[(2R)-pyrrolidin-2-yl]methyl}carbamate | |
| methyl N-(2-amino-2-methylpropyl)-N-{1-[4-fluoro-3- | ** |
| (trifluoromethyl)phenyl]cyclobutyl}carbamate | |
| 1-[3-fluoro-5-(trifluoromethyl)phenyl]-N-{[(2R)-pyrrolidin-2- | * |
| yl]methyl}cyclobutan-1-amine | |
| Methyl N-{1-[3-fluoro-5-(trifluoromethyl)phenyl]cyclobutyl}-N- | ** |
| {[(2S)-pyrrolidin-2-yl]methyl}carbamate | |
| Methyl N-{1-[3-fluoro-5-(trifluoromethyl)phenyl]cyclobutyl}-N- | ** |
| {[(2R)-pyrrolidin-2-yl]methyl}carbamate | |
| Methyl N-(2-amino-2-methylpropyl)-N-{1-[3-fluoro-5- | ** |
| (trifluoromethyl)phenyl]cyclobutyl}carbamate | |
| Methyl N-{1-[2-fluoro-5-(trifluoromethyl)phenyl]cyclobutyl}-N- | * |
| {[(2S)-pyrrolidin-2-yl]methyl}carbamate | |
| Methyl N-[1-(5-chloro-2-fluorophenyl)cyclobutyl]-N-{[(2S)- | * |
| pyrrolidin-2-yl]methyl}carbamate | |
| Methyl N-(2-amino-2-methylpropyl)-N-[1-(5-chloro-2- | * |
| fluorophenyl)cyclobutyl]carbamate | |
| 1-(3-cyclopropyl-4-fluorophenyl)-N-{[(2R)-pyrrolidin-2- | * |
| yl]methyl}cyclobutan-1-amine | |
| 1-(3-cyclopropyl-4-fluorophenyl)-N-{[(2S)-pyrrolidin-2- | * |
| yl]methyl}cyclobutan-1-amine | |
| Methyl N-(2-(dimethylamino)ethyl)-N-(1-(4-fluoro-3- | * |
| (trifluoromethyl)phenyl)cyclopropyl) carbamate | |
| Methyl (2-amino-2-methylpropyl)(1-(4-fluoro-3- | ** |
| (trifluoromethyl)phenyl)cyclopropyl)carbamate | |
| Methyl ((1-aminocyclopropyl)methyl)(1-(4-fluoro-3- | ** |
| (trifluoromethyl)phenyl)cyclopropyl) carbamate | |
| Methyl 3-((1-(4-fluoro-3-(trifluoromethyl) phenyl) cyclopropyl) | ** |
| (methoxycarbonyl)amino)azetidine-1-carboxylate | |
| Methyl azetidin-3-yl(1-(4-fluoro-3- | * |
| (trifluoromethyl)phenyl)cyclopropyl)carbamate | |
| N-(cyclopropylmethyl)-N-(1-(4-fluoro-3- | ** |
| (trifluoromethyl)phenyl)cyclopropyl)azetidin-3-amine | |
| Methyl (2-aminoethyl)(1-(4-fluoro-3- | * |
| (trifluoromethyl)phenyl)cyclopropyl)carbamate | |
| N1-(cyclopropyl methyl)-N1-(1-(4-fluoro-3- | ** |
| (trifluoromethyl)phenyl)cyclopropyl)-2-methyl propane-1,2- | |
| diamine | |
| N-(2-amino-2-methylpropyl)-N-(1-(4-fluoro-3- | * |
| (trifluoromethyl)phenyl)cyclopropyl)cyclopropanecarboxamide | |
| N1-cyclopropyl-2-methyl-N1-(1-(3- | ** |
| (trifluoromethyl)phenyl)cyclopropyl)propane-1,2-diamine | |
| Methyl (2-amino-2-methylpropyl)(1-(3,5- | ** |
| dichlorophenyl)cyclopropyl)carbamate | |
| Methyl (2-amino-2-methylpropyl)(1-(3- | * |
| bromophenyl)cyclopropyl)carbamate | |
| Methyl (2-amino-2-methylpropyl)(1-(3- | * |
| chlorophenyl)cyclopropyl)carbamate | |
| 1-((azetidin-2-ylmethyl)(1-(4-fluoro-3-(trifluoromethyl) | ** |
| phenyl)cyclopropyl)amino)-2-methyl propan-2-ol | |
| Methyl (2-amino-2-methylpropyl)(1-(3- | ** |
| (trifluoromethyl)phenyl)cyclopropyl)carbamate | |
| 1-((1-(4-fluoro-3-(trifluoromethyl) phenyl)cyclopropyl)((1- | * |
| methylazetidin-2-yl)methyl)amino)-2-methylpropan-2-ol | |
| Methyl (2-amino-2-methylpropyl)(1-(5-fluoro-4- | * |
| (trifluoromethyl)pyridin-2-yl)cyclopropyl) carbamate | |
| N1-cyclobutyl-2-methyl-N1-(1-(3- | ** |
| (trifluoromethyl)phenyl)cyclopropyl)propane-1,2-diamine | |
| Ethyl (2-amino-2-methylpropyl)(1-(4-fluoro-3- | ** |
| (trifluoromethyl)phenyl)cyclopropyl)carbamate | |
| Methyl (2-amino-2-methylpropyl)(1-(4-fluoro-3- | ** |
| (trifluoromethoxy)phenyl)cyclopropyl)carbamate | |
| N-(2-amino-2-methylpropyl)-N-(1-(4-fluoro-3- | ** |
| (trifluoromethyl)phenyl)cyclopropyl)methanesulfonamide | |
| Methyl (R)-(1-(4-fluoro-3- | ** |
| (trifluoromethyl)phenyl)cyclopropyl)(pyrrolidin-2- | |
| ylmethyl)carbamate | |
| Methyl (S)-(1-(4-fluoro-3- | ** |
| (trifluoromethyl)phenyl)cyclopropyl)(pyrrolidin-2- | |
| ylmethyl)carbamate | |
| Methyl (2-acetamido-2-methylpropyl)(1-(4-fluoro-3- | * |
| (trifluoromethyl)phenyl)cyclopropyl)carbamate | |
| N1-(1-(4-fluoro-3-(trifluoromethyl)phenyl) cyclopropyl)-2- | ** |
| methylpropane-1,2-diamine | |
| Methyl (azetidin-2-ylmethyl)(1-(4-fluoro-3- | ** |
| (trifluoromethyl)phenyl)cyclopropyl)carbamate | |
| Methyl (S)-(1-(4-fluoro-3-(trifluoro methyl) phenyl)cyclopropyl) | ** |
| ((1-methylpyrrolidin-2-yl)methyl) carbamate | |
| Methyl (S)-(1-(4-fluoro-3- | ** |
| (trifluoromethoxy)phenyl)cyclopropyl)(pyrrolidin-2- | |
| ylmethyl)carbamate | |
| Methyl (R)-(1-(4-fluoro-3- | * |
| (trifluoromethoxy)phenyl)cyclopropyl)(pyrrolidin-2- | |
| ylmethyl)carbamate | |
| Methyl (1-(4-fluoro-3-(trifluoromethyl)phenyl)cyclopropyl)((1- | ** |
| methylazetidin-2-yl)methyl)carbamate | |
| Methyl (R)-(1-(4-fluoro-3- | ** |
| (trifluoromethyl)phenyl)cyclopropyl)((1-methylpyrrolidin-2- | |
| yl)methyl)carbamate | |
| Methyl (azetidin-2-ylmethyl)(1-(4-fluoro-3- | ** |
| (trifluoromethoxy)phenyl)cyclopropyl)carbamate | |
| Methyl (1-(4-fluoro-3-(trifluoromethyl) phenyl)cyclopropyl)(2- | ** |
| (hydroxyamino)-2-methylpropyl)carbamate | |
| Ethyl (S)-(1-(4-fluoro-3- | ** |
| (trifluoromethyl)phenyl)cyclopropyl)(pyrrolidin-2- | |
| ylmethyl)carbamate | |
| Ethyl (S)-(1-(4-fluoro-3-(trifluoromethyl)phenyl)cyclopropyl)((1- | ** |
| methylpyrrolidin-2-yl)methyl) carbamate | |
| (S)-N-(1-(4-fluoro-3-(trifluoromethyl)phenyl)cyclopropyl)-N- | * |
| (pyrrolidin-2-ylmethyl) methanesulfonamide | |
| (S)-N-(1-(4-fluoro-3-(trifluoromethyl)phenyl)cyclopropyl)-N-((1- | ** |
| methylpyrrolidin-2-yl)methyl)methanesulfonamide | |
| Ethyl (R)-(1-(4-fluoro-3- | ** |
| (trifluoromethyl)phenyl)cyclopropyl)(pyrrolidin-2- | |
| ylmethyl)carbamate | |
| Methyl (R)-(1-(4-fluoro-3- | * |
| (trifluoromethyl)phenyl)cyclopropyl)(morpholin-3-ylmethyl) | |
| carbamate | |
| Methyl (R)-(1-(4-fluoro-3-(trifluoromethyl) | ** |
| phenyl)cyclopropyl)((4-methylmorpholin-3-yl)methyl)carbamate | |
| (R)-N-(1-(4-fluoro-3-(trifluoromethyl)phenyl)cyclopropyl)-N- | * |
| (pyrrolidin-2-ylmethyl) methane sulfonamide | |
| Methyl (S)-(1-(4-fluoro-3- | * |
| (trifluoromethyl)phenyl)cyclopropyl)(morpholin-3-ylmethyl) | |
| carbamate | |
| Methyl (S)-(1-(4-fluoro-3- | ** |
| (trifluoromethyl)phenyl)cyclopropyl)((4-methylmorpholin-3- | |
| yl)methyl)carbamate | |
| Isopropyl (S)-(1-(4-fluoro-3- | ** |
| (trifluoromethyl)phenyl)cyclopropyl)(pyrrolidin-2- | |
| ylmethyl)carbamate | |
| Cyclopropyl (S)-(1-(4-fluoro-3- | ** |
| (trifluoromethyl)phenyl)cyclopropyl)(pyrrolidin-2- | |
| ylmethyl)carbamate | |
| Methyl (R)-(1-(4-fluoro-3- | ** |
| (trifluoromethyl)phenyl)cyclopropyl)((2-methyl pyrrolidin-2- | |
| yl)methyl)carbamate | |
| N-((1-amino cyclopropyl)methyl)-1-(4-fluoro-3- | ** |
| (trifluoromethyl)phenyl)cyclopropan-1-amine | |
| Cyclopropylmethyl (S)-(1-(4-fluoro-3- | ** |
| (trifluoromethyl)phenyl)cyclopropyl)(pyrrolidin-2- | |
| ylmethyl)carbamate | |
| N-((1-aminocyclopropyl)methyl)-N-(1-(4-fluoro-3- | * |
| (trifluoromethyl)-phenyl)cyclopropyl)methanesulfonamide | |
| Methyl (S)-(1-(4-fluoro-3- | ** |
| (trifluoromethyl)phenyl)cyclopropyl)((2-methylpyrrolidin-2- | |
| yl)methyl)carbamate | |
| (1S,2S)-N1-(1-(4-fluoro-3-(trifluoromethyl) | * |
| phenyl)cyclopropyl)-cyclopentane-1,2-diamine | |
| (1R,2S)-N1-(1-(4-fluoro-3-(trifluoromethyl)phenyl)cyclopropyl)- | ** |
| cyclopentane-1,2-diamine | |
| Methyl (S)-(azetidin-2-ylmethyl)(1-(4-fluoro-3- | * |
| (trifluoromethyl)phenyl)-cyclopropyl)carbamate | |

### CONCLUSION

It is demonstrated that all the compounds inhibit K_{Ca}3.1.

### REFERENCES

Macey et al., Biochim. Biophys. Acta 1978, 22, 512(2), 284-95
Strøbaek et al., Br. J. Pharmacol. 2013, 168(2), 432-444
WO 2014/001363 [Clevexel Pharma; Aniona ApS; Saniona ApS]
WO 2013/191984 [Boehringer Ingelheim]
WO 2014/067861 [Hoffmann La Roche]

1. A compound of formula (I): 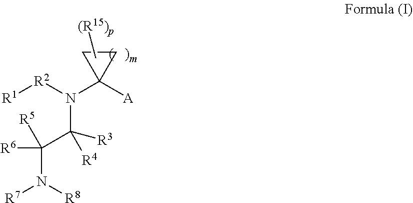
wherein
R¹ is —OC₁₋₈ alkyl; —C₁₋₈ alkyl, optionally substituted with —OH; or H;
R² is a bond; —C(O)—; —S(O)₂—; or —C(H)₂—;
R³ is H; C₁₋₅ alkyl; or a bond;
R⁴ is H; C₁₋₅ alkyl; or a bond;
R⁵ is H; a bond; or C₁₋₈ alkyl, wherein one methylene group optionally is replaced by —O—;
R⁶ is H; a bond; or C₁₋₈ alkyl, wherein one methylene group optionally is replaced by —O—;
R⁷ is H; a bond; —OH; or C₁₋₈ alkyl, wherein one or more methylene group optionally and individually is replaced by —O— and/or substituted with ═O;
R³ is H; a bond; —OH; or C₁₋₈ alkyl, wherein one or more methylene group optionally and individually is replaced by —O— and/or substituted with ═O;
R¹⁵ is individually selected from the group consisting of C₁₋₃ alkyl; —OH; —CN; and —F;
anyone of R³, R⁴, R⁵, R⁶, R⁷, and R³ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₃ alkyl, —OC₁₋₃ alkyl, —C₃₋₇ cycloalkyl, —OC₃₋₇ cycloalkyl, —CN, NO₂, —SO₂CH₃, and —SF₅;
X is halogen;
m is an integer of 1 to 3; and
p is an integer of 0 to 8;
or a pharmaceutically acceptable salt thereof,
with the proviso that p is not 0 when m is 1, and
with the proviso that the compound is not a compound selected from the group consisting of:
(2R)—N-[1-(4-fluorophenyl)cyclobutyl]-α-methyl-2-pyrrolidinemethanamine;
(2S)—N-[1-(4-fluorophenyl)cyclobutyl]-α-methyl-2-pyrrolidinemethanamine;
N1-[1-(3-bromophenyl)cyclobutyl]-1,2-ethanediamine;
N1-[1-(4-fluorophenyl)cyclobutyl]-1,2-ethanediamine;
N1-[1-(4-chlorophenyl)cyclobutyl]-1,2-ethanediamine;
N-(1-phenylcyclobutyl)-3-azetidinamine;
N1-[1-(4-bromophenyl)cyclobutyl]-1,2-ethanediamine;
(5S)-5-[[[1-(4-fluorophenyl)cyclopentyl]amino]methyl]-2-pyrrolidinone;
N1-[1-(4-fluorophenyl)cyclopentyl]-1,2-ethanediamine; and
N1-[1-(3-methylphenyl)cyclopentyl]-1,2-ethanediamine.

2. The compound according to claim 1, wherein the compound is of formula (IV): 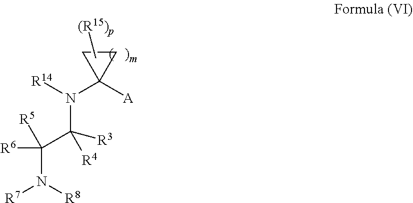
wherein
R¹⁴ is selected from the group consisting of —C(O)—C₁₋₈ alkyl; —C(O)—O—C₁₋₈ alkyl;
—C₂₋₈ alkyl; —H and —S(O)₂—C₁₋₈ alkyl;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R⁸ is H, a bond, —OH, or C₁₋₈ alkyl, wherein one or more methylene group optionally and individually is replaced by —O— and/or substituted with ═O;
R¹⁵ is individually selected from the group consisting of C₁₋₃ alkyl, —OH, —CN, and —F;
anyone of R³, R⁴, R⁵, R⁶, R⁷, and R⁸ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅; and
X is halogen;
m is an integer of 1 to 3; and
p is an integer of 0 to 8;
or a pharmaceutically acceptable salt thereof.

3. The compound according to any one of the preceding claims, wherein —R¹-R² is R¹⁴, and R¹⁴ is —C(O)—OC₁₋₄ alkyl.

4. The compound according to any one of the preceding claims, wherein A is a moiety of formula (X): 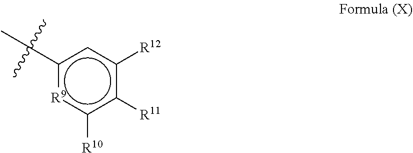
wherein
R⁹ is —C(H)—, —N—, or —C(R¹³)—;
R¹⁰, R¹¹, R¹², and R¹³ are individually selected from the group consisting of H, halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅; and
X is halogen.

5. The compound according to claim 4, wherein
R⁹ is —C(H)— or —N—;
R¹⁰ is H or halogen;
R¹¹ is H or halogen;
R¹² is —CX₃, —OCX₃, H, halogen, —C₁₋₈ alkyl, or —C₃₋₇ cycloalkyl; and
X is halogen.

6. The compound according to any one of the preceding claims, wherein R³ and R⁴ are —H, R⁵ and R⁶ are methyl, and R⁷ and R³ are —H.

7. The compound according to any one of the preceding claims, wherein the compound is of formula (VII):

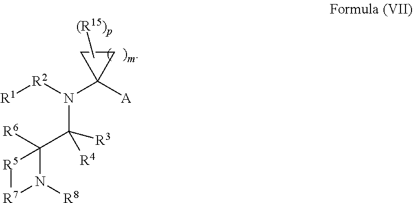

8. The compound according to any one of the preceding claims, wherein the compound is of formula (VIII):

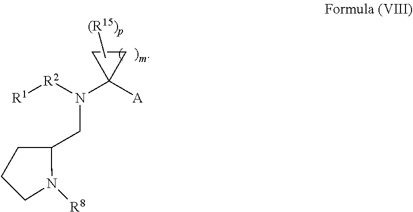

9. The compound according to any one of the preceding claims, wherein m is 2.

10. The compound according to any one of the preceding claims, wherein p is 0.

11. The compound according to any one of the preceding claims, wherein R¹² is —CF₃, —OCF₃, or a halogen.

12. The compound according to any one of the preceding claims, wherein R⁹ is —C(H)—, R¹⁰ is H, R¹¹ is F and R¹² is —CF₃.

13. The compound according to any one of the preceding claims, wherein the compound is selected from the group consisting of:
a. N-{[(2S)-pyrrolidin-2-yl]methyl}-1-[3-(trifluoromethyl)phenyl]cyclobutan-1-amine;
b. N-{[(2R)-pyrrolidin-2-yl]methyl}-1-[3-(trifluoromethyl)phenyl]cyclobutan-1-amine;
c. 2-methyl-N1-{1-[3-(trifluoromethyl)phenyl]cyclobutyl}propane-1,2-diamine;
d. methyl N-{[(2S)-pyrrolidin-2-yl]methyl}-N-{1-[3-(trifluoromethyl)phenyl]cyclobutyl}carbamate;
e. methyl N-{[(2R)-pyrrolidin-2-yl]methyl}-N-{1-[3-(trifluoromethyl)phenyl]cyclobutyl}carbamate;
f. methyl N-(2-amino-2-methylpropyl)-N-{1-[3-(trifluoromethyl)phenyl]cyclobutyl}carbamate;
g. N1-[1-(3-chlorophenyl)cyclobutyl]-2-methylpropane-1,2-diamine;
h. methyl N-[1-(3-chlorophenyl)cyclobutyl]-N-{[(2S)-pyrrolidin-2-yl]methyl}carbamate;
i. methyl N-[1-(3-chlorophenyl)cyclobutyl]-N-{[(2R)-pyrrolidin-2-yl]methyl}carbamate;
j. N-{[(2S)-pyrrolidin-2-yl]methyl}-1-[3-(trifluoromethoxy)phenyl]cyclobutan-1-amine;
k. N-{[(2R)-pyrrolidin-2-yl]methyl}-1-[3-(trifluoromethoxy)phenyl]cyclobutan-1-amine;
l. methyl N-{[(2S)-pyrrolidin-2-yl]methyl}-N-{1-[3-(trifluoromethoxy)phenyl]cyclobutyl}carbamate;
m. methyl N-{[(2R)-pyrrolidin-2-yl]methyl}-N-{1-[3-(trifluoromethoxy)phenyl]cyclobutyl}carbamate;
n. methyl N-(2-amino-2-methylpropyl)-N-{1-[3-(trifluoromethoxy)phenyl]cyclobutyl}carbamate;
o. 1-[4-fluoro-3-(trifluoromethyl)phenyl]-N-{[(2S)-pyrrolidin-2-yl]methyl}cyclobutan-1-amine;
p. 1-[4-fluoro-3-(trifluoromethyl)phenyl]-N-{[(2R)-pyrrolidin-2-yl]methyl}cyclobutan-1-amine;
q. N1-{1-[4-fluoro-3-(trifluoromethyl)phenyl]cyclobutyl}-2-methylpropane-1,2-diamine;
r. methyl N-{1-[4-fluoro-3-(trifluoromethyl)phenyl]cyclobutyl}-N-{[(2S)-pyrrolidin-2-yl]methyl}carbamate;
s. methyl N-{1-[4-fluoro-3-(trifluoromethyl)phenyl]cyclobutyl}-N-{[(2R)-pyrrolidin-2-yl]methyl}carbamate;
t. methyl N-(2-amino-2-methylpropyl)-N-{1-[4-fluoro-3-(trifluoromethyl)phenyl]cyclobutyl}carbamate;
u. 1-[3-fluoro-5-(trifluoromethyl)phenyl]-N-{[(2R)-pyrrolidin-2-yl]methyl}cyclobutan-1-amine;
v. methyl N-{1-[3-fluoro-5-(trifluoromethyl)phenyl]cyclobutyl}-N-{[(2S)-pyrrolidin-2-yl]methyl}carbamate;
w. methyl N-{1-[3-fluoro-5-(trifluoromethyl)phenyl]cyclobutyl}-N-{[(2R)-pyrrolidin-2-yl]methyl}carbamate;
x. methyl N-(2-amino-2-methylpropyl)-N-{1-[3-fluoro-5-(trifluoromethyl)phenyl]cyclobutyl}carbamate;
y. methyl N-{1-[2-fluoro-5-(trifluoromethyl)phenyl]cyclobutyl}-N-{[(2S)-pyrrolidin-2-yl]methyl}carbamate;
z. methyl N-[1-(5-chloro-2-fluorophenyl)cyclobutyl]-N-{[(2S)-pyrrolidin-2-yl]methyl}carbamate;
aa. methyl N-(2-amino-2-methylpropyl)-N-[1-(5-chloro-2-fluorophenyl)cyclobutyl]carbamate;
bb. 1-(3-cyclopropyl-4-fluorophenyl)-N-{[(2R)-pyrrolidin-2-yl]methyl}cyclobutan-1-amine; and
cc. 1-(3-cyclopropyl-4-fluorophenyl)-N-{[(2S)-pyrrolidin-2-yl]methyl}cyclobutan-1-amine.

14. A compound of formula (I): 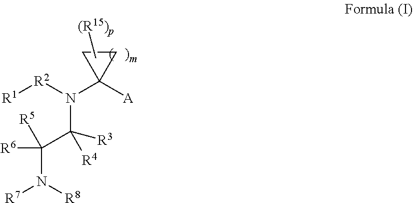
wherein
R¹ is —OC₁₋₈ alkyl, —C₁₋₈ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R³ is H, a bond, —OH, or C₁₋₈ alkyl, wherein one or more methylene group optionally and individually is replaced by —O— and/or substituted with ═O;
R¹⁵ is individually selected from the group consisting of C₁₋₃ alkyl, —OH, —CN, and —F;
anyone of R³, R⁴, R⁵, R⁶, R⁷, and R³ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
X is halogen;
m is an integer of 1 to 4; and
p is an integer of 0 to 10;
or a pharmaceutically acceptable salt thereof,
with the proviso that p is not 0 when m is 1,
or the compound according to any one of claims 1 to 14 for use in medicine.

15. A compound of formula (I): 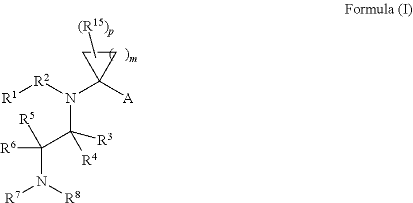
wherein
R¹ is —OC₁₋₈ alkyl, —C₁₋₃ alkyl, optionally substituted with —OH, or H;
R² is a bond, —C(O)—, —S(O)₂—, or —C(H)₂—;
R³ is H, C₁₋₅ alkyl, or a bond;
R⁴ is H, C₁₋₅ alkyl, or a bond;
R⁵ is H, a bond, or C₁₋₈ alkyl, wherein one methylene group optionally is replaced by —O—;
R⁶ is H, a bond, or C₁₋₈ alkyl, wherein one methylene group optionally is replaced by —O—;
R⁷ is H, a bond, —OH, or C₁₋₈ alkyl, wherein one or more methylene group optionally and individually is replaced by —O— and/or substituted with ═O;
R³ is H, a bond, —OH, or C₁₋₈ alkyl, wherein one or more methylene group optionally and individually is replaced by —O— and/or substituted with ═O;
R¹⁵ is individually selected from the group consisting of C₁₋₃ alkyl, —OH, —CN, and —F;
anyone of R³, R⁴, R⁵, R⁶, R⁷, and R⁸ optionally is linked together to form a ring;
A is a phenyl or a pyridinyl, wherein the phenyl or pyridinyl is optionally substituted with one or more substituents R¹³ individually selected from the group consisting of halogen, —CX₃, —OCX₃, —CHX₂, —OCHX₂, —CH₂X, —OCH₂X, —CH₂CX₃, OCH₂CX₃, —C₁₋₈ alkyl, —OC₁₋₈ alkyl, —C₃₋₇ cycloalkyl, —OC₃₋₇ cycloalkyl, —CN, NO₂, —SO₂CH₃, and —SF₅;
X is halogen;
m is an integer of 1 to 4; and
p is an integer of 0 to 10;
or a pharmaceutically acceptable salt thereof,
with the proviso that p is not 0 when m is 1,
or the compound according to any one of claims 1 to 14 for use in the treatment of inflammatory bowel disease (IBD), such as ulcerative colitis or Crohn's disease, hereditary xerocytosis and/or acute respiratory distress syndrome (ARDS).
